(19) United States
(12) Patent Application Publication
(10) Pub. No.: US 2023/0227830 A1
(43) Pub. Date: Jul. 20, 2023

(54) METHODS AND COMPOSITIONS OF RNA NANOSTRUCTURES FOR REPLICATION AND SUB-GENOMIC EXPRESSION BY RNA-DIRECTED RNA POLYMERASE

(71) Applicant: HALO-BIO RNAI THERAPEUTICS, INC., Seattle, WA (US)

(72) Inventor: Todd HAUSER, Seattle, WA (US)

(21) Appl. No.: 18/002,216

(22) PCT Filed: Jun. 18, 2021
(86) PCT No.: PCT/US2021/038083

§ 371 Date: Dec. 16, 2022

Related U.S. Application Data
(60) Provisional application No. 63/041,890, filed on Jun. 20, 2020.

(51) Int. Cl. C12N 15/113; C07K 14/005; A01N 63/60; A01N 25/26; C12N 15/115

(52) U.S. Cl. CPC C12N 15/1131; C07K 14/005; A01N 63/60; A01N 25/26; C12N 15/115; C12N 2310/14; C12N 2310/16; C12N 2770/20023

(57) ABSTRACT

The present invention is directed to methods and compositions of RNA nanostructures for replication and/or subgenomic expression of gene modulating single-stranded RNA by RNA-directed RNA polymerase-like proteins and the use of such nanostructures for use in a variety of organisms.

### CROSS REFERENCE TO RELATED APPLICATIONS

This application claims priority to U.S. Provisional Application No. 63/041,890, filed Jun. 20, 2020, all of which is incorporated herein by reference in its entirety, including drawings.

### INCORPORATION BY REFERENCE OF MATERIAL IN ASCII TEXT FILE

This application incorporates by reference the Sequence Listing contained in the following file:

a) File name: 0276-003WO1_20210618_Sequence_Listing.txt; created Jun. 18, 2021, 345 KB in size.

### BACKGROUND OF THE INVENTION

### Field of the Invention

The present invention relates generally to compositions and methods for RNA-directed transcription derived from highly-structured single-stranded RNA nanostructures. More specifically, the invention presents methods and compositions of RNA nanostructures for RNA-directed RNA polymerase (RdRp) driven replication of the nanostructure and/or the sub-genomic expression of specific sequences of the RNA nanostructure template when in the presence of proteins with RdRp-like properties. The RNA-directed transcription template methods of this invention can selectively elicit a series of subviral events such as nanostructure sequence replication, encapsidation, packaging, and continuous or discontinuous transcription of one or more sequence regions within the nanostructure template leading to potent gene modulation.

### Description of the Related Art

U.S. patent application No. 13/375,460 (Hauser 2010, original MV-siRNA patent), Ser. Nos. 14/954,653, 15/904,224 (Hauser 2016, original polynucleotide nanoparticle patent), and Ser. No. 16/642,244 (Hauser 2015, original aptamer-driven formulation patent) are incorporated herein by reference, including their specifications, which are not admitted to be prior art with respect to the present invention by their mention.

RNA-dependent RNA polymerases (RdRps), also called RNA replicases, is an enzyme that catalyzes the synthesis of RNA from an RNA template and are essential proteins encoded in the genomes of all RNA-containing viruses (Poltronieri et al., 2015). RdRps, present in a wide variety of RNA viruses, are involved in a twostep genome replication, mRNA synthesis, or RNA recombination. They are essential for the survival of viruses and appear to be related to telomerase (Suttle C A et al., 2005). RdRps have multiple conserved sequences and motifs that are essential to the specific recognition of RNA sequences and/or secondary structures of viral genomes that range in length from around a thousand to tens of thousands of nucleotides. For long RNA templates, RdRp's have an error rate (in order of 10⁻⁴) in the transcription process by RdRps leads to genomic variations in the RNA virus population, which is about 1 mutation per 10,000 nucleotides (Domingo and Holland et al., 1997). The switching mechanism during RdRP's copying process may further lead to RNA recombination, and this also helps viruses repair deleterious mutations and acquire new genes and genetic rearrangements.

The most famous example of RdRp is that of the poliovirus. The viral genome is composed of RNA, which enters the cell through receptor-mediated endocytosis. From there, the RNA is able to act as a template for complementary RNA synthesis. The complementary strand is then, itself, able to act as a template for the production of new viral genomes that are further packaged and released from the cell, ready to infect more host cells. The advantage of this replication method is that there is no DNA stage; replication is quick and easy. This general process is shared amongst single-stranded RNA viruses.

Single-stranded RNA viruses are classified as positive or negative, depending on the sense or polarity of the RNA. The positive-sense viral RNA genome can serve as messenger RNA and can be translated into protein in the host cell. Positive-sense ssRNA ((+)ssRNA) viruses belong to Group IV in the Baltimore classification. Positive-sense RNA viruses account for a large fraction of known viruses, including many pathogens such as the hepatitis C, West Nile virus, dengue virus, SARS and MERS coronaviruses, and SARS-CoV-2, as well as less clinically serious pathogens such as the rhinoviruses that cause the common cold. Among other known (+)ssRNA viruses, the Leviviridae are bacteriophages that infect enterobacteria. Yet, the most common ssRNA virus are plant viruses. Members of the (+)ssRNA picornavirus group are so abundant that they are considered dominant.

Eight families of (+)ssRNA viruses infect vertebrates, of which four are unenveloped (Picornaviridae, Astroviridae, Caliciviridae, and Hepeviridae), and four are enveloped (Flaviviridae, Togaviridae, Arteriviridae, and Coronaviridae). All but the arterivirus family contain at least one human pathogen; arteriviruses are known only as animal pathogens (Modrow S et al., 2013). Many pathogenic (+)ssRNA viruses are arthropod-borne viruses transmitted by biting insects, which then transfer the pathogen to animal hosts. Recent metagenomics studies have also identified large numbers of RNA viruses whose host range is specific to insects (Vasilakis N et al., 2015).

All positive-sense ssRNA virus genomes encode RNA-dependent RNA polymerase (RdRp), a viral protein that synthesizes RNA from an RNA template. Host cell proteins recruited by positive-sense ssRNA viruses during replication include RNA-binding proteins, chaperone proteins, and membrane remodeling and lipid synthesis proteins, which collectively exploit the cell's secretory pathway for viral replication.

Coronavirus disease 19 (COVID-19) is caused by a novel coronavirus designated as severe acute respiratory syndrome coronavirus 2 (SARS-CoV-2) (Zhou et al., 2020; Zhu et al., 2020). Like other coronaviruses (order Nidovirales, family Coronaviridae, subfamily Coronavirinae), SARS-CoV-2 is an enveloped virus with a positive-sense, single-stranded RNA genome of ˜30 kb. SARS-CoV-2 belongs to the genus betacoronavirus, together with SARS-CoV and Middle East respiratory syndrome coronavirus (MERS-CoV) (with 80% and 50% homology, respectively) (Kim et al., 2020; Zhou et al., 2020). Coronaviruses (CoVs) were thought to cause enzootic infections in birds and mammals primarily. But, the recurring outbreaks of SARS, MERS, and now COVID-19 have demonstrated the remarkable ability of CoVs to cross species barriers and transmit between humans (Menachery et al.,

Coronaviral RNA contains a “5′-leader” sequence of ˜78 nucleotides (nt) that fuses to the “body sequence,” which is on a downstream part of the genome (Lai and Stohlman et al., 1981; Sola et al., 2015). In the prevailing model, leader-to-body fusion occurs during negative-strand synthesis at motifs called “transcription-regulatory sequences” (TRSs) that are located immediately adjacent to each open reading frame. TRSs contain a conserved 6-7 nt sequence surrounded by variable sequences. During negative-strand synthesis, RdRp pauses when it crosses each TRS in the body (TRS-B), and switches the template to the TRS in the leader (TRS-L), which results in discontinuous transcription leading to the leader-body fusion. From the fused negative-strand intermediates, positive-strand subgenomic RNAs are transcribed.

The viral genome is also used as the template for replication and transcription, which is mediated by nsp12 harboring RNA-dependent RNA polymerase (RdRp) activity (Snijder et al., 2016; Sola et al., 2015: Kim et al., 2020). Negative-sense RNA intermediates are generated to serve as the templates for the synthesis of positive-sense genomic RNA “gRNA” and subgenomic RNAs “sgRNAs.” The gRNA is packaged by the structural proteins to assemble progeny virions. Shorter sgRNAs encode conserved structural proteins (spike protein (S), envelope protein (E), membrane protein (M), and nucleocapsid protein (N)), and several accessory proteins. SARS-CoV-2 is known to have six accessory proteins (3a, 6, 7a, 7b, 8, and 10) according to the current annotation (GenBank: NC 045512.2) (SEQ ID NO:1).

While the invention in this application uses RdRp and RdRp-like proteins for its benefit, RdRp is a popular drug target for small molecule inhibitors by binding to RdRp to antagonize function. Lately, drugs like Sofosbuvir, Ribavirin, Galidesivir, Remdesivir, Favipiravir, Cefuroxime, Tenofovir, and Hydroxychloroquine, have been predictively screened for binding to SARS-CoV-2 RdRp (Elfiky A A. et al., 2020). Some of these RdRp targeting chemicals like Hydroxychloroquine, which was developed as an anti-malaria drug nucleotide analog RdRp inhibitors, which are developed as general antivirals, are candidates for treating viral infections where RdRp's are present (JU J. et al., 2020). Yet, the safety profile and benefits are questionable. For example, Remdesivir is a nucleotide analog, which, despite being promising chemistry, has historically lacked specificity and runs the risk of interacting with other proteins. Hydroxychloroquine has recently been shown in several studies not only to be ineffective in COVID-19 patients but also have raised red flags by being linked to a higher risk of sudden cardiac arrest. In either case, state-of-the-art understanding of viral RdRp's is critical to using them as either an inhibition target (Gao Y., et at., 2020) or as a pro-factor as in this invention.

Self-replicating RNA derived from the genomes of (+) stranded viruses is a powerful tool in developing effective vaccines (TEWS B A et al., 2016). Approaches to developing live attenuated viral vaccines can be exemplified with an example of a Pestiviruse vaccine. Members of the genus Pestivirus are economically important pathogens of farm animals that are grouped in the family Flaviviridae together with their closest relatives, the hepaciviruses. Pestiviruses are the classical swine fever virus (CSFV) and the bovine viral diarrhea virus (BVDV) (Tautz N., et al., 2015). All members of the Flaviviridae are enveloped viruses with positive-strand RNA genomes containing one long open reading frame. The economic impact of pestiviruses results at least in part from causing a wide range of pregnancy disorders and persistent infection due to their ability to cross the placenta in pregnant animals (Tautz N., et al., 2015). Persistently infected animals represent an important reservoir for virus spread. Vaccination represents a feasible means to interrupt the cycle of transmission as long as the vaccines prevent not only disease but also the fetal transmission of the pathogens.

Another approach in vaccine development is based on the deletion of nonessential sequences as described for coronaviruses. Members of the family Coronaviridae represent important pathogens of man and animals, among which SARS and MERS coronavirus (SARS-CoV and MERS-CoV) are best known (Masters P. S., et. al., 2006). Coronaviruses have by far the largest known RNA genomes, which encode not only essential but also some nonessential accessory proteins. Deletion of five of the eight group-specific ORFs (ORF3a, ORF3b, ORF6, ORF7a, and ORF7b), either alone or in various combinations, from the SARS-CoV genomic RNA did not result in clear indications for attenuation in a mouse model. In contrast, a viable SARS-CoV mutant lacking the sequence coding for the E protein (ORF4) was recovered that exhibited reduced virulence in two animal models, probably by the enhanced immune response to the infection (DeDiego M. L., et al., 2007). E represents one of the membrane-bound structural proteins of the virus and is involved in virion assembly and release. Such deletions provide a basis for the development of coronavirus vaccines and are currently the preferred method for live attenuated virus against Coronavirus.

Not only the deletion of sequences but also the exchange of genomic fragments between related viruses is easily done via reverse genetics. It can lead to attenuation and other desired features. As an example, a chimeric pestivirus was established as a vaccine against classical swine fever. This concept was based on replacing the region coding for the central envelope protein E2 of a BVDV genome with the corresponding sequence of CSFV. The resulting virus CP7_E2alf was only able to infect pigs and thus displayed the tropism of CSFV. The chimeric virus was fully attenuated but induced strong protective immunity (Koenig P., et al., 2007). As an important further advantage, the configuration of this chimera allows for serologic differentiation between vaccinated animals and those having been infected by a CSFV field virus, a feature of major importance for control and eradication programs in veterinary medicine (TEWS B A et al., 2016).

The mentioned vaccine methods employ self-replicating RNAs that represent either full-length viral genomes or such RNA with deletion of nonessential sequences that are close to the endogenous composition. Accordingly, these constructs allow the recovery of infectious virus particles. Introduction of the in vitro-transcribed recombinant RNA virus into a cell via transfection starts its autonomous replication leading to the release of infectious viruses that, after amplification in tissue culture, serve as a vaccine. Upon administration, the immune response is triggered because the vaccine virus mimics all steps of a field virus infection without induction of significant symptoms. Such vaccine development methods are useful for prophylactics but have not been applicable to other modes of action or treatment to disease.

The development of improved vaccines to prevent viral diseases in people, domestic and farmed animals remain a considerable challenge. Vaccines are not always effective, and viruses have become resistant to the limited range of antivirals currently available. For example, influenza A virus mutants rapidly became resistant to oseltamivir during the 2009-2010 pandemic (Hurt A. C., et al., 2012). As of June 1st, 2020, the world is currently experiencing a pandemic of SARS-2 nCov (SARS-CoV-2), infecting millions, killing hundreds of thousands, and costing trillions in economic losses. It is, therefore, an urgent need for more effective measures to control the viral outbreaks.

It has been suggested that RNAi, including small interfering RNAs (siRNAs), microRNAs (miRNAs), and synthetic interfering RNAs (sRNAi) may have therapeutic potential (Cullen et al., 2009, Carthew et al., 2009). The siRNAs are made in eukaryotic cells as part of the immune response, while pre-miRNAs are encoded by most eukaryotic cells and some DNA viruses. The siRNA, pre-miRNA, and non-endogenous RNAi triggers like MV-RNA, are enzymatically excised from larger precursors by cellular endonuclease and loaded into an RNA-induced silencing complex (RISC). This RISC complex, with an RNAi guide RNA, binds pre-mRNA or mRNA, and either the complex is cleaved by RISC (MV-RNA, siRNA) or translation is inhibited in some other way (MV-RNA, pre-miRNA). RNAi molecules act similarly when introduced into cells. RNAi has been well known to play important antiviral roles from plants to invertebrates. However, recent studies provided strong supports that RNAi is also involved in the antiviral response in mammalian cells. To combat RNAi-mediated antiviral responses, many viruses encode viral suppressors of RNA silencing (VSR) or produce replication organelles to facilitate their replication and hide from the immune system. A recently published paper identified a novel VSR from coronaviruses, a group of medically important mammalian viruses including Severe Acute Respiratory Syndrome Coronavirus (SARS-CoV) and showed that the Nucleocapsid protein (N protein) of coronaviruses suppresses RNAi triggered by either short hairpin RNAs or small interfering RNAs in mammalian cells (Cui et al., 2015).

Another type of RNA, called Defective interfering (DI) RNAs, differs from other interfering RNAs as they originate from a viral genome and act by competing with viral genomes for replication and/or packaging resources. Their capacity to be packaged provides specific and efficient targeting of DI RNAs, which cannot yet be achieved for the other inhibitory RNA approaches, although progress is being made (Schmid S et al., 2014).

Defective Interfering (DI) genomes are made by nearly all viruses and are antiviral by definition. In the years following their initial characterization, DI viruses were considered to have exciting clinical potential, but translating in vitro promise into in vivo activity proved problematic (Barret A. D. T., et al., 1986). After decades of slow progress, the application of new techniques is moving the field rapidly forward. In particular, cloning technology has been applied to the influenza virus to generate a homogeneous population of DI genomes that has afforded a previously lacking consistency and reproducibility in experimentation. Defective Interfering genomes and particles are now promising “prodrugs” and also “provaccines” that require only minuscule amounts to be clinically effective due to a 10,000× amplification, immediate activity, a systemic spread throughout infected cells, protection against infection, and a natural decay (Dimmock et al., 2012). However, DI genomes have limited modes of actions other than those utilized by the endogenous genome and rely primarily upon immune recognition and as a resource competitor with the target helper virus as a means to reduce viral infections.

Despite years of developments for vaccines, DI genomes and particles, antibodies, and small molecule drugs, there remains a need for methods and compositions to develop novel biological treatments for viral infections. While RNAi appears promising, the endogenously produced siRNA have been derived from dsRNA intermediary templates that are produced in low molarity by viruses, are antagonized by viral RNA suppressors that utilizing the dsRNA binding domains, and require far too much material to be clinically effective due to a lack of replication and packaging. On the other hand, Di genomes require far less material than RNAi treatments and can easily spread cell to cell by utilizing endogenous packaging signals but offer only resource competition and general immune recognition as its mode-of-action.

### BRIEF SUMMARY OF THE INVENTION

This invention provides novel methods and compositions of single-stranded RNA nanostructures that contain a central sequence region of tandem repeat secondary structures, optional subgenomic transcription promoting sequences, and/or a 5′ flanking leader sequence, and/or a 3′ flanking terminal sequence for the replication of all or part of the RNA nanostructure template RNA (FIG. 1). Such RNA nanostructures are engineered for continuous or discontinuous replication and/or act as an RNA vector for the subgenomic expression of specific ssRNA transcripts flanked or containing a subgenomic promoter-like Transcription Regulatory Sequence (TRS) (FIG. 1-3, 7) or intergenic regions, when in the presence of specific RdRp or RdRp-like proteins (FIG. 9iii, 10i-iii) that recognize the associated sequences flanking the central sequence or TRS embedded within. This invention further illustrates the use of RNAi inducing single-stranded secondary structures appended with protein-binding aptamers targeting the structural protein(s) of a host virus as a means to artificially embed the RNA nanostructure template into the lifecycle of the endogenous viral infection within a host (FIG. 1, 3, 4, 6).

This invention provides novel methods and compositions for the synthesis of reverse-complement (−) ssRNA and formation of a dsRNA intermediate (FIG. 2B) from the (+) oriented RNA nanostructure template, and/or expression of additional (+) oriented ssRNA transcripts (FIG. 2A) in a molar excess to the dsRNA intermediate—either of which is derived by specific sequence motif(s) located internally on the template or full length to the RNA nanostructure template.

The RNA nanostructure templates of this invention and/or subgenomic single-stranded RNA transcripts can contain biologically active single-stranded RNA sequences. Such activity can be amplified by this invention due to the overproduction of single-stranded RNA over that of the dsRNA intermediate and original single-stranded RNA template. Without limitation, such over-expressed subgenomic templates or transcripts can be one or more MV-RNA, shRNA, pre-miRNA, sRNA, tRNA, aptamers, or other non-coding RNAs, as well as additional coding RNAs (FIG. 8iii, FIG. 10i).

In preferred embodiments, the novel RNA nanostructures of this invention are flanked by either a minimal polymerase specific 5′ leader, with or without a cap structure, and/or a minimal polymerase 3′ UTR terminal sequences of a modeled RNA virus (FIG. 7, 8, 11) and at least lead to the synthesis of (−) oriented RNA from the nanostructure template sequences forming dsRNA with the RNA nanostructure template which, when combined with a TRS or intergenic signal, can promote subviral transcription of partial or full copies of the (+) RNA nanostructure template sequence. Neither are full genomes of a modeled RNA virus are produced in a molar excess over the RNA nanostructure template.

In certain preferred embodiments, the novel RNA nanostructures of this invention are flanked by only a 5′ leader, with or without a cap structure, or a 3′ UTR terminal sequences of a modeled RNA virus capable of triggering only the (−) reverse-complement and not a new (+) positively-oriented copies of the RNA nanostructure template when in the presence of RdRp or RdRp-like proteins. In such cases and without limitation, such replication-deficient 5′ leader or 3′ UTR sequences may be derived from mutated sequence or deleted elements in the 5′ leader or 3′ UTR sequences, but still contain subgenomic promoter elements to elicit subgenomic expression of part of the RNA nanostructure template. In other cases, the 5′ leader or 3′ UTR sequence is omitted from the nanostructure sequence to create replication-deficient RNA nanostructure templates capable solely of subgenomic expression.

In other embodiments, the 5′ leader and/or 3′ UTR sequences may contain coding sequences for the expression of a specific protein(s).

In yet other embodiments, this invention's novel nanostructure methods and compositions do not contain coding sequences for viral RdRp protein(s). In such cases, only cells infected by the model virus (i.e., coronavirus) would result in RdRp transcription from the RNA nanostructure template (FIG. 7, 8, 9Biii, 10i, 10ii).

In yet other embodiments, the novel RNA nanostructure templates of this invention are capable of promoting RdRp subgenomic transcription of one or more specific ssRNA transcripts sequence(s) templated by a promoter-containing internal sequence (TRS) within the RNA nanostructure sequence (FIG. 8iv). Such internal sequence motifs can be oriented in the (+) or (−) orientation relative to its functional orientation and chosen depending upon amplification preferences. In either case, sequences are proceeded by, or contain a promoter sequence and/or a polymerase specific stem-loop secondary structure, or both (FIG. 3).

This invention provides novel methods and compositions for transcription amplification of (−) oriented transcript compared to the RNA nanostructure template.

This invention provides novel methods and compositions for transcription amplification of (+) oriented transcript compared to the RNA nanostructure template.

In preferred embodiments, the novel RNA nanostructures of this invention are capable of promoting the transcription of non-coding RNA subgenomic transcripts continuously when in the presence of specific RdRp or RdRp-like proteins (FIG. 8iii, 9i, 9ii).

This invention ideally incorporates (+) oriented MV-RNA sequences within the RNA nanostructure sequence, which leads to amplification of (+) oriented MVRNA sequences, which are gene modulating (FIG. 3Ai, 3Aii, 10iii). Additionally, this invention can be composed of (−) oriented MV-RNA or other RNAi triggers along with (+) oriented aptamer sequence regions within the RNA nanostructure sequence, which leads to the subgenomic transcription/amplification of (+) oriented gene-modulating transcripts and (−) oriented aptamer regions—but a nanostructure wrapped in aptamer-bound proteins (FIG. 3Biii, 3Biv). Such transcription events can be triggered by one or more RNA structures and/or transcription-regulatory sequences (TRS) at the end of the 5′ leader sequence and embedded within the RNA nanostructure sequence.

In certain embodiments, the aptamer(s) within the nanostructure binds to viral proteins associated with packaging, and the nanostructure sequence of this invention lacks an endogenous packaging signal (PS). For example, Coronavirus contains a PS sequence near the 3′ end of the ORF1b of ˜70 nt sequence motif within the y7 required for Nucleocapsid protein binding and packaging into the final virion (Hsieh P. K., 2005).

Additionally, this invention contains non-endogenous TRS sequences (FIG. 8iv eTRS, SEQ ID NO:5) instead of endogenous TRS sequences (SEQ ID NO:4). The use of engineered TRS sequences is a method used in the design of live attenuated vaccines to prevent recombination. Even though this invention is not specifically intended to make a live attenuated vaccine that expresses the majority of the viral genome, the use of non-endogenous TRS sequences is preferred embodiments to prevent recombination of this invention with endogenous virus genomes due to the simultaneous presence of this invention along with endogenous virus genomes within the same cell.

Ideally, this invention is dependent upon viral RdRp-like proteins present only within infected cells to function. Other cells, like cancerous cells, are known to have over-expression of TERT protein, which can have an RdRp-like function with this invention (FIG. 14). Alternatively, RdRp related proteins can be co-expressed in the RNA nanostructure template or as a “sgRNA” (FIG. 1-2, FIG. 11B, FIG. 12Ai) of this invention or present as aptamer-bound endoproteins (FIG. 4Ai, FIG. 4Bv).

The novel RNA nanostructure templates of this invention are capable of producing 3′-to-5′ reverse complement transcripts of the 5′-to-3′ RNA nanostructure sequence resulting in the conversion of the secondary structure from that of a highly structured single-stranded form into that of a linear double-stranded RNA (FIG. 14). Upon transcription by an RdRp-like protein(s), such as TERT, a helix is formed due to the binding of the transcript to the template sequence. This dsRNA can result in further transcription, translation, immune stimulation, or direct processing by RNA processing proteins (FIG. 14Cv-viii).

In yet other embodiments, the required RdRp or RdRp-like proteins are either attached to the RNA nanostructure by RNA aptamer sequences (FIG. 4Aiii, FIG. 4Bv “Endoprotein”) or contained as an mRNA within the polynucleotide sequence of the RNA nanostructure template, or co-packaged in the nanoparticle as a mRNA sequence.

In further embodiments, such proteins are endogenous proteins that bind to the aptamers on the RdRp-produced transcript and lead to additional modalities such as intracellular packaging or enveloping (FIG. 6, #3)

In further embodiments, other proteins are either attached to the RNA nanostructure by RNA aptamer sequences (FIG. 4Bv “Endoprotein”) or contained as coding mRNA sequencing within the polynucleotide sequence of the RNA nanostructure template to co-express proteins (FIG. 11Aii, FIG. 12Ai).

### BRIEF DESCRIPTION OF THE DRAWINGS

FIG. 1: RdRp-driven RNAi trigger expressing nanostructure template. FIG. 1 shows an exploded view of an example RNA nanostructure template RNA structure with internal TRS sites in between every 3rd MV-RNA RNAi trigger to denote subgenomic transcript regions, and aptamers on one arm of each MV-RNA.

FIG. 1i: shows a leader region that is mostly a viral 5′ UTR sequence derived from the genome of the RNA virus expressing the RdRp being recruited by this invention or a leader region that has a matching secondary structure to the originating viral genome 5′ region despite being of a different sequence composition (i.e., FIG. 8viii-ix).

FIG. 1ii: shows an engineered 5′ TRS different from the virus being mimicked to avoid recombination.

FIG. 1iii: shows transcript 1 of the 8 total transcripts of the example template.

FIG. 1iv: shows a few of the aptamers contained in this RNA nanostructure template example. Such aptamers can be the same or different. When nanostructure replication and packaging are preferred, at least a few aptamers will be designed to bind to the encapsidation or envelope proteins of the endogenous virus being mimicked.

FIG. 1v: shows one of the several engineered internal TRS motifs that are different from the virus being mimicked to avoid recombination.

FIG. 1vi: shows a 3 ‘UTR region that is mostly a viral 3’ UTR sequence derived from the genome of the RNA virus expressing the RdRp being recruited by this invention or a 3′ UTR region that has matching secondary structures to the originating viral genome 3′UTR despite being of different sequence composition (i.e., FIG. 8I).

FIG. 2: Nanostructure template for RdRp replication and transcription. FIG. 2 shows an RNA nanostructure motif layout with a 5 ′leader, 3 ′UTR, and TRS sequence motifs in between internal transcript templates within the whole RNA nanostructure template.

FIG. 2A: shows the (−) oriented transcript as the result of strand-switching at the TRS site during RdRp replication of the RNA nanostructure template.

FIG. 2B: shows the (+) oriented transcript as the result of strand-switching at the TRS using (−) intermediary transcripts bound to the nanostructure TRS as the RdRp template.

FIG. 3: Sequence orientation for (+) or (−) sub-genomic transcripts. FIG. 3 compares two methods of orienting a transcript fragment from an RNA nanostructure template where either (−) or (+) transcripts are active RNAi triggers and whether the (−) or (+) transcript has an aptamer in the correct orientation for binding a protein.

FIG. 3Ai: shows (+) oriented nanostructure transcript template where the aptamer is active. On (−) oriented transcript intermediates, the aptamer is inactive due to the reverse-complement orientation of the transcript relationship to the target protein.

FIG. 3Aii: shows (+) oriented nanostructure transcript template where the RNAi is active. On (−) oriented transcript intermediates, RNAi is inactive due to the reverse-complement orientation of the transcripts related to the target gene.

FIG. 3Biii: shows (+) oriented nanostructure transcript template where the aptamer is binding to a target protein. On (−) oriented transcript intermediates, the aptamer is non-binding due to the reverse-complement orientation of the transcript relationship to the target protein. On (+) subgenomic transcripts, the aptamer is binding to a target protein.

FIG. 3Biv: shows (+) oriented nanostructure transcript template where the RNAi is inactive and in reverse-complement orientation within the (+) RNA nanostructure template. On (−) oriented transcript intermediates, RNAi is active due to complementary orientation of the transcripts related to the target gene, and the aptamer is non-binding due to reverse-complement orientation of the aptamer motif which abolishes the binding relationship to a target protein. On (+) subgenomic transcripts, the RNAi is inactive, and the aptamer is binding to the target protein.

FIG. 4: ssRNA core, protein coat, enveloped particle stages. FIG. 4 shows biophysical stages supported by this invention that include a highly structured series of RNAi triggers, a 5′ leader and a 3′ UTR tail as an ssRNA core, an aptamer-driven protein encapsidated, and packaging into a final membrane enveloped particle.

FIG. 4Ai: shows the highly-structured ssRNA core.

FIG. 4Aii: shows aptamer sequences presenting on the surfaces of the ssRNA core.

FIG. 4Aiii: shows an endoproteins bound to one or more aptamers on the ssRNA core.

FIG. 4Aiv: shows the endoprotein encapsidated ssRNA core enveloped by lipid, membrane or ectoproteins.

FIG. 4Av: shows an enveloped nanoparticle with an ssRNA core, aptamer-bound endoprotein coat, and an encasing lipid containing a viral membrane protein, envelope protein, and ectoprotein (spike) protein.

FIG. 5: Coronavirus life cycle. FIG. 5 shows the five stages of an example coronavirus lifecycle which includes; endocytosis, translation of non-structural proteins, replication of the viral genome, subgenomic expression from the viral genome, packaging of new viral genomes, and export of new coronavirus virions.

FIG. 6: Example life cycle of this invention. FIG. 6 shows the four stages of an example of this invention in a cell infected with Coronavirus which includes; 1. Entry (endocytosis), 2a. replication of the RNA nanostructure template by hijacking infection-associated non-structural proteins, 2b. subgenomic expression from the RNA nanostructure template by hijacking infection-associated RdRp's, 3. packaging of new RNA nanostructure templates by forming a coating composed of infection-associated Nucleocapsid, and 4. export of new nanostructure virions by hijacking infection-associated Membrane, Envelope, Nucleocapsid, and Spike proteins that were expressed due to infection by Coronavirus. In 2b, transcription of subgenomic transcripts occurs. Expressed transcripts can be RNAi triggers, non-coding, or coding RNA.

FIG. 7: Model of SARS-2 Coronavirus genome conversion. FIG. 7 shows a map view of the Coronavirus genome and locations of the extracted motifs for 5 ′ leader, 3 ′UTR, and an engineered TRS (eTRS) to create a transcription regulatory network template in which RNAi sequences can be inserted into as subgenomic transcripts (sgRNA) when in the presence of coronavirus infected cells.

FIG. 7i: shows a map view of the 5′ leader region derived from the SARS-2 Coronavirus 5′ UTR and continuing through a portion of the SARS-2 ORF1 region as the 5′ leader of this invention example. Such 5′ leader regions can be modified by changing the leader TRS sequence or intergenic regions found in RNA virus genomes with subviral activity. This example is drawn from the 5′ UTR of the SARS-2 Coronavirus (SEQ ID NO: 1) and modified as (FIG. 8B, SEQ ID NO: 2, SEQ ID NO: 5-6, SEQ ID NO: 20) or appended with mRNA sequences at the 3′ end (FIG. 8iii).

FIG. 7ii: shows a map view of the body TRS sequences that act as subviral promoters for flanking sequences for SARS-2 (SEQ ID NO: 4). Such body TRS sequences may be modified (SEQ ID NO: 5) in tandem with the leader TRS motif to create an engineered TRS “eTRS” subviral transcription network within the RNA nanostructure template and such templates perform as a non-endogenous RNA vector.

FIG. 7iii: shows a map view of the subviral or subgenomic transcript regions “sgTranscripts” that are expressed (FIG. 8D) from and in excess to the RNA nanostructure template overall.

FIG. 7iv: shows a map view of the 3′ UTR derived from the SARS-2 Coronavirus genome as the 3′ UTR of this invention example.

FIG. 8: Example of SARS-2 conversion to suppress SARS-2 virus. FIG. 8 shows a map view of an example (SEQ ID NO: 20) of this invention derived from SARS-2 Coronavirus in which the invention is designed to selectively suppress SARS-2 infections when introduced to at least some SARS-2 infected cells of a host. Without limitation, the flanking regions are derived from an engineered 5′ UTR leader (i) and the endogenous 3′ UTR (v) of the SARS-2 genome (SEQ ID NO: 1), contains an mCherry protein CDS (iii) (SEQ ID NO: 20, nucleotides 303 . . . 1013), an optional RNA features (iv) region composed of a SARS-2 Nucleocapsid binding aptamer (SEQ ID NO: 20, nucleotides 1030 . . . 1117) and a predicted SARS-2 packaging signal (SEQ ID NO: 20, nucleotides 1120 . . . 1199), two subviral RNAi transcripts that express MV-RNA targeting SARS-2 spike protein (iv). The endogenous TRS network was modified with an eTRS motif in both the leader region (v) and the subviral promoters (iv). The leader 5′ UTR sequence was further modified to mimic the secondary structure of the native SARS-2 genome despite the sequence differences by modifying sequences (vi, vii).

FIG. 8i: shows a non-limiting map view of an RNA template example SAR-2 treatment (SEQ ID NO: 20) using the design and methods of this invention. The template is made up of an engineered SARS-2 5′ leader sequence, a protein coding region, a region with non-coding RNA, a central region of RNAi single-stranded RNAi triggers with subgenomic (subviral) promoters, and a SARS-2 3′ UTR that includes the proceeding nucleotides of SARS-2 through ORF10.

FIG. 8ii: shows a map view of the 5′ leader sequence that includes an engineered TRS and base modifications (SEQ ID NO: 2) derived from the 5′ end of the SARS-2 Coronavirus genome (SEQ ID NO: 1, nucleotides 1-163).

FIG. 8iii: shows a map view of a coding protein example. In this case, mCherry (SEQ ID NO: 20, nucleotides 303-1013) is the expressed protein and can be used as a transfection and amplification signal (FIG. 10i).

FIG. 8iv: shows a map view of optional RNA features that can be inserted after the coding sequence, but before the subgenomic promoter. Examples include aptamers or RNA packaging signals (SEQ ID NO: 17).

FIG. 8v: shows a map view of a non-limiting example of a central RNA region made up of multiple single-stranded RNAi triggers and two subgenomic promoter sequences to drive subviral expression of the single-stranded RNAi trigger constructs. The sgTranscript I, expresses three MV-RNA (each marked with a SQUARE) that target SARS-2 genes at six different sites and this subviral transcript contains six Mango aptamer fluorescence signals (each marked with a CIRCLE) when in the presence of T01-Biotin, a 150 mM KCl and under 535 nm excitation (FIGS. 9Ai, 9Biii, 10ii). The sgTranscript II, expresses five MV-RNA (each marked with a SQUARE) that target SARS-2 genes at 10 different sites and this subviral transcript contains 6 GFP-like aptamer fluorescence signals (each marked with a CIRCLE) when in the presence of DFHBI, a 150 mM KCl and under 480 nm excitation (FIG. 9Aii, 9Biii).

FIG. 8vi: shows a map view of the 3′ portion of the RNA nanoparticle template that is derived from the 3′ end of the SARS-2 coronavirus (SEQ ID NO: 1, nucleotides 29,558-29,903)

FIG. 8vii: shows a map view of the inclusion of SARS-2 nsp1 (SEQ ID NO: 1, nucleotides 266-301) region included in the 5′ leader construction of this invention in order to replicate the native secondary structure of this example compared to the SARS-2 viral genome (FIG. 8ix vs. FIG. 8x). The start codon “ATG” of nsp1 in this motif was mutated “Ata” to prevent protein coding.

FIG. 8viii: shows a map view of the native TRS (SEQ ID NO: 4) of SARS-2 replaced with eTRS (SEQ ID NO: 5) and the base mutations derived from “GTT” of the SARS-2 genome (SEQ ID NO: 1, nucleotides 61-63) to “GCGA” (SEQ ID NO: 20, nucleotides 61-64) in order to replicate the native secondary structure of this example compared to the SARS-2 viral genome (FIG. 8ix vs. FIG. 8x) after the base changes caused by eTRS.

FIG. 8ix: shows a map view of the 5′ secondary structure of the native SARS-2 genome (SEQ ID NO: 1).

FIG. 8x: shows a map view of the 5 ′leader sequence of this SARS-2 treatment example (SEQ ID NO: 20) matching that of the native virus (FIG. 8ix) despite sequence differences indicated in (FIG. 8vii-viii).

FIG. 9: Example fluorescence in the presence of SARS-2 protein. FIG. 9 shows examples of fluorescence signals derived from the SARS-2 treatment RNA nanostructure template when dyed in a buffer (FIG. 9Ai) and in a human cell lysate (FIG. 9Aii).

FIG. 9Ai: shows the predicted secondary structure, fluorescent aptamer locations, and actual fluorescent signals derived from the subgenomic transcript RNA (central RNA region of the RNA template FIG. 8v) of the SARS-2 treatment (SEQ ID NO:20).

FIG. 9Aii: shows a chart a model of the RNA secondary structure with DFHBI or Mango aptamers in circles and the corresponding fluorescence of aptamer signal in an in vitro reaction. In general, 1 μg of either the treatment example or a negative RNA was added to 50 μl of a 10 mM NaPO4, 150 mM KCl, 1 mM MgCl2 buffer, 200 mM DFHBI or Mango Dye, and incubated for 30 minutes at room temperature. Fluorescent signals were photographed directly on the tube with the use of a Dinoscope USB fluorescent microscope (www.dinolite.com) with either a 535 nm or 480 nm excitation model.

FIG. 9Biii: shows a chart comparing treated vs. untreated, SARS-2 infected vs. uninfected human HEK293 cell lysates 22 hours post-infection. In general, 4 μg of a custom designed non-infectious SARS-2 replicon RNA was added to a high-yield human cell IVT reaction and incubated for 4 hours. Then, 2 μg of the example RNA nanostructure SARS-2 treatment (SEQ ID NO: 20) was added to the reaction and incubated for 18 hours. At the end of the reaction time, 200 mM DFHBI (Green) or T01-Biotin (Orange) was added to blank reactions, treated reactions lacking SARS-2, and treated reactions with SARS-2. The fluorescent signal reflects RNA concentration. No signal was detected in blank reactions. The RNA was detectable in all treated samples, but the RNA was at a higher concentration in the presence of SARS-2, suggesting replication and/or subgenomic expression in the presence of the SARS-2 RdRp complex.

FIG. 10: Example fluorescence, replication and SARS-2 suppression in Vero cells. FIG. 10 shows immunofluorescence of the SARS-2 treatment RNA once transfected into Vero cells that were infected or not infected with Wuhan SARS-2 coronavirus virions at an acceptable MOI and the resulting impact on the production of the Spike protein after treatment. In general, wells of an 8-well plate were seeded with 25000 cells/cm2 and cultured for 24 hours. Some cells were infected with SARS-CoV-2 MOI 3 (24 h incubation) for 1 h.

To test the SARS-2 treatment RNA, 50 μl of a transfection mix (0.25 μl (500 ng) of HaloVIR RNA+1 μl Lipofectamine 3000 in 50 μl of OptiMEM or transfection without RNA) was added to the wells. After 4 hours, cells were washed with PBS and fixed with 4% formaldehyde 16 or 24 hours post-transfection. For RNA staining, T01-Biotin was used @ 1:200 dilution in low sodium phosphate buffer containing 150 mM KCl. Hoechst was used at 5 μg/ml. 30 min RT to stain nuclei. For Immunostaining, anti-Spike was used. Fluorescence was captured with a Leica SPE confocal 20× and 40× objective excitation/filters standard for mCherry (RED), Mango (shown in GREEN), and GFP (remapped to PINK).

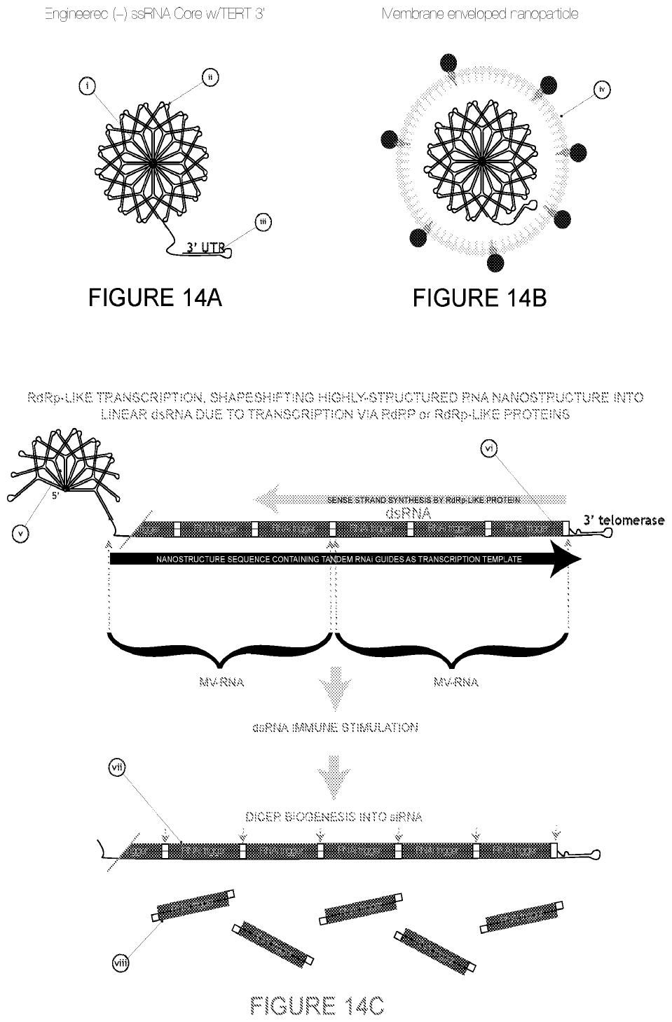

FIG. 10i: shows the mCherry signal (RED) in Vero cells to check for a signal from the protein coding region of the SARS-2 treatment example RNA “HaloVIR” (SEQ ID NO: 20). This row compares mCherry (RED) signals of Vero cells with SARS-2 alone, SARS-2+treatment RNA, or treatment alone. The SARS-2 infected cells that received the treatment RNA indicate an increase in mCherry (RED), indicating that a higher amount of RNA is leading to an increase in mCherry protein expression from the treatment RNA.

FIG. 10ii: shows the Mango signal (remapped to GREEN) in Vero cells to check for a signal from the SARS-2 treatment example RNA “HaloVIR” (SEQ ID NO: 20). The results indicate that the example treatment RNA is replicated by the SARS-2 infection. There is high background, but a clear increase in RNA content in SARS-2 infected cells vs. non-infected treated cells.

FIG. 10iii: shows the SARS-2 spike protein resulting from infection (remapped to PINK) in Vero cells. Cells treated with SARS-2 treatment example RNA “HaloVIR” (SEQ ID NO: 20) appear to indicate a much lower level of SARS-2 spike protein production 16 hours after treatment.

FIG. 11: Example modeled from ssRNA Dicistrovirus to target host. FIG. 11 shows a map view of an insect Dicistrovirus, DVVv3 (SEQ ID NO: 35); the 5′ UTR (SEQ ID NO: 36), ORF1 (SEQ ID NO: 35, nucleotides 676-9,193), intergenic region (SEQ ID NO: 38), ORF2, ORF2 structural protein (SEQ ID NO: 35, nucleotides 9235-12,456), 3′ UTR (SEQ ID NO: 37) and how to apply the designs and methods of this invention to this viral model.

FIG. 11Ai: shows the gene view of this example virus, DVVv3 (SEQ ID NO: 35).

FIG. 11Aii: shows the viral genome of DVVv3 ORF2 (nucleotides 9235-12,456) replaced with an MCS and fluorogenic aptaers (FIG. 11D, SEQ ID NO: 40) for a GFP-like signal from the RNA and a non-infectious replicon.

FIG. 11Aiii: shows a map view the 5′ UTR, intergenic region, the MCS/fluorogenic aptamer region, and the 3′ UTR as a minimum construct for replication and subgenomic expression (SEQ ID NO: 42).

FIG. 11Aiv: shows a map view of an example sequence with a AvrII/KnpI MCS, and five GFP-like fluorescence aptamers.

FIG. 11Av: shows a map view of an example series of single-stranded MVRNA sequences that can be inserted into the constructs above to trigger RNAi-based suppression of four Western Corn Rootworm genes upon the transcription of RNA nanoparticle template. This example RNAi series also contains “Mango” aptamers in-between every 4th MV-RNA for transcript-level tracking of the subgenomic transcription in-vitro or in-vivo.

FIG. 11Biv: shows a graphical view of the secondary structure of the subgenomic RNA that is expressed by (FIG. 11Ai, FIG. 11Aiii), the target genes, and the location of the fluorescence aptamers.

FIG. 12: Template nanostructure replication by specific viral RdRp. FIG. 12 shows the replication of DVVv3 based examples of this invention in either an in-vitro reaction or within a Western Corn Rootworm larva. Designs based on DVVv3 or Providence virus are compared.

FIG. 12Ai: shows the results of an sf21 cell free in-vitro reaction in which the RdRp's of two viruses are potentially expressed by RNA nanostructure templates that either code for the endogenous RdRp or not. Using a T7 reaction and 1 μg of a plasmid with each template; 1. PrV_min (no RdRp), 2. PrV_Rep (RdRp), 3. DVVv3_min (no RdRp), DVVv3_part_rep (RdRp), RNA expression was confirmed over 4 time points by staining with DFHBI. At the 5th timepoint, the RNA nanostructure template for DVVv3 that does not express an RdRp protein was added to all samples and fluorescence was monitored over time.

FIG. 12Aii: shows that only the reactions that expressed DVVv3 RdRp prior were able to maintain a fluorescent RNA signal over time after the addition of the RNA nanostructure template containing DVVv3 UTR's alone.

FIG. 12Biii: shows that larva fed with the RNA nanostructures of this invention example have a specific activity in the target insect and show a fluorescence greater than the control RNA nanostructure template based on a separate non-endogenous virus, Providence Virus.

FIG. 12Biv: shows the DFHBI fluorescence of larva that did not feed on the example RNA nanostructure templates.

FIG. 12Bv: shows the DFHBI fluorescence of larva that fed on control RNA nanostructure templates based on Providence Virus. No signal gain was detected.

FIG. 12Bvi: shows the DFHBI fluorescence of larva that fed on the RNA nanostructure templates based on a minimum DVVv3 construct (FIG. 11Ciii, SEQ ID NO: 42). A GFP-like signal is detected in the gut above the level of the controls suggesting some amplification from endogenous RdRp's from persistent DVVv3 infection of this insect class.

FIG. 12Bvii: shows the DFHBI fluorescence of larva that fed on the RNA nanostructure templates based on a DVVv3_Rep construct (FIG. 11Aii, SEQ ID NO: 39). A higher GFP-like signal is detected in the gut above the level of the controls and DVVv3 min, suggesting a higher amplification when RdRp is co-expressed.

FIG. 13: A model of this invention targeting a host insect.

FIG. 13A: shows a model of 1) the RNA nanostructure templates of this invention loaded into, 2) a capsid by either endogenous or aptamer-driven loading, and 3) the VLP entering the target insect cell, and 4) being amplified by RdRp upon entry—while remaining non-infectious and/or being subviral.

FIG. 13B: The specific example of this invention is based on DVVv3 where the RNA nanostructure template is loaded into the capsid of the DVVv3 virus. The RNA nanostructure is incapable of expressing the DVVv3 capsid, but inherits all of the protective, tropism, and amplification features of this viral machinery upon exposure to the insect and results in a potent amplification of the gene modulating RNA in which it's composed. In some cases, the RNA expressed by this example contains aptamer sequence(s) that systemic transport of the RNA of this nanostructure template.

FIG. 14: Shapeshifting by RdRp-like synthesis of (−) strand. FIG. 14 shows shape-shifting of a highly structured sphere-like nanostructure into a linear dsRNA due to the transcription event caused by the presence of RdRp or an RdRp like protein and a 3 ′UTR with a stable hairpin on the end acting as a primer. In nanostructure form, the conformation for formulation and cellular delivery of a nanostructure is optimal and supports entry alone or by supporting encapsidation before entry. Upon entry into a cell, shape-shifting occurs into dsRNA upon transcription of the complementary strand by RdRp or RdRp-like proteins. TERT is such a protein. The long dsRNA can elicit strong immunological responses in cells and be further processed by dicer-like proteins into siRNA. The sequence of the siRNA is defined by the guide strands of the MV-RNA embedded into the nanostructure of this invention.

FIG. 14Ai: shows the nanostructure of this invention without a 5′ leader or TRS sequence, but a sole 3 ′UTR that contains a stable hairpin to activate TERT-associated RdRp-like activity.

FIG. 14Aii: shows dinucleotide loops on each MV-RNA in support of downstream biogenesis into siRNA with dinucleotide overhangs.

FIG. 14Aiii: shows a stable hairpin on the 3 ′UTR of the ssRNA sequence of the nanostructure.

FIG. 14Biv: shows the nanostructure composition of FIG. VIII A-C loaded into a lipid nanoparticle. The ecto-protein on the lipid membrane is a cell-specific ligand. In most cases, cancer cell binding ligands would be expected.

FIG. 14Cv: shows the nanostructure being unwound and used by RdRp or RdRp-like proteins as a template for strand synthesis in the 3′-to-5′ direction.

FIG. 14Cvi: shows the start of transcription at the 3 ′hairpin RdRp or RdRp-like proteins. A sense strand is synthesized in the 3′-to-5 ′ direction which creates an energetically favorable dsRNA over the prior nanostructure conformation.

FIG. 14Cvii: shows the resulting dsRNA with a new sense strand complementary to ssRNA nanostructure sequence. Each siRNA containing, separated by a dinucleotide is indicated within each MV-RNA converted into siRNA by strand synthesis caused by this invention.

FIG. 14Cviii: shows the resulting siRNA sequences derived from the MVRNA-to-dsRNA shapeshifting, then biogenesis by dicer.

### DETAILED DESCRIPTION OF THE INVENTION

While the invention has been described with reference to the aforementioned specification, the descriptions and illustrations of the preferable embodiments herein are not meant to be construed in a limiting sense. It shall be understood that all aspects of the invention are not limited to the specific depictions, configurations or relative proportions set forth herein which depend upon a variety of conditions and variables. Various modifications of the invention will be apparent to a person skilled in the art upon reference to the present disclosure. It is, therefore, contemplated that the appended claims shall also cover any such modifications, variations, and equivalents.

The practice of various embodiments of the invention employs, unless otherwise indicated, conventional techniques of immunology, biochemistry, chemistry, molecular biology, microbiology, cell biology, genomics, and recombinant DNA, which are within the skill of the art. See Sambrook, Fritsch and Maniatis, MOLECULAR CLONING: A LABORATORY MANUAL, 2nd edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)). As used in the specification and claims, the singular form “a”, “an” and “the” include plural references unless the context clearly dictates otherwise. For example, the term “a cell” includes a plurality of cells, including mixtures thereof.

Technical issues related to fitness and variability make it highly unlikely that effective vaccines can be produced against the majority of them.

Pathogenic RNA viruses are one of the most important groups of pathogens involved in zoonotic transmission events and are a challenge for global disease control. The unknown viral species in the wild and those that have been known for decades—or even centuries—still represent a ongoing problem to human and animal health. Among all potential pathogens that may be involved in interspecies transmissions, RNA viruses are of particular concern. Single-stranded RNA (ssRNA) viruses are major pathogens in every kingdom of life (Schneemann, 2006). RNA viruses are often highlighted as the most common class of pathogens behind new human diseases, with a rate of 2 to 3 novel viruses being discovered each year (Rosenberg, 2015). This class includes infectious agents that are deadly to humans. Studies from the last decades have placed RNA viruses as primary etiological agents of emerging human pathogens, occupying up to 44% of all emerging infectious diseases (ranging from 25% to 44% in different studies), which, along with bacteria (10%-49%), overshadow other parasite groups such as fungi (7%-9%), protozoans (11%-25%), and helminths (3%-6%) (Binder et al. 1999; Jones et al. 2008; Morens et al. 2004; Woolhouse and Gowtage-Sequeria 2005). Their biological diversity and rapid adaptive rates have proven to be difficult to overcome and have stimulated the continuous development of pharmaceutical and medical technology. The technological development specifically designed for the survey and control of RNA viruses must therefore be a research priority. This creates a need for an increased understanding of their fundamental biology to identify novel antiviral therapies.

The replicase proteins, RdRps, are a unique class of nucleic acid polymerase that is both essential and the most conserved protein from RNA viruses. First identified in the 1950s in the mengovirus and poliovirus-related studies (Reich et al., 1961, 1962), the RNA-dependent RNA polymerases (RdRPs) encoded by the RNA viruses catalyze the RNA synthesis from RNA templates, and are responsible for the viral genome replication and transcription processes.

Subviral agents are entities that are simpler than viruses and typically do not encode any proteins, yet rely on a helper virus or prions for infectivity. Such agents lie at the boundary between microbiology and cell biology and utilize RdRp features for replication. Satellite viruses are also subviral agents and depend upon the presence of a helper virus for replication, movement and transmission. Satellite nucleic acids are encapsidated by the helper virus-encoded coat protein, whereas satellite viruses are sometimes able to encode for their own capsid protein and encapsidation. With virus, subvirus, and satellite virus, a host cell is hijacked for the expression and replication of an infectious agent. The methods and compositions of this invention present nanostructures engineered to reclaim “hijacked” cells or use similar mechanisms in the commercial application of subviral particles.

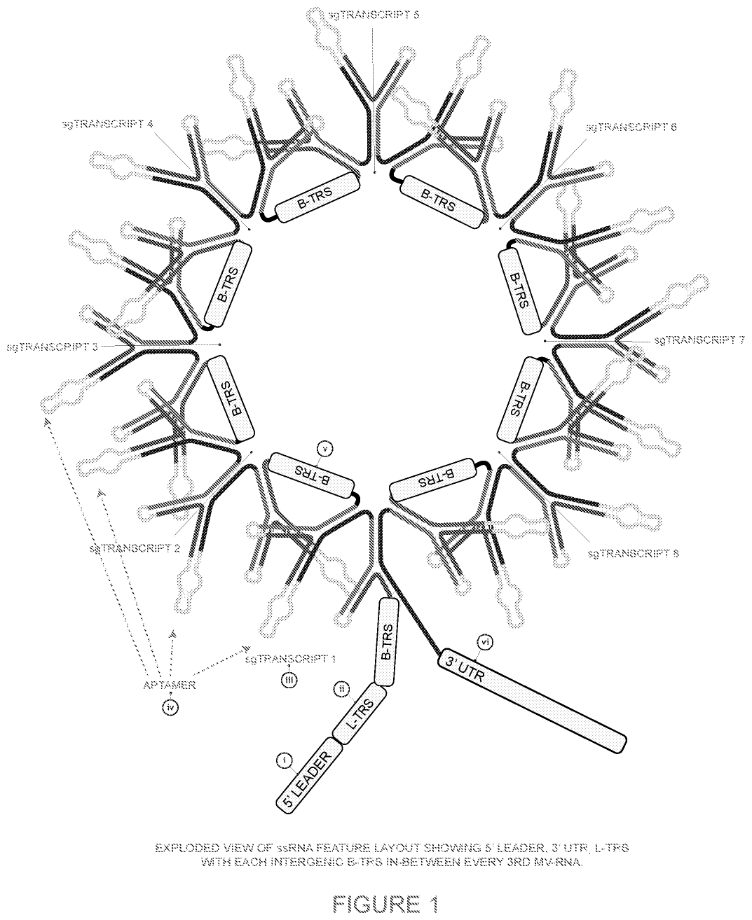

The methods and compositions of this invention introduce novel nanostructures with RdRp features combined with; 1) RNA nanostructures as described in (Hauser, U.S. application Ser. No. 15/904,224) and 2) aptamer-driven protein surfaces also described in (Hauser, U.S. application Ser. No. 16/642,244) to create a novel non-infectious subviral particle platform to induce gene modulation by the over-expression of single-stranded RNA instead of dsRNA traditionally derived from viral methods. This invention is useful for dramatically increase the potency of gene silencing or detection of infections by subgenomic transcription, replication, and/or nanostructure packaging to create subviral particles for use in humans, plant, yeast, bacteria, and animals. This invention further illustrates the use of RNAi inducing single-stranded secondary structures appended with protein-binding aptamers targeting the structural protein(s) of a host-virus as a means to artificially embed the RNA nanostructure template into the lifecycle of the endogenous viral infection within a host (FIG. 1, 3, 4, 6).

This present invention overcomes the limits of siRNA based treatments, defective Interfering RNA, antibodies, and vaccines for the treatment of viral diseases by introducing novel methods and compositions for amplifying the biological activity of non-infectious gene modulating single-stranded RNA with intrans replication, immune stimulation, subgenomic amplification and intracellular packaging of engineered subviral nanoparticles. This invention further enables the development of novel antiviral treatments, therapies for human and animal disease, and broad uses in agriculture. It, therefore, has novel uses in humans, animals, plants, insects, bacteria, and fungus.

Subviral RNA Nanostructure Templates

The RNA-directed transcription template methods of this invention can selectively elicit a series of subviral events such as nanostructure sequence replication, encapsidation, packaging, and continuous or discontinuous transcription of one or more sequence regions within the nanostructure template leading to potent gene modulation.

This invention provides novel methods and compositions of single-stranded RNA nanostructures that contain a central sequence region of tandem repeat secondary structures optionally with embedded subgenomic transcription promoting sequences, and/or a 5 ′flanking leader sequence, and/or a 3 ′flanking terminal sequence for the transcription of all or part of the RNA nanostructure template (FIG. 1).

This invention produces at least one full or partial (−) oriented intermediary reverse-complement transcript of the 5′ to 3′ RNA nanostructure template sequence by including either a 5′ UTR leader sequence, a 3′ UTR, or both.

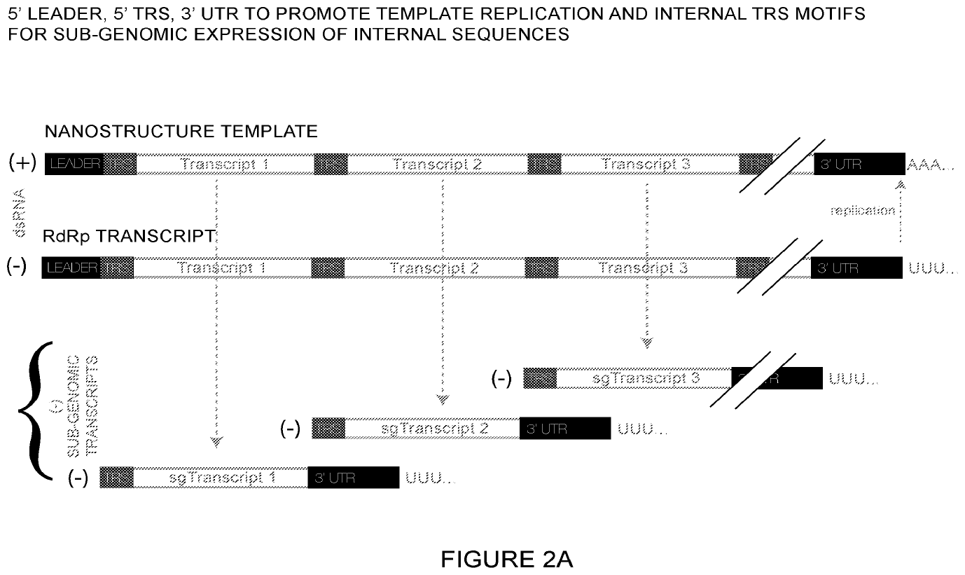

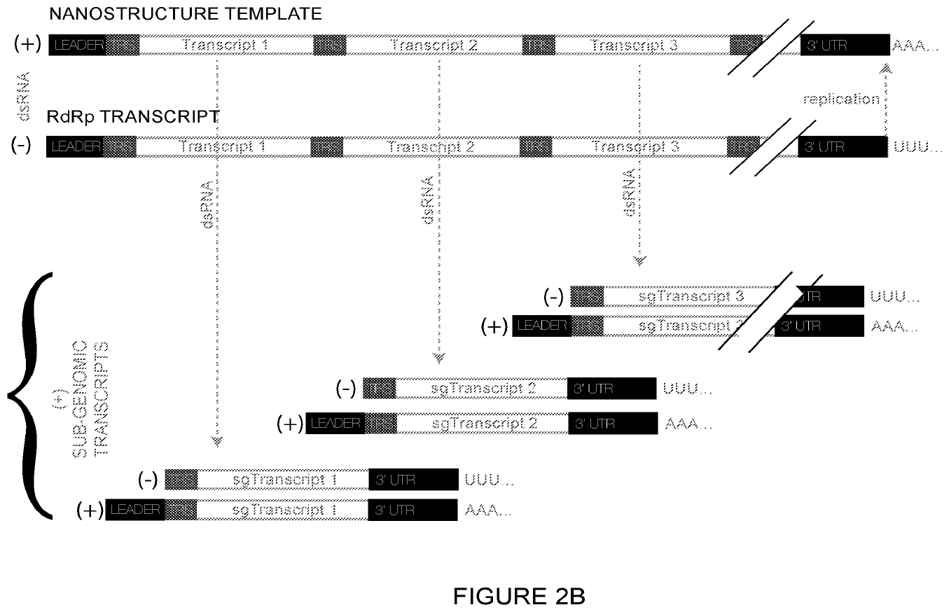

This invention provides novel methods and compositions engineered with a compact secondary structure resulting in biophysical features efficient for cellular uptake, continuous or discontinuous replication and/or act as a template for the subgenomic expression of specific transcripts flanked or containing a Transcription Regulatory Sequence (TRS) or intergenic sequence, when in the presence of RdRp or RdRp-like proteins (FIG. 2).

This invention provides novel methods and compositions for the RNA synthesis of reverse-complement (−) ssRNA transcripts to form a dsRNA intermediate (FIG. 2A), and/or expression of additional (+) oriented ssRNA transcripts (FIG. 2B)—either of which is derived by specific sequence motif(s) internally located on the template. The RNA nanostructure templates of this invention and/or subgenomic RNA transcripts can be biologically active. This invention can amplify such activity due to the molar overexpression of single-stranded RNA over the dsRNA intermediate or the originating single-stranded RNA template. Without limitation, such subgenomic templates or transcripts can be one or more MV-RNA, shRNA, pre-miRNA, sRNA, tRNA, aptamers, coding, or other non-coding single-stranded RNAs.

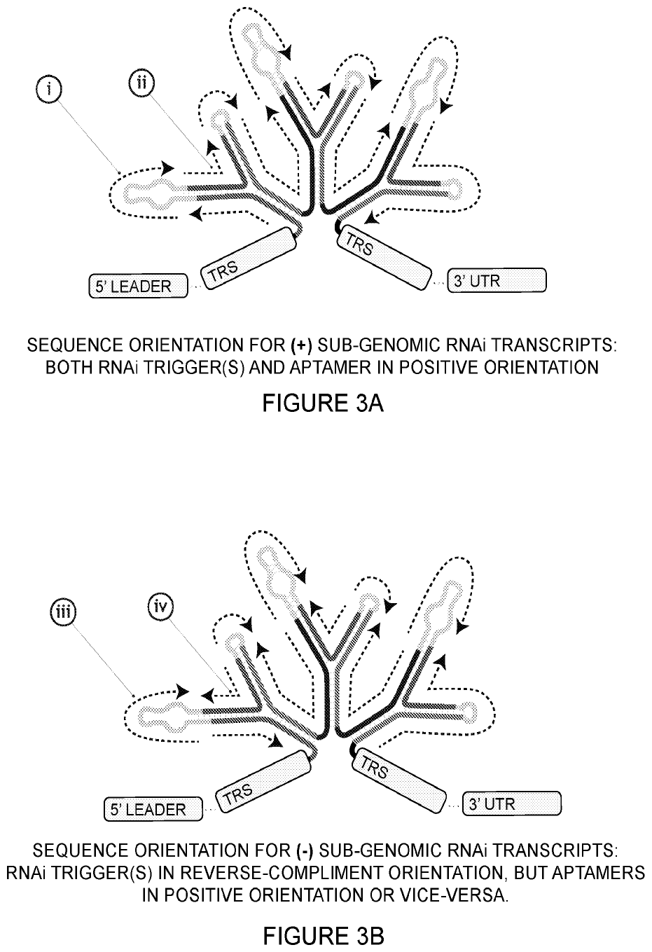

This invention ideally incorporates (+) oriented MV-RNA sequences within the RNA nanostructure sequence, which leads to transcription of (+) oriented and MV-RNA, which are gene modulating (FIG. 3).

Additionally, this invention can be composed of (−) oriented MV-RNA or other RNAi triggers along with (+) oriented aptamer sequence regions within the RNA nanostructure sequence, thus, generating the subgenomic transcription of (+) oriented gene-modulating transcripts and (−) oriented aptamer regions—but a nanostructure wrapped in aptamer-bound proteins (FIG. 3Biii). Such transcription events can be triggered by one or more RNA structures and/or transcription-regulatory sequences (TRS) at the end of the 5 ′leader sequence and also embedded within the RNA nanostructure sequence.

In preferred embodiments, the novel nanostructure methods and compositions of this invention are subviral-lacking coding sequences for protein(s) required as a virus.

In other embodiments, the novel nanostructure methods and compositions of this invention code for protein(s), but do not code for all of the structural proteins of the virus in which the UTR's are derived.

In preferred embodiments, the RNA nanostructure templates of this invention contain uncapped RNA.

In other preferred embodiments, the RNA nanostructure templates of this invention contain a 5′ capped RNA (e.g., type 1 7-methyl guanosine).

In some embodiments, the RNA nanostructure templates of this invention contain a 5′ capped RNA (e.g., type 1 7-methyl guanosine).

In some embodiments, the RNA nanostructure templates of this invention contain a 3′ poly(A).

In other embodiments, the RNA nanostructure templates of this invention lack a 3′ poly(A).

In some embodiments, the RNA nanostructure templates of this invention have a protein covalently bound to the 3′ end.

In other embodiments, the RNA nanostructure templates of this invention have a protein covalently bound to the 5′ end.

In still other embodiments, the RNA nanostructure templates of this invention have one or more protein(s) non-covalently bound to the template.

In some embodiments, the RNA nanostructure templates of this invention have one or more protein(s) non-covalently bound to the template. This protein is an RdRp or RdRp-like protein, or a dicer-like protein, or argonaut protein, or a membrane protein, or other protein(s) which increase the activity of the RNA nanostructure template.

In yet other embodiments, the RNA nanostructure templates of this invention have a second polynucleotide non-covalently bound to the template.

In preferred embodiments, the 5′ and 3′ composition of the RNA nanostructure templates of this invention match the 5′ and 3′ compositions of a modeled RNA virus being modeled in the template design. (e.g., 5′ cap, but no poly(A) when modeling Dengue, or 5′ cap and a poly(A) when modeling SARS coronavirus).

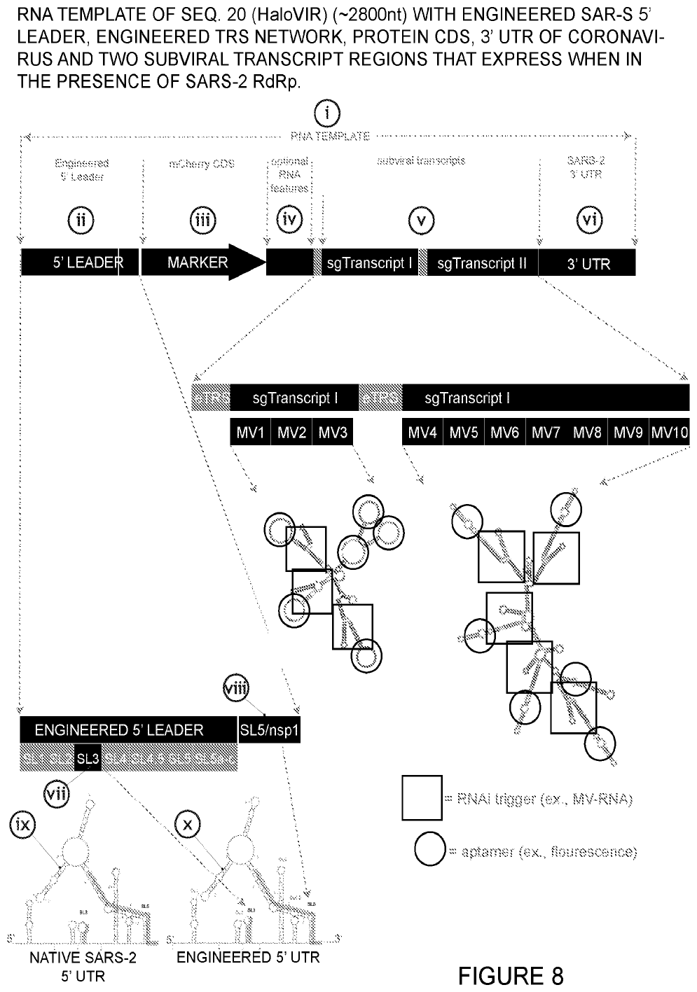

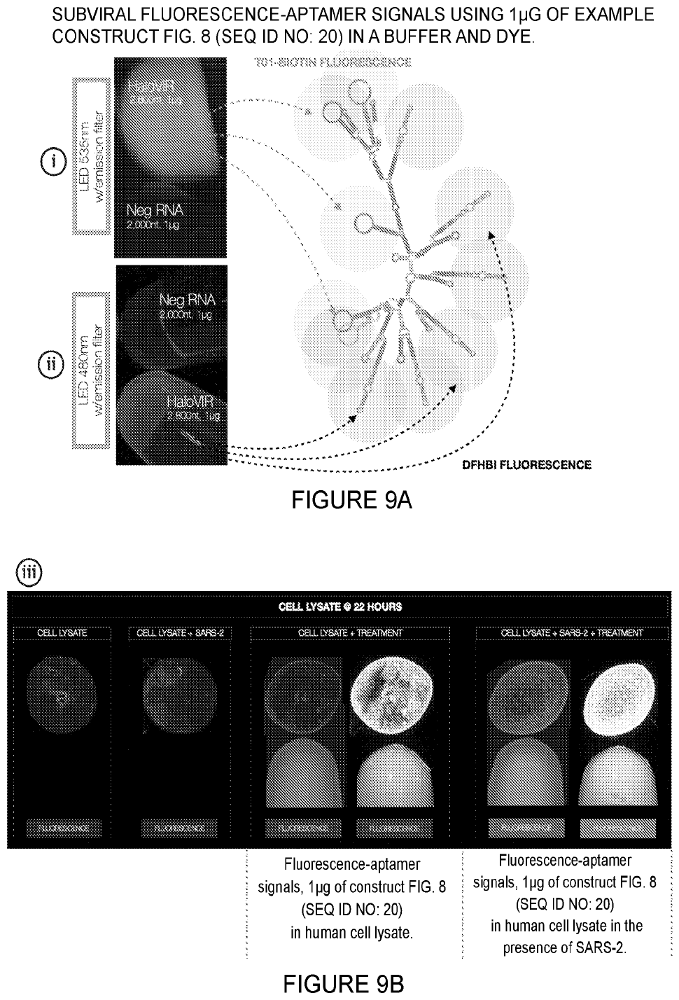

In other preferred embodiments, the 5′ and 3′ composition of the RNA nanostructure templates of this invention have a different sequence but have a matching secondary structure to the 5′ and 3′ compositions of a modeled RNA virus in the template design. (FIG. 8ix).

In some embodiments, the RNA nanostructure templates of this invention have therapeutic purposes of treating a patient suffering from a malady selected from the group consisting of unwanted cell proliferation, arthritis, retinal neovascularization, viral infection, amoebic infection, parasitic infection, fungal infection, unwanted immune response, asthma, lupus, multiple sclerosis, diabetes, acute pain, chronic pain, neurological disease, and a disorder characterized by loss of heterozygosity; comprising the step of:

administering to a patient in need thereof a therapeutically effective amount of a polynucleotide, wherein said polynucleotide is a single-stranded RNA represented by RNA nanostructure template described in this invention, encapsidated by protein as a virion-like particle or enveloped by lipidic proteins as a virion-like particle or formulated with a pharmaceutically acceptable carrier.

In preferred embodiments, the present invention relates to the aforementioned method, wherein said malady is a viral infection (FIG. 1, 7, 8).

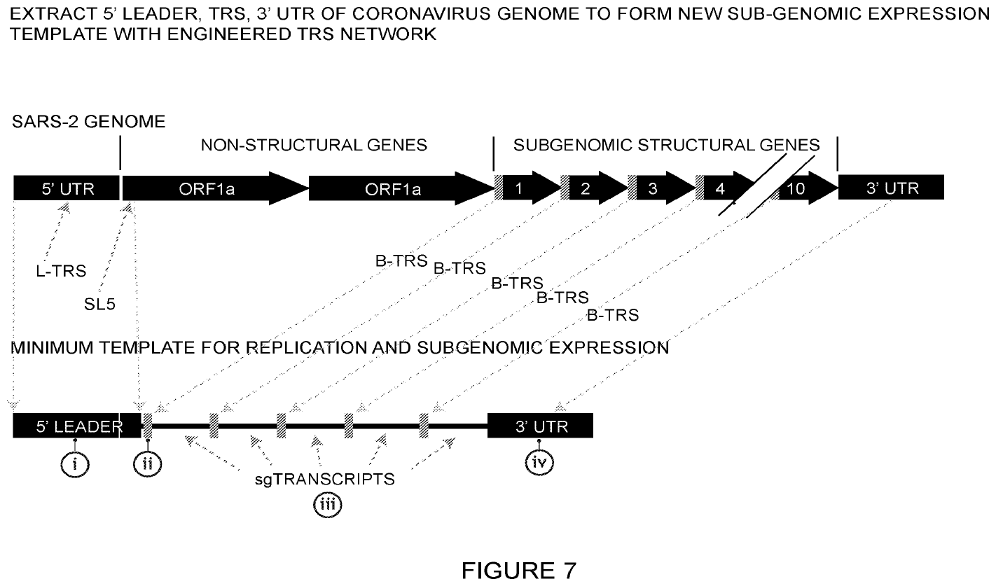

In certain embodiments, the present invention relates to the aforementioned method, wherein said malady is a disorder mediated by Human Immunodeficiency Virus, Hepatitis A Virus, Hepatitis B Virus, Hepatitis C Virus, Hepatitis D Virus, Hepatitis E Virus, Hepatitis F Virus, Hepatitis G Virus, Hepatitis H Virus, Respiratory Syncytial Virus, myxovirt.ls, rhinovirus, coronavirus (FIG. 7, 8) (SEQ ID NO:20), West Nile Virus, St. Louis Encephalitis, Tick-borne encephalitis virus gene, Murray Valley encephalitis virus gene, dengue virus gene, Simian Virus 40, Human T Cell Lymphotropic Virus, a Moloney-Murine Leukemia Virus, encephalomyocarditis virus, measles virus, Vericella zoster virus, adenovirus, yellow fever virus, poliovirus, poxvirus, or other RNA viruses.

In other preferred embodiments, the present invention relates to the aforementioned method, wherein said malady is unwanted cell proliferation (FIG. 14).

In certain embodiments, the present invention relates to the aforementioned method, wherein said malady is testicular cancer, lung cancer, breast cancer, colon cancer, squamous cell carcinoma, pancreatic cancer, leukemia, melanoma, Burkitt's lymphoma, neuroblastoma, ovarian cancer, prostate cancer, skin cancer, non-Hodgkin lymphoma, esophageal cancer, cervical cancer, basal cell carcinoma, adenocarcinoma carcinoma, hepatocellular carcinoma, colorectal adenocarcinoma, liver cancer, male breast carcinoma, adenocarcinomas of the esophagus, adenocarcinomas of the stomach, adenocarcinomas of the colon, adenocarcinomas of the rectum, gall bladder cancer, hamartomas, gliomas, endometrial cancer, acute leukemia, chronic leukemia, childhood acute leukemia, Ewing Sarcoma, Myxoid liposarcoma, brain cancer, or tumors of epithelial origin.

In certain embodiments, the present invention relates to the aforementioned method, wherein said malady is rheumatoid arthritis or retinal neovascularization.

In certain embodiments, the present invention relates to the aforementioned method, wherein said malady is a fungal infection, amoebic infection, parasitic infection.

In preferred embodiments, the intracellularly produced RNA nanostructures of this invention have therapeutic purposes in the treatment of infectious diseases caused by viruses, or fungus, and the treatment is topical, oral, nasal, subcutaneous, respiratory (inhalation), or intravenous for the application of cells containing with said invention.

In still other embodiments, the RNA nanostructure templates of this invention are used for intracellular production during bioprocessing, and the nanostructure template is replicated, encapsidated, optionally packaged, and exported or extracted from a cell or in-vitro reaction to be collected for medical or agricultural uses (FIG. 13).

In some embodiments, the RNA nanostructure templates of this invention are used for crop protection as a biopesticide by targeting the host, but are not limited to these purposes.

Replication

Viruses that possess ssRNA genomes include poliovirus and foot-and-mouth virus (Picornaviridae), Norwalk virus (Caliciviridae), Sindbis virus (Togaviridae), and flavivirus (Flaviviridae). RNA genome replication in ssRNA viruses takes place in a membrane-bound replication complex consisting of the viral polymerase and other viral proteins, cellular proteins, and viral RNA. Viral genomic RNA serves as an mRNA in infected cells to produce viral proteins without any modification of the viral genome, and thus, viral RNA-dependent RNA polymerases are responsible for replication of the viral RNA genome. The RNA-dependent RNA polymerases first transcribe genomic plus-strand RNA into a complementary minus-strand RNA to form a dsRNA intermediate (Uchil and Satchidanandam, 2003). The newly synthesized minus-strand of the dsRNA then serves as a template to generate multiple copies of plus-strand RNA. RNA synthesis occurs in an asymmetric replication cycle in which plus-strand RNA is synthesized in large excess over minus-strand, and all minus-strands exist as a part of the dsRNA intermediate. The replication of a viral RNA genome by RdRp is a fundamental step in the virus life cycle and includes successfully hijacking the host cell machinery for virus production.

Replication defective viruses (also known as satellites) are subviral entities that need the presence of other viruses to help them reproduce. This invention introduces an engineered subviral RNAi nanostructure with replicative dependencies on the presence of RdRp or RdRp-like proteins. This invention's RdRp-dependency is comparable to that of endogenous subviral entities like satellite and replication-defective viruses. However, this invention introduces novel designs and methods for gene modulating nanostructures that can have either subviral modalities when omitting replicase and/or structural proteins in the use, or have viral modalities when including replicase and/or structural proteins in use. In either case, this invention presents novel multifunctional RNA nanostructure scaffolding that combines RNAi triggers, aptamers, and at least an RdRp motif for (−) strand synthesis.

The use of this invention can lead to significant amplification of the RNA activity when expressed when RdRp replicates the RNA nanostructure.

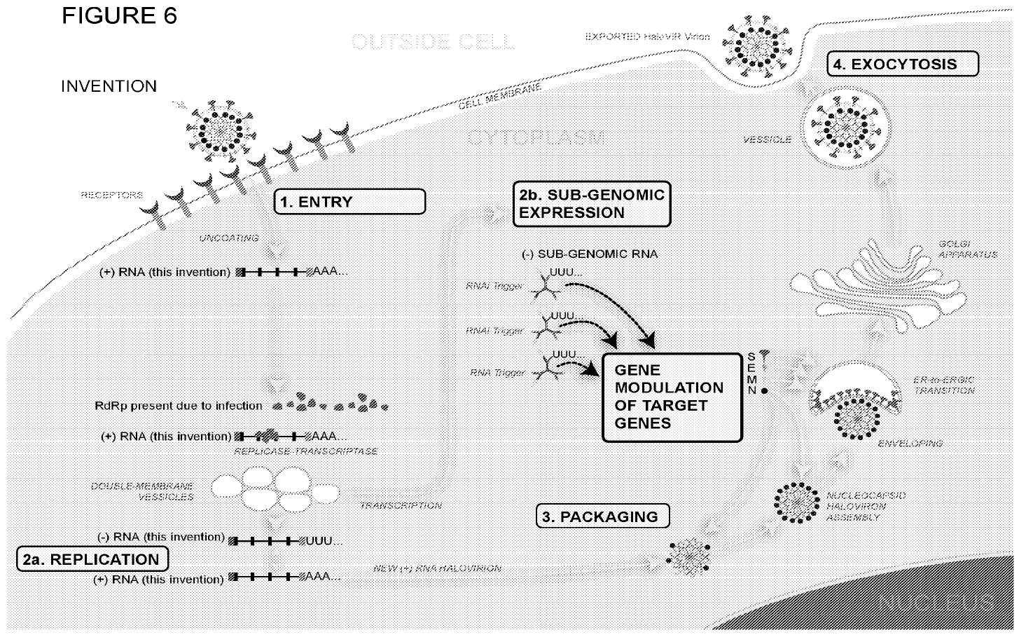

The use of this invention can also lead to significant amplification of the RNA activity when expressed when RdRp replicates the RNA nanostructure, and such replication leads to the packaging and/or exocytosis of new nanoparticle compositions (FIG. 6).

In certain embodiments, the novel RNA nanostructures of this invention are flanked by a polymerase specific 5 ′leader (aka 5′ UTR and additional downstream nucleotides with or without a modified TRS or intergenic region), with or without a cap structure, and/or a 3 ′UTR terminal sequences of a model virus, each typically containing conserve cyclization (CYC) motifs, and lead to the continuous synthesis of additional RNA nanostructure template sequences.

The novel RNA nanostructure templates of this invention are minimally capable of producing 3′-to-5 ′reverse complement transcripts to the 5′-to-3 ′RNA nanostructure template sequence resulting in the conversion of the secondary structure from that of a highly structured single-stranded form into that of a linear double-stranded RNA (FIG. 14). In the case of viral replication, this (−) oriented strand is considered an intermediary sequence.

In preferred embodiments, this invention includes a 5 ′flanking leader sequence and/or a 3 ′flanking terminal sequence derived from a modeled RNA virus with the recruited RdRp protein for the continuous transcription of a (+) oriented transcripts of all or part of the RNA nanostructure template (FIG. 1, 7, 11) and further copies of the (+) oriented RNA nanostructure template.

In other preferred embodiments, this invention includes a 5 ′flanking leader sequence and/or a 3 ′flanking terminal sequence derived from a modeled RNA virus with the recruited RdRp protein for the continuous transcription of a (−) oriented reverse-complement transcript of the RNA nanostructure template that is prematurely terminated and shorter than the full RNA nanostructure template (FIG. 2). In such cases, the RNAi trigger sequences within the RNA nanostructure template are in the reverse-complement orientation within the 5′ to 3′ (+) oriented RNA nanostructure template (FIG. 3Biv).

In other embodiments, the 5′ leader and or 3′ UTR sequences may contain coding sequences for the expression of a specific protein(s).

In certain other embodiments, the 5 ′leader and or 3 ′UTR sequences may contain coding sequences for the expression genes that enhance this invention (e.g., RdRp related proteins, dicer-like proteins, packaging proteins, or RISC proteins).

Subgenomic (Subviral) Transcription

In addition to the use of RdRp for genome replication, numerous RNA viruses generate subgenomic mRNAs (sgRNAs) for the expression of their 3′-proximal genes. These shorter than genome length viral messages allow for the controlled expression of a subset of viral genes. Two different mechanisms for generating sgRNA mRNAs are typical of RNA virus; discontinuous transcription mechanism and associated regulatory schemes used by coronaviruses (Sola et al., 2011), and a premature termination mechanism for subgenomic mRNA transcription (Jiwan and White et al., 2011).

Each RNA virus RdRp has specific promoter elements that may include secondary structure, conserved sequence (CS) motifs (aka, cyclization sequence), intergenic regions (IGR) and even capping RNA-protein interactions to initiate transcription in a primer-dependent or primer-independent manner for sgRNA synthesis by specific promoter elements.

This invention introduces novel designs and compositions to embed promoter elements within the RNA nanostructure template for the subgenomic transcription of a specific portion(s) of the RNA nanostructure template. Ideally, this subgenomic expression is used to express a non-coding, gene modulating single-stranded RNA like MV-RNA, shRNA, pre-miRNA, with or without aptamers for fluorogenic detection, as potent single-stranded gene modulators. The use of this invention can lead to significant amplification of the RNA activity when expressed by subgenomic expression.

In preferred embodiments, some of the sequences of the RNA nanostructure template of this invention are subviral promoter elements derived from a modeled RNA virus and this modeled virus has endogenous subviral promoter elements within it's RNA genome(s) (FIG. 7, 11) (SEQ ID NO: 4, 24, 25, 38).

In such preferred embodiments, a modeled RNA virus of interest has one or more subviral promoter elements such as CS, IGR, secondary structures or protein binding domains sequences (FIG. 7).

In preferred embodiments, the RNA nanostructure template is first derived of a central region containing a plurality of single-stranded gene modulator sequences above and at least one subviral promoter elements from a modeled RNA virus at the 5′ end of the central region (FIG. 11ii, 11iii).

In other preferred embodiments, the RNA nanostructure template is first derived of a central region composed by joining multiple sets of single-stranded gene modulators sequences above, each with a subviral promoter element from a modeled RNA virus at the 5′ end, into a single polynucleotide (FIG. 1, 2).

In some preferred embodiments, the RNA nanostructure has the same number of subviral promoter elements as a modeled RNA virus (FIG. 7, 11), or fewer (FIG. 8) (SEQ ID NO:20).

In yet other embodiments, the maximum number of subviral promoter elements is limited only by the total length of the RNA nanostructure template, including all other sequences, being equal to or less than the sequence length of the genome of a modeled RNA virus.

In certain embodiments, one or more subviral promoter elements have been engineered with a non-endogenous sequences, but maintains the binding interrelationship of the endogenous subviral promoter elements (FIG. 8) (SEQ ID NO: 5, 26, 27), but does not interact with the subviral promoter elements of a modeled RNA virus.

In certain embodiments, the novel RNA nanostructures of this invention are not flanked by both a 5 ′leader (with or without a cap structure) and/or a 3 ′UTR terminal sequences of a model virus capable of triggering both the (−) reverse-complement and a new (+) positively-oriented copy of the RNA nanostructure template when in the presence of RdRp or RdRp-like proteins. In such cases and without limitation, such replication-deficient 5′ leader or 3′ UTR sequences may be derived from mutated sequence or deleted elements in the 5′ leader or 3′ UTR sequences, but contain other sequences with a TRS elements to elicit subgenomic expression of part of the RNA nanostructure template. In other cases, the 5′ leader and/or 3′ UTR sequence is omitted from the nanostructure sequence to create replication-deficient RNA nanostructure templates capable of subgenomic expression.

In yet other embodiments, the novel RNA nanostructure templates of this invention are capable of promoting RdRp subgenomic transcription of one or more specific ssRNA transcripts sequence(s) templated by a promoter-containing internal sequence within the RNA nanostructure sequence (FIG. 2B). Such internal sequence motifs can be oriented in the (+) or (−) orientation relative to its functional orientation and chosen depending upon amplification preferences. In either case, sequences are proceeded by, or contain, a promoter sequence and/or a polymerase specific stem-loop secondary structure, or both (FIG. 2-3).

The RNA nanostructure templates of this invention and/or subgenomic RNA transcripts can be biologically active, and such activity can be amplified by this invention. Without limitation, such subgenomic templates or transcripts can be one or more MV-RNA, shRNA, pre-miRNA, sRNA, tRNA, aptamers, or other non-coding RNAs.

In certain embodiments, the RNAi triggers utilized within this invention target a viral genome and/or the subgenomic transcripts of a virus (e.g., subgenomic mRNA).

In other certain embodiments, the RNA nanostructure and/or subgenomic transcripts of this invention target the virus genome and/or any organism (e.g., plant, animal, protozoan, virus, bacterium, or fungus).

In certain embodiments, the subgenomic transcripts are one or more MVRNA, shRNA, pre-miRNA, sRNA, tRNA, aptamers, or other non-coding RNAs that elicit gene modulation.

In preferred embodiments, the subgenomic transcripts are one or more MVRNA that elicit gene modulation.

In other preferred embodiments, the subgenomic transcripts are one or more MV-RNA that elicit gene modulation. The RNA nanostructure template contains one or more aptamers that bind to structural protein(s) that lead to the packaging of the RNA nanostructure template.

In yet other preferred embodiments, the subgenomic transcripts are one or more MV-RNA that elicit gene modulation and contain one or more aptamers that bind to structural protein(s) that lead to the packaging of the subgenomic transcript.

This invention provides novel methods and compositions for transcription amplification of (−) oriented transcript compared to the RNA nanostructure template and up to 10,000× by using viral RdRp (Dimmock et al., 2012).

This invention provides novel methods and compositions for transcription amplification of (+) oriented transcript compared to the RNA nanostructure template and up to 10,000× by using viral RdRp (Dimmock et al., 2012).

In preferred embodiments, the novel RNA nanostructures of this invention are capable of promoting the transcription of non-coding RNA subgenomic transcripts only when in the presence of RdRp proteins (FIG. 6, 9, 10, 12Ai).

In preferred embodiments, this invention contains engineered non-endogenous subgenomic promoters (e.g., TRS, CS, intergenic, stem-loop, enhancer regions) or other subgenomic motif sequences located in between the 5′ and 3′ UTRs, but flanking either the 5′ or 3′ of the subgenomic transcript within the RNA nanostructure template.

In certain embodiments, a modeled RNA virus is a (+) oriented ssRNA virus, and the subgenomic promoters used in this invention flank the 5′ of the subgenomic transcript within the RNA nanostructure template.

In other embodiments, a modeled RNA virus is a (−) oriented ssRNA virus, and the subgenomic promoters used in this invention flank the 3′ of the subgenomic transcript within the RNA nanostructure template.

In still other embodiments, a modeled RNA virus is a (+) or (−) oriented ssRNA virus, and the subgenomic promoters used in this invention flank the 5′, 3′, or both sides of the subgenomic transcript within the RNA nanostructure template.

In certain embodiments, the RdRp protein, structural proteins and or a modeled RNA viral genome useful in making the templates of this invention are derived from one or more virus families Deltaflexiviridae, Deltaflexivirus, Gammaflexiviridae, Mycoflexivirus, Mitovirus, Tymovirus, Narnavirus, Narnaviridae, Sclerodarnavirus, Victorivirus, Totivirus, Tymoviridae, Zeavirus, Tombusvirus, Totiviridae, Botrexvirus, Chordovirus, Lolavirus, Platypuvirus, Allexivirus, Divavirus, Foveavirus, Mandarivirus, Prunevirus, Robigovirus, Capillovirus, Giardiavirus, Pelarspovirus, Alphacarmovirus, Avenavirus, Citrivirus, Gammacarmovirus, Goravirus, Macanavirus, Machlomovirus, Panicovirus, Potexvirus, Trichovirus, Vitivirus, Alphanecrovirus, Betanecrovirus, Carlavirus, Dianthovirus, Gallantivirus, Leishmaniavirus, Pomovirus, Tobamovirus, Alphaflexiviridae, Aureusvirus, Betacarmovirus, Cystoviridae, Hordeivirus, Pecluvirus, Betaflexiviridae, Tobravirus, Betaendornavirus, Cystovirus, Virgaviridae, Alphaendornavirus, Endornaviridae, Sinaivirus, Tombusviridae, Polemovirus, Bevemovirus, Brambyvirus, Bymovirus, Maculavirus, Blunervirus, Marafivirus, Alphamesonivirus, Ampelovirus, Macluravirus, Mesoniviridae, Roymovirus, Tepovirus, Bafinivirus, Rymovirus, Alphainfluenzavirus, Deltacoronavirus, Kappaarterivirus, Lambdaarterivirus, Okavirus, Roniviridae, Trichomonasvirus, Tritimovirus, Velarivirus, Betanodavirus, Closterovirus, Crinivirus, Deltaarterivirus, Ipomovirus, Poacevirus, Potyvirus, Alphanodavirus, Closteroviridae, Enamovirus, Luteoviridae, Luteovirus, Nodaviridae, Torovirus, Alphaarterivirus, Furovirus, Polerovirus, Potyviridae, Solemoviridae, Torovirinae, Sobemovirus, Alphacoronavirus, Gammacoronavirus, Togaviridae, Alphavirus, Betaarterivirus, Coronavirinae, Flavivirus, Arteriviridae, Betacoronavirus, Coronaviridae.

Recombination

Recombination occurs when at least two viral genomes co-infect the same host cell and exchange genetic segments. RNA viruses are able to undergo two forms of recombination: RNA recombination, which (in principle) can occur in any type of RNA virus, and reassortment, which is restricted to those viruses with segmented genomes. Two RNA recombination mechanisms have been proposed: breakage-rejoining and copy-choice (Nagy et al., 1997).

The breakage-rejoining mechanism takes place with the splicing of group II introns and can result in the production of some recombinant RNAs by the Qβ replicase and is of little interest with this invention. In contrast, copy-choice recombination occurs when the RdRp complex leaves the template (the donor) and resumes synthesis on a second template (the acceptor), and is of significant interest with this invention. The copy-choice mechanism seems to account for the majority of viral RNA recombinants identified thus far (Lai et al., 1992) and this invention significantly reduces and/or eliminates recombination events during it's short replication lifecycle.

This invention utilizes non-endogenous L-TRS and B-TRS sequences and aside from a 5′ or 3′ UTR, shares no homology in the subgenomic regions with a modeled RNA virus (FIG. 7, 8). The use of artificial TRS sequences is a method used in the design of live attenuated vaccines to prevent recombination (Graham et al., 2018). Even though this invention is not specifically intended to make a live attenuated vaccine that expresses viral genes, use of non-endogenous TRS sequences are preferred embodiments to prevent recombination of this invention with endogenous virus genomes due to the presence of this invention along with endogenous virus genomes within the same cell.

In preferred embodiments, an engineered TRS sequence in place of both the Leader-TRS and Body-TRS sequences replaces the endogenous TRS sequences and has no homology to a modeled RNA virus or the host transcriptome.

In still other preferred embodiments, the RNA nanostructure templates of this invention have no homology to the leader-TRS of a host virus.

In still other preferred embodiments, the RNA nanostructure templates of this invention have no homology to the Body-TRS of a host virus.

In preferred embodiments, the RNA nanostructure templates of this invention are recombination deficient with a modeled RNAvirus.

In preferred embodiments, the RNA nanostructure templates of this invention are recombination deficient with RNA of the host.

RdRp-Driven Mutations

Genomic mutation rate, which is the product of the per-nucleotide site mutation rate and the genome size is an important parameter in population genetics. The genomic mutation rate determines the average number of mutations each progeny will have compared to the parental (or ancestral) genome template. On a per-site level, DNA viruses typically have mutation rates on the order of 10⁻⁸ to 10⁻⁶ substitutions per nucleotide site per cell infection (s/n/c). RNA viruses, however, have higher mutation rates that range between 10⁻⁶ and 10⁻⁴ s/n/c, excluding coronavirus, which has some degree of proofreading and a mutation rate range around 9×10−7. Despite variable per-site rates, species with smaller genomes exhibit a negative correlation between genomic mutation rate and genome size, such that the per-genome mutation rate is relatively constant (Sanjuan et al., 2016, Lynch et al., 2016).

RNA-dependent RNA polymerases are notorious for producing mutation rates (Domingo et al., 1997). Incorporation of mutations into progeny RNA during synthesis, replication, and subgenomic transcription, coupled with the lack of a second strand for proofreading, results in the generation of a cloud of variant RNA species, often referred to as quasispecies. Such quasispecies can lead to defective genome species, as mentioned in the Background above, and these defective genome species can coinfect cells with functional virus and in some cases co-exist for years (Aaskov et al., 2006). While defective interfering genome species can also be the result of recombination, which is inhibited in this invention, mutation rates during replication and/or subgenomic expression of this invention will be dependent upon the RdRp protein. This invention presents novel safety features unique to the gene modulating single-stranded RNAi triggers and aptamers within such RNA nanostructures.

In preferred embodiments, the RNA nanostructure templates of this invention contain gene modulating single-stranded RNA that are known as “MVRNA” (referred to as MV-siRNA in original patent filing, U.S. Ser. Nos. 13/375,460 and 14/954,653 (Hauser 2010). MV-RNAs are 3-way junction RNAi triggers that have both inter-structural and sequence complementarity dependencies that upon mutation are no longer able to; 1) function as an RNAi trigger, or 2) maintain a secondary structure to reliably support an aptamer.

In preferred embodiments, the RNA nanostructure templates of this invention contain gene modulating single-stranded RNA that is MV-RNA that lose gene modulating activity upon mutation—a loss of activity fitness.

In preferred embodiments, the RNA nanostructure templates of this invention contain gene modulating single-stranded RNA that is MV-RNA that lose 3-way junction secondary structure fidelity upon mutation—a loss of replicative (encapsidation) fitness.

Encapsidation and Packaging

In virus and subviruses, the assembly of capsids or nucleocapsids is fundamental to the encapsidation and/or packaging process and, in many cases, will self-assemble in-vitro in the absence of RNA or in the presence of non-cognate RNA or polyanions. RNA virus genomes contain endogenous packaging signals to ensure efficient loading of the genome over the milieu of cellular RNA. Packaging signals can be either sequence-based as in MS or Tobacco Necrosis Virus (Ford et al., 2013), dependent upon RNA binding to a structural protein as with coronavirus (Hsieh et al., 2005), or even RNA binding to a minor non-structural protein as shown with Alphatetraviruses of insects (Mendes et al., 2016). This invention utilizes aptamer(s) as defined by (Hauser, U.S. patent application Ser. No. 16/642,244) to bind structural or non-structural proteins to assemble nucleocapsid or procapsids with the RNA nanostructure template loaded in preference over both the endogenous viral genome and milieu of cellular RNA.

In preferred embodiments, the aptamer(s) within the RNA nanostructure template of this invention bind to viral structural proteins associated with encapsidation or packaging.

In preferred embodiments, the aptamer(s) within the RNA nanostructure template of this invention bind to one or more of the viral structural protein like Nucleoprotein (FIG. 6, #3) (e.g., SARS, MERS based template) or capsid protein leading to encapsidation and/or enveloping.

In other preferred embodiments, the aptamers within the RNA nanostructure template of this invention bind to several viral proteins to elicit the formation of a minimal ribonucleoprotein matrix containing NP, VP35, VP30, and L (FIG. 6, #3) (e.g., Ebola or Marburg based template) leading to encapsidation and enveloping.

In yet other preferred embodiments, the aptamer(s) within the RNA nanostructure template of this invention bind to one or more of the viral structural (e.g., capsid) or non-structural proteins (RdRp) and form a non-enveloped particle (e.g., Tetravirus, Picornavirus, Crinivirus).

In other embodiments, the aptamer(s) within the RNA nanostructure template of this invention bind to one or more of the viral structural proteins of membrane, envelope, or transmembrane glycoprotein.

In yet other embodiments, the aptamer(s) within the nanostructure binds to viral structural proteins, and the RNA nanostructure templates of this invention contain endogenous viral packaging signal (PS) sequences.

In yet other embodiments, the aptamer(s) within the nanostructure binds to modified viral structural proteins. The RNA nanostructure templates of this invention contain engineered viral packaging signal (PS) sequences.

In still certain other embodiments, RNA nanostructure templates of this invention lack endogenous viral packaging signal (PS) sequence and are still encapsidated and/or packaged in a membrane vesicle due to the aptamers binding to a encapsidation or packing protein. For example, Coronavirus contains a PS sequence near the 3′ end of the ORF1b of ˜70 nt sequence motif within the y7 required for Nucleocapsid protein binding and packaging into the final virion (Hsieh et al., 2005) and this invention can be used to encapsidate and package the RNA nanostructure templates without endogenous packaging signals.

In further embodiments, the aptamer(s) bind to non-viral proteins and lead to additional modalities such as encapsidation or intracellular packaging or enveloping or cellular export.

The RNA nanostructure templates of this invention enable highly efficient encapsidation and/or membrane enveloping of nanostructure RNA in an intracellular setting.

In preferred embodiments, the aptamer(s) bind to 2-60 or more proteins and compete with endogenous viral proteins for intracellular packaging or enveloping.

Host Cell Viral RdRp

Ideally, this invention is subviral-dependent upon the presence of intracellular viral RdRp or RdRp-like proteins to function.

In preferred embodiments, the intracellular RdRp is present due to a viral infection of a host cell. In such a case, this invention is induced by the presence of RdRp or RdRp-like protein to elicit replication and/or subgenomic transcription of gene modulating single-stranded RNA from the RNA nanostructure template.

In certain embodiments, the replication and/or subgenomic transcription of the gene modulating single-stranded RNA templates of this invention by viral RdRp proteins reduce the presence of the genome and/or subgenomic mRNA of the host cell virus.

In preferred embodiments, the host cell RdRp viral proteins utilize the RNA nanostructure template of this invention to continuously or discontinuously, by way of subgenomic expression or replication, produce single-stranded RNAi triggers which target the host virus genome or mRNA.

In other preferred embodiments, the host cell RdRp viral proteins utilize the RNA nanostructure template of this invention to continuously or discontinuously, by way of subgenomic expression or replication, produce single-stranded RNAi triggers which target genes of the host.

In still other preferred embodiments, the host cell RdRp viral proteins utilize the RNA nanostructure template of this invention to continuously or discontinuously, by way of subgenomic expression or replication, produce single-stranded RNAi triggers which target an organism that is transkingdom.

In other embodiments, the host cell RdRp viral proteins utilize the RNA nanostructure template of this invention to continuously or discontinuously replicate the RNA nanostructure template, and the host cell degradation produces RNAi triggers which target the host virus genome or mRNA.

In preferred embodiments, this invention results in the intracellular expression of MV-RNA RNAi triggers, aptamers, or other non-coding RNAs by subgenomic expression and/or replication of the RNA nanostructure templates of this invention.

In other embodiments, this invention results in the production of one or more of MV-RNA, shRNA, pre-miRNA, sRNA, tRNA, aptamers, or other non-coding RNAs by subgenomic expression and/or replication of the RNA nanostructure templates of this invention.

In preferred embodiments, the virus and RdRp proteins useful with this invention are derived from the single-stranded (+) oriented RNA virus orders Nidovirales, Picornavirales, Tymovirales, or unassigned orders, like, but not limited to Alphatetraviridae, Bromoviridae, Caliciviridae, Flaviviridae, or Togaviridae.

In other embodiments, the virus and RdRp proteins useful with this invention are derived from the single-stranded (−) oriented RNA virus orders Mononegavirales, Bunyavirales, or unassigned orders, like, but not limited to Orthomyxoviridae (including several influenzavirus alpha, beta, gamma, delta, Isa).

In still other embodiments, the virus and RdRp proteins useful with this invention are derived from the double-stranded RNA virus unassigned orders, like, but not limited to Totivirdae, Picobirnaviridae, Partiviridae, and Endornaviridae.

Exogenous RdRp Protein

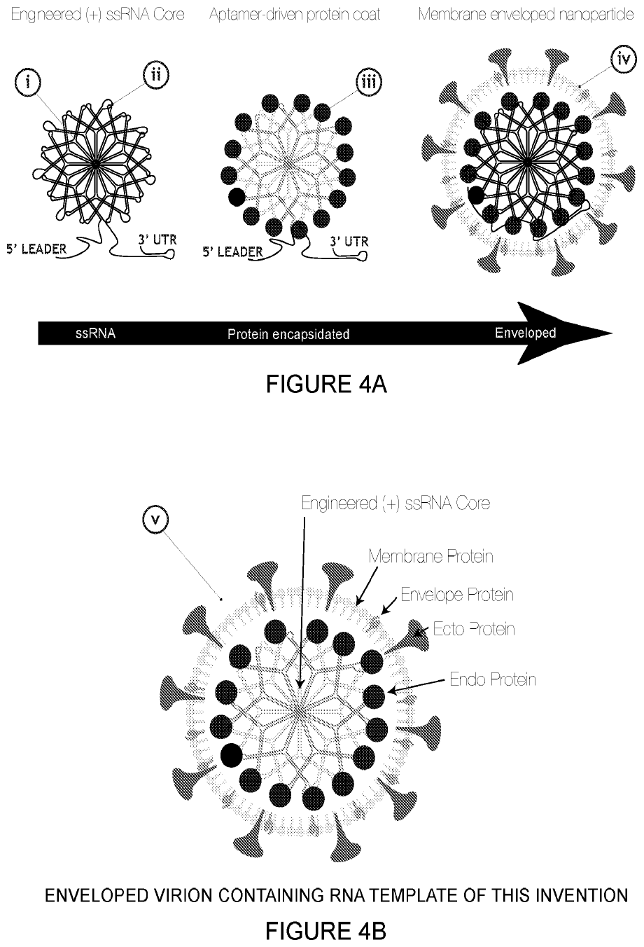

In yet other embodiments, the required RdRp or RdRp-like proteins are either attached to the RNA nanostructure by RNA aptamer sequences (FIG. 4Aiv “Endoprotein”) or contained as an mRNA within the polynucleotide sequence of the RNA nanostructure template or co-packaged in the nanoparticle as an mRNA sequence.

Exogenous RdRp mRNA

Alternatively, RdRp related proteins can be co-expressed from a flanking UTR sequence or as a “sgTranscript” (FIG. 1, 2B) of this invention or present as aptamer-bound endoproteins within a final particle or lipid nanoparticle compositions of this invention (FIG. 4).

Onco-Related TERT RdRp-Like Activity

Like the RdRp-like activity by Qβ replicase (Zamora et al., 1995), human TERT and RMRP form a distinct ribonucleoprotein complex that has RNA-dependent RNA polymerase (RdRP-LIKE) activity and produces double-stranded RNAs that can be processed into small interfering RNA in a Dicer (also known as DICER1) dependent manner (Maida et al., 2009). TERT RdRp-like activity generates short RNAs that are complementary to template RNAs and have 5′-triphosphorylated ends, which indicates de novo synthesis of the RNAs. RNA synthesis by TERT in several human carcinoma cell lines has been confirmed and found that TERT protein levels are positively correlated with RdRP activity (Maida et al., 2016).

Cancerous cells are known to have over-expression of TERT protein, which can have an RdRp-like function with this invention (FIG. 14) when the 3′ end of the nanostructure template contains the Telomerase RNA component sequence (SEQ ID NO: 49), (Maida et al., 2016).

Upon transcription by an RdRp-like protein(s), such as TERT, a helix is formed due to the binding of the transcript to the template sequence. This dsRNA can result in further transcription, translation, immune stimulation, or direct processing by RNA processing proteins (FIG. 14).

In certain non-limiting embodiments, the highly structured RNA nanostructure templates of this invention shape-shift from the spherical nanostructure (FIG. 14Ai) into a dsRNA to elicit immune stimulation and gene silencing in cancerous cells (FIG. 14Cvii).

Coronavirus Antiviral Treatment Example

The pandemic of 2020 is a reminder that societies' abilities to combat epidemics haven't much improved over the social distancing measures also practiced in 1918 during the Spanish Flu outbreak. The world's medical response methods have proven to be severely lacking. There is a significant need to develop new immediate-response treatments for this and future epidemics—over our dependence upon prophylactic medicines like vaccines as a response. Our reliance on vaccine-based medical responses has currently cost hundreds of thousands of lives in this pandemic alone. As such, this is now the single most expensive event in world history, costing the economy trillions of dollars and disrupting the livelihoods of countless people for years to come. A safe, fast, and scalable treatment is urgently needed.

Below is an example composition of a RNA nanostructure template using designs and methods of this invention, enabling a novel treatment to neutralize SARS-2 coronavirus infections and treat SARS-2 nCOV infections.

Such a novel subviral treatment would be advantageous over current methods using either antiviral or immunosuppressive pharmaceuticals, antibody, and/or vaccines as a treatment in response to a widespread and novel viral infection of a single-stranded RNA virus as occurred worldwide in 2019-2020.

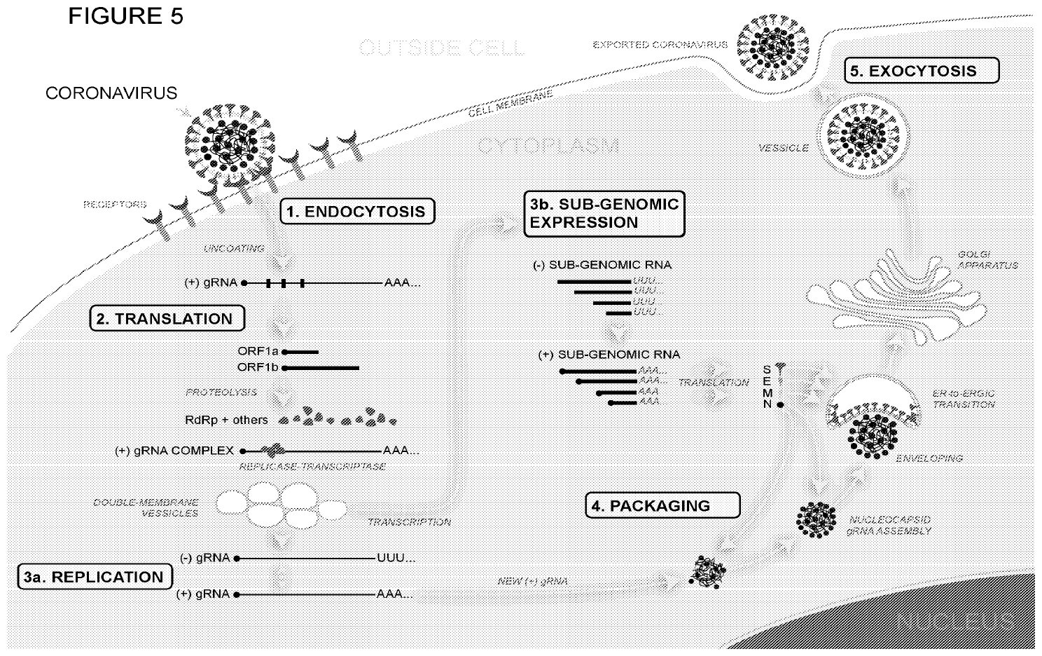

This SARS-2 treatment composition example is based on sequences;
A) SARS-2 genome (SEQ ID NO: 1),
B) a 5′ leader sequence derived from the SARS-2 genome (nucleotides 1-300) with the subgenomic promoter TRS (SEQ ID NO: 4) changed to an engineered TRS (SEQ ID NO: 5) and (nucleotides 61-63) “GTT” changed to “GCGA” to maintain the secondary structure as shown (FIG. 8H-I). (SEQ ID NO: 2),
C) the 3′ UTR from the SARS-2 genome (SEQ ID NO: 3),
D) an mCherry CDS sequence as a transfection marker (SEQ ID NO: 20 nucleotides 303 . . . 1013),
E) resulting in a template (SEQ ID NO: 6) containing (SEQ ID NO: 2, 3, 5) as a minimal construct (SEQ ID NO: 6) for inserting subgenomic transcripts (below F-I),
F) MV-RNA designed at Halo-Bio RNAi Therapeutics or Oligoengine (Seattle, Wash.) targeting SARS-2 genome and/or subviral mRNA. Examples provided as (SEQ ID NO: 7-15), but others can be designed by one skilled in the art,
G) optional MV-RNA targeting human genes for therapeutic effects (i.e., immune response genes),
H) an optional RNA aptamer (SEQ ID NO: 20 nucleotides 1030 . . . 1117) to embed in one or more loops of the MV-RNA sequences of above to elicit binding of the RNA nanostructure template to SARS-2 nucleocapsid intrans for packaging of the nanostructure template by SARS-2 coronavirus N protein. Or, such sequences can be placed in the “optional RNA features” region (FIG. 8iii) as used in this SARS-2 treatment example (SEQ ID NO: 20),
I) optional fluorogenic RNA aptamer(s) motif embedded in the RNA nanostructure templates detecting replication, and/or within the MV-RNA subgenomic transcripts to detect sgRNA expression (SEQ ID NO: 17-19),
J) resulting in a RNA nanostructure template sequence for use in as a subviral treatment of nCOV SARS-2 infections (FIG. 8, SEQ ID NO: 20) and (FIG. 5, 6, 9, 10) by making a single polynucleotide composed of B, mCherry Sequence, eTRS, F, and C. Optionally, a packaging signal (SEQ ID NO:16) can be added in RNA features (FIG. 2ii) in between mCherry CDS and eTRS in the sequence design.

In such a composition, only cells infected by Coronavirus would result in RdRp transcription of the RNA nanostructure as a template for transcriptions (FIG. 6, 9Biii, 10) and release of subvirion nanoparticles containing this invention from cells previously infected with SARS-2 coronavirus. The SARS-2 suppression activity this example of the invention is measured by immunofluorescence staining the SARS-2 spike protein present on the surface of infected cells and comparing untreated vs. treated (FIG. 10iii).

The advantages of this SARS-2 treatment example are;
1. A new method to treat viral infections in which only a subpopulation with a negative response to SARS-2 infections needs treatment vs. billions of doses required for a vaccine.
2. A new method to treat viral infections in which the treatment or cure is active only within infected hosts.
3. A new method to treat infected population clusters in which the non-infectious antiviral treatment or cure is temporarily embedded in the host's viral lifecycle and the de-novo virion output contains the treatment or cure that can further spread amongst infected hosts, until the viral outbreak is eliminated—while remaining inactive otherwise (FIG. 7).
4. Subviral nanoparticles are extremely low doses with expected TCID50 between 10⁴-10⁶ vs. 10's-100's/micrograms per dose.
5. Subviral nanoparticles are extremely scalable biological manufacturing in plants, yeast, bacteria, or other cell or cell-free systems.

Intracellular Production

This invention enables the intracellular production of RNA nanostructures through replication, subgenomic transcription, and/or packaging within a cellular environment when in the presence of RdRp and/or structural proteins from a modeled RNA virus. Such production would be typically endogenous, transgenic, or even exogenously introduced to the organism used for such intracellular production. A multitude of uses derived from intracellular replications, subgenomic transcription, and/or packaging are enabled by this invention.

In some embodiments, upon oral ingestion by a virally infected organism, the replication and/or subgenomic transcription and/or packaging of this invention is induced. Some such uses are described elsewhere in this application but include in-planta bio-pesticide production for pests, intracellular production of fungicides, or human or animal biopharmaceuticals.

In preferred embodiments, the replication and/or subgenomic transcription and/or packaging of this invention is induced by any cellular introduction of the invention to a cell infected with the modeled RNA virus.

In other embodiments, the replication and/or subgenomic transcription and/or packaging of this invention is induced by any cellular introduction of the invention to a cell that expresses recombinant RdRp proteins and/or structural proteins of a modeled RNA virus.

In certain embodiments, the RNA nanostructure templates of this invention are replicated and/or packaged within a host cell selected from a human cell or animal cell or plant cell or yeast cell or insect cell or bacterial cell, or by in-vitro transcription.

In certain specific embodiments, the RNA nanostructure templates of this invention is produced by intracellular transcription by a promoter (transgenic), virus (transient), or applied topically (exogenic) following in-vitro transcription in a general structure set forth in any one of FIG. 1 and introduced into a cell containing viral RdRp proteins or RdRp-like proteins.

Plants contain RNA-dependent RNA polymerase (RdRP) activities that synthesize short cRNAs by using cellular or viral RNAs as templates. The RNA nanostructure templates of this invention can elicit synthesis by in-planta RdRp despite lacking a viral origin.

In certain specific embodiments, the RNA nanostructure templates of this invention are replicated and/or elicit subgenomic expression and/or are further packaged into coated enveloped or non-enveloped nanoparticles by in-planta RdRp's.

In certain other embodiments, the plant is deficient in endogenous RdRp, and the RNA nanostructure templates of this invention are replicated and/or elicit subgenomic expression, and/or are further packaged into coated enveloped or nonenveloped nanoparticles in-planta by viral RdRp's.

While plants have been approved by the FDA for the hydroponic production and encapsulation of protein-based drugs, in planta production of subviral RNA nanoparticles with replicating or subgenomic expressing RNA encapsidated with programmable surface characteristics for pharmaceutical use has not been shown in the art. The methods of this invention provide a platform in which future RNA subviral nanoparticle drug production with replication and subgenomic expression properties can be accomplished intracellularly at scale.

Plants offer an ideal alternative to conventional manufacturing systems. Plants are not hosts for human pathogens. The lignin and cellulose packed plant cell wall provides a general natural protection for RNA nanostructures for human use because humans are incapable of breaking down the glycosidic bonds of the plant cell wall. In humans, gut bacteria digest the plant cell wall and release its contents into the gut lumen.

Also, plant cells have a similar capacity as mammalian cells to produce protein drugs and could be used to also produce RNA nanostructures for replicating and subgenomic expressing drugs by utilizing the methods of this invention. Protein-based drug production has been shown in tobacco plants and carrot cell suspension cultures and, without limits, can be used to produce the drugs utilizing the methods of this invention.

Similar to mammalian, insect, fungal, and bacterial cells, plant cells can fundamentally facilitate expression, folding, and the self-replicating of RNA-based structures. Plants stably transformed with transgenes designed using the methods of this invention can be easily propagated from seeds. Agrobacterium tumefaciens is used to deliver such transgenes to the nucleus, whereas a particle delivery system is used to transform plants that are recalcitrant to Agrobacteria-mediated transformation.

Method of Crop Protection

Food security has been a concern for many years, and one of the principal causes of loss of food has been preharvest destruction by pathogens and pests. Successful control of these has been possible with the use of chemicals; however, it is now recognized that chemical residues left behind on the crop may be harmful to the consumer. This provided an impetus to search for alternative means of controlling pathogens and pests, especially methods relying on biological agents or their products.

Upon oral ingestion, RNA nanostructures in plant cells are generally protected from the stomach from acids and enzymes but are subsequently released into the gut lumen by microbes in humans and animals that digest the plant cell wall. In some insect pests, such as certain Hemiptera and Lepidoptera, plant cell degrading enzymes are present in the saliva, and only a small amount of protein or lipid encased RNA makes it into gut cells (i.e., viruses). This invention enables the amplification of this small amount of RNA delivered into the gut cells of target insects and dramatically increases the activity of the RNA.

The large mucosal area of the target organism intestine offers an ideal system for oral nanoparticle-based drug delivery and is also the common location for viral vector infection (i.e. DENV, tetravirus, DvvV1-3, Providence virus). When certain nanoparticle surfaces such as viral coat proteins, receptor-binding peptides, cell-penetrating peptides, endosomal peptides, are used as a RNA coatings, organism and cellular specificity can be achieved, but only a limited amount of RNA penetrates the gut cell membrane to elicit activity in the cytoplasm.

The compositions and methods for RNA nanostructure templates of this invention provide dramatic amplification of the single-stranded RNA in the cytoplasm when in the presence of RdRp. The biopesticidal activity is significantly increased due to the subgenomic expression of the gene modulating single-stranded RNA within the template and/or the replication of single-stranded RNA of the RNA nanostructure template and further degradation of the newly synthesized single-stranded RNA into small active gene modulators. This is in contrast with prior methods using virus as a source for RNA interference in that prior methods rely on long dsRNA viral intermediates as the source for RNAi triggers. This invention provides a significant advantage of potency since the molar ratio of single-stranded RNA produced by RdRp is significantly higher than the production of dsRNA intermediates during replication and that the active RNA employed in this invention are solely single-stranded RNA derived from a different point in the viral lifecycle.

In some embodiments, the RNA nanostructure templates of this invention have useful purposes of crop protection of plants suffering from viral infection, fungal infection or insect infestation as a biopesticide, or even the removal of unwanted weed/plant pests from crops as a bioherbicide when comprising the step of:

administering to a pest thereof an effective amount of the polynucleotide, wherein said polynucleotide is a single-stranded RNA represented by RNA nanostructure template described in this invention, encapsidated by protein as a virion-like particle or enveloped by lipidic proteins as a virion-like particle or formulated with an acceptable formulation excipient at a dose sufficient to reduce the fitness of the target pest.

In preferred embodiments, an effective amount is less than twenty parts-per-million (20 ppm), five parts-per-million (5 ppm), two parts-per-million (2 ppm) or lower.

In preferred embodiments, the RNA nanostructure template of this invention has gene modulating single-stranded RNA within the template designed to target an insect gene (FIG. 11-13A) and reduces fitness of the insect by suppressing one or more insect genes.

In other preferred embodiments, the RNA nanostructure template of this invention has gene modulating single-stranded RNA within the template designed to target a pest (FIG. 11-13A) and reduces fitness of the pest by suppressing one or more genes of the pest.

In preferred embodiments, an effective amount is determined by exposure and/or ingestion of the RNA nanostructure template alone or formulated with excipients or encapsidated in protein as a virion-like nanoparticle or further enveloped by lipidic proteins as a virion-like particle in an amount resulting in stunting and/or death of the target pest.

In non-limiting embodiments, the RNA nanostructure template of this invention is packaged into the structural protein(s) of a modeled RNA virus of the pest host to match host tropism with a modeled RNA virus, provide degradation protection (FIG. 13A, #1-3) of the RNA nanostructure template, and delivery the RNA nanostructure template into the cytoplasm of the pest (FIG. 13),

In such preferred embodiments, the RNA nanostructure template is non-infections in that it does not code for the structural protein in which it is encapsidated (FIG. 13A, #4).

In non-limiting embodiments, the RNA nanostructure template of this invention has been modeled from a positive (+) strand single-stranded Dicistrovirus DVv3 (SEQ ID NO: 32, Sijun L et al., 2017) persistently infecting Western Corn Rootworm, and the gene modulating single-stranded RNA within the template are designed to modulate gene of Western Corn Rootworm upon exposure or ingestion (FIG. 13B) and lead to significant stunting and death.

Below is an example composition of an RNA nanostructure template using designs and methods of this invention enabling a novel subviral biopesticide targeting Western Corn Rootworm (WCR), an insect that causes greater than $1 Billion dollars of damage to Corn crops annually and that is emerging with acquired resistance to dsRNA-based RNAi.

Such a novel subviral RNAi biopesticide treatment is advantageous over current RNAi methods as it provides an endogenous and alternative uptake route into the insect gut cells upon ingestion based on a natural and persistent virus of the host, is non-infectious, and will result in a systemic molar amplification of the single-stranded RNAi triggers by viral RdRp upon cell entry.

This WCR biopesticide composition example is based on sequences;
A) Viral DVVv3 genome (SEQ ID NO: 35), with the structural protein ORF2 (nucleotides 9235-12,456) to be removed.
B) a 5′ leader sequence derived from nucleotides 1-675 of the DVVv3 genome (SEQ ID NO: 36),
C) the 3′ of the DVVv3 genome (SEQ ID NO: 37),
D) an RdRp coding sequence (SEQ ID NO: 35 nucleotides 7420 . . . 9066) or entire ORF1 (SEQ ID NO: 35 nucleotides 676-9189),
E) a subviral intergenic region (IGR) from (SEQ ID NO: 35 nucleotides 9,193-9,234) of the DVVv3 genome or (SEQ ID NO: 38),
F) a central RNA region containing ˜16 MV-RNA designed at Halo-Bio RNAi Therapeutics or Oligoengine (Seattle, Wash.) to genes of four different WCR genes. Every 4th MV-RNA is adjoined with a 3-way junction RNA containing Mango aptamers (SEQ ID NO: 18) on the loops for tracking of the transcript (FIG. 11Av-11Biv, SEQ ID NO: 41),
G) an optional sequence containing a collection of GFP-like fluorescent aptamer sequences to append to the 3 ′end of the central RNA region (SEQ ID NO: 40),

And multimodal biopesticides for WCR can be derived from a minimal template (SEQ ID NO: 42) using the <B> 5′ region, <E> IGR, and <C> 3′ region with <G> fluorescent green aptamers to make a polynucleotide made up of <B><E><G><C> and then the central region with the 16 MV-RNA <F> (SEQ ID NO: 41) to target Western Corn Rootworm (WCR) genes vATPase (SEQ ID NO: 45), Dvsnf7 (SEQ ID NO: 46), Sec23 (SEQ ID NO: 47), Snakeskin (SEQ ID NO: 48) made up of <B><E><F><G><C> (SEQ ID NO: 48) as a minimal template targeting WCR or assembled for;
Infection-dependent amplification: by producing an RNA polynucleotide made up of <B><F><optional G><C> without subviral activity,
Infection-dependent amplification with subviral expression: by producing an RNA polynucleotide made up of <B><E><F><optional G><C>,

In such a composition above, only cells infected by DVVv3 virus would result in RdRp transcription of the RNA nanostructure as a template for transcriptions as shown in construct example (FIG. 11Aiii) and the maintained fluorescent single of the RNA in the presence of DVVv3 RdRp (FIG. 12Ai).

Other multimodal biopesticides for WCR can be assembled for self-amplification and/or subgenomic expression by inclusion of RdRp and related proteins for the DVVv3 genome ORF1 (SEQ ID NO: 35 nucleotides 676 . . . 9189) or RdRp itself (SEQ ID NO:35 nucleotides 7420 . . . 9066);
Self-amplification: by producing an RNA polynucleotide made up of <B><D><F><optional G><C>,
Self-amplification and subviral expression: by producing an RNA polynucleotide made up of <B><D><E><F><optional G><C> (SEQ ID NO: 44),

In such a composition, gut cells that receive the RNA nanostructure template would produce the RdRp machinery for transcription of the RNA nanostructure template, and the RNA would be non-infectious. RNA transcription can be monitored by the inclusion of the optional fluorescent aptamers (SEQ ID NO: 40), and examples are provided for in-vitro (FIG. 12Ai) and in-vivo (FIG. 12Biii) activity.

Bloodsucking insects are vectors for diseases in humans and animals. These insects ingest disease-producing microorganisms during a blood meal from an infected host (human or animal) and later transmit it into a new host, after the pathogen has replicated. Often, once a vector becomes infectious, they are capable of transmitting the pathogen for the rest of their life during each subsequent bite/blood meal.

Vector-borne diseases are human illnesses caused by parasites, viruses, and bacteria that are transmitted by vectors. Every year there are more than 700,000 deaths from diseases such as malaria, dengue, schistosomiasis, human African trypanosomiasis, leishmaniasis, Chagas disease, yellow fever, Japanese encephalitis, and onchocerciasis (WHO website, http://who.int). The RNA nanostructure templates of this invention can be used to target disease vectoring insects.

In some embodiments, the RNA nanostructure templates of this invention contain gene modulating single-stranded RNA within the template that is designed to target an insect infected with a virus (i.e., mosquitos infected with Dengue).

In other embodiments, the RNA nanostructure templates of this invention contain gene modulating single-stranded RNA within the template that is designed to target an insect and the template also contains a coding sequence for a protein.

In other embodiments, the RNA nanostructure templates of this invention are replicated by the RdRp's of a parasitic virus, and the gene modulating single-stranded RNA within the template is designed to targeting the insect vectoring the parasite.

In specific non-limiting embodiments, the viral RdRp is from Matryoshka RNA Virus 1 (Charon J., et al., 2019) and the parasite is Plasmodium.

In other specific embodiments, the RNA nanostructure templates of this invention are replicated by the RdRp's of a parasitic virus, and the gene modulating single-stranded RNA within the template is designed to targeting the parasite infecting the host. The template nanostructure contains a coding sequence for a protein.

In specific non-limiting embodiments, the protein encoded within the parasite by the RNA nanostructure method of this invention is an argonaut protein (Hentzschel et al., 2020).

Viral targets of the invention include, but are not limited to, viruses or viroids, bacteria, insects, nematodes, fungi, parasites, and the like. Viruses include any plant, animal, or human virus, for example, Group III, IV, V ssRNA virus like SARS or MERS coronavirus, west Nile virus, dengue virus, rhinovirus, or flavivirus, tetravirus, picornavirus, bromovirus, sindbis virus, tobacco or cucumber mosaic virus, HIV, HBV, HSV, HPV, ringspot virus, necrosis virus, maize dwarf mosaic virus, etc. Specific fungal and viral pathogens for the major crops include: Soybeans: Phytophthora megasperma f.sp. glycinea, Macrophomina phaseolina, Rhizoctonia solani, Selerotinia sclerotiorum, Fusarium oxysporum, Diaporthe phaseolorum var. sojae (Phomopsis sojae), Diaporthe phaseolorum var. caulivora, Sclerotium rolfsii, Cercospora kikuchii, Cercospora sojina, Peronospora manshurica, Colletotrichum dematium (Colletotrichum truncatum), Corynespora cassiicola, Septoria glycines, Phyllosticta sojicola, Alternaria alternata, Pseudomonas syringae p.v. glycinea, Xanthomonas campestris p.v. phaseoli, Microsphaera diffusa, Fusarium semitectum, Phialophora gregata, Soybean mosaic virus, Glomerella glycines, Tobacco Ring spot virus, Tobacco Streak virus, Phakopsora pachyrhizi, Pythium aphanidermatum, Pythium ultimum, Pythium debaryanum, Tomato spotted wilt virus, Heterodera glycines, Fusarium solani; Canola: Albugo candida, Alternaria brassicae, Leptosphaeria maculans, Rhizoctonia solani, Sclerotinia sclerotiorum, Mycosphaerella brassiccola, Pythium ultimum, Peronospora parasitica, Fusarium roseum, Alternaria alternata; Alfalfa: Clavibacter Michigan's subsp. insidiosum, Pythium ultimum, Pythium irregulare, Pythium splendens, Pythium debaryanum, Pythium aphanidermatum, Phytophthora megasperma, Peronospora trifoliorum, Phoma medicaginis var. medicaginis, Cercospora medicaginis, Pseudopeziza medicaginis, Leptotrochila medicaginis, Fusarium spp., Xanthomonas campestris p.v. alfalfae, Aphanomyces euteiches, Stemphylium herbarum, Stemphylium alfalfae; Wheat: Pseudomonas syringae p.v. atrofaciens, Urocystis agropyri, Xanthomonas campestris p.v. translucens, Pseudomonas syringae p.v. syringae, Alternaria alternata, Cladosporium herbarum, Fusarium graminearum, Fusarium avenaceum, Fusarium culmorum, Ustilago tritici, Ascochyta tritici, Cephalosporium gramineum, Collotetrichum graminicola, Erysiphe graminis f.sp. tritici, Puccinia graminis f.sp. tritici, Puccinia recondita f.sp. tritici, Puccinia striiformis, Pyrenophora tritici-repentis, Septoria nodorum, Septoria tritici, Septoria avenae, Pseudocercosporella herpotrichoides, Rhizoctonia solani, Rhizoctonia cerealis, Gaeumannomyces graminis var. tritici, Pythium aphanidermatum, Pythium arrhenomanes, Pythium ultimum, Bipolaris sorokiniana, Barley Yellow Dwarf Virus, Brome Mosaic Virus, Soil Borne Wheat Mosaic Virus, Wheat Streak Mosaic Virus, Wheat Spindle Streak Virus, American Wheat Striate Virus, Claviceps purpurea, Tilletia tritici, Tilletia laevis, Tilletia indica, Pythium gramicola, High Plains Virus, European wheat striate virus; Sunflower: Plasmophora halstedii, Sclerotinia sclerotiorum, Aster Yellows, Septoria helianthi, Phomopsis helianthi, Alternaria helianthi, Alternaria zinniae, Botrytis cinerea, Phoma macdonaldii, Macrophomina phaseolina, Erysiphe cichoracearum, Rhizopus oryzae, Rhizopus arrhizus, Rhizopus stolonifer, Puccinia helianthi, Verticillium dahliae, Erwinia carotovorum p.v. carotovora, Cephalosporium acremonium, Phytophthora cryptogea, Albugo tragopogonis; Corn: Fusarium moniliforme var. subglutinans, Erwinia stewartii, Fusarium moniliforme, Gibberella zeae (Fusarium graminearum), Stenocarpella maydis (Diplodia maydis), Pythium irregulare, Pythium debaryanum, Pythium graminicola, Pythium splendens, Pythium ultimum, Pythium aphanidermatum, Aspergillus flavus, Bipolaris maydis O, T (Cochliobolus heterostrophus), Helminthosporium carbonum I, II & III (Cochliobolus carbonum), Exserohilum turcicum I, II & III, Helminthosporium pedicellatum, Physoderma maydis, Phyllosticta maydis, Kabatiella maydis, Cercospora sorghi, Ustilago maydis, Puccinia sorghi, Puccinia polysora, Macrophomina phaseolina, Penicillium oxalicum, Nigrospora oryzae, Cladosporium herbarum, Curvularia lunata, Curvularia inaequalis, Curvularia pallescens, Clavibacter michiganense subsp. nebraskense, Trichoderma viride, Maize Dwarf Mosaic Virus A & B, Wheat Streak Mosaic Virus, Maize Chlorotic Dwarf Virus, Claviceps sorghi, Pseudomonas avenae, Erwinia chrysanthemi p.v. zea, Erwinia carotovora, Corn stunt spiroplasma, Diplodia macrospora, Sclerophthora macrospora, Peronosclerospora sorghi, Peronosclerospora philippinensis, Peronosclerospora maydis, Peronosclerospora sacchari, Sphacelotheca reiliana, Physopella zeae, Cephalosporium maydis, Cephalosporium acremonium, Maize Chlorotic Mottle Virus, High Plains Virus, Maize Mosaic Virus, Maize Rayado Fino Virus, Maize Streak Virus, Maize Stripe Virus, Maize Rough Dwarf Virus; Sorghum: Exserohilum turcicum, Colletotrichum graminicola (Glomerella graminicola), Cercospora sorghi, Gloeocercospora sorghi, Ascochyta sorghina, Pseudomonas syringae p.v. syringae, Xanthomonas campestris p.v. holcicola, Pseudomonas andropogonis, Puccinia purpurea, Macrophomina phaseolina, Periconia circinata, Fusarium moniliforme, Alternaria alternata, Bipolaris sorghicola, Helminthosporium sorghicola, Curvularia lunata, Phoma insidiosa, Pseudomonas avenae (Pseudomonas alboprecipitans), Ramulispora sorghi, Ramulispora sorghicola, Phyllachara sacchari, Sporisorium reilianum (Sphacelotheca reiliana), Sphacelotheca cruenta, Sporisorium sorghi, Sugarcane mosaic H, Maize Dwarf Mosaic Virus A & B, Claviceps sorghi, Rhizoctonia solani, Acremonium strictum, Sclerophthona macrospora, Peronosclerospora sorghi, Peronosclerospora philippinensis, Sclerospora graminicola, Fusarium graminearum, Fusarium oxysporum, Pythium arrhenomanes, Pythium graminicola, etc.

Nematodes include parasitic nematodes such as root-knot, cyst, and lesion nematodes, including Heterodera and Globodera spp.; particularly Globodera rostochiensis and Globodera pailida (potato cyst nematodes); Heterodera glycines (soybean cyst nematode); Heterodera schachtii (beet cyst nematode); and Heterodera avenae (cereal cyst nematode). Additional nematodes include: Heterodera cajani; Heterodera trifolii; Heterodera oryzae; Globodera tabacum; Meloidogyne incognita; Meloidogyne javonica; Meloidogyne hapla; Meloidogyne arenaria; Meloidogyne naasi; Meloidogyne exigua; Xiphinema index; Xiphinema italiae; Xiphinema americanum; Xiphinema diversicaudatum; Pratylenchus penetrans; Pratylenchus brachyurus; Pratylenchus zeae; Pratylenchus coffeae; Pratylenchus thornei; Pratylenchus scribneri; Pratylenchus vulnus; Pratylenchus curvitatus; Radopholus similis; Radopholus citrophilus; Ditylenchus dipsaci; Helicotylenchus multicintus; Rotylenchulus reniformis; Belonolaimus spp.; Paratrichodorus anemones; Trichodorus spp.; Primitivus spp.; Anguina tritici; Bider avenae; Subanguina radicicola; Tylenchorhynchus spp.; Haplolaimus seinhorsti; Tylenchulus semipenetrans; Hemicycliophora arenaria; Belonolaimus langicaudatus; Paratrichodorus xiphinema; Paratrichodorus christiei; Rhadinaphelenchus cocophilus; Paratrichodorus minor; Hoplolaimus galeatus; Hoplolaimus columbus; Criconemella spp.; Paratylenchus spp.; Nacoabbus aberrans; Aphelenchoides besseyi; Ditylenchus angustus; Hirchmaniella spp.; Scutellonema spp.; Hemicriconemoides kanayaensis; Tylenchorynchus claytoni; and Cacopaurus pestis.

Insect pests include insects selected from the orders Coleoptera, Diptera, Hymenoptera, Lepidoptera, Mallophaga, Homoptera, Hemiptera, Orthoptera, Thysanoptera, Dermaptera, Isoptera, Anoplura, Siphonaptera, Trichoptera, etc., particularly Coleoptera and Lepidoptera. Insect pests of the invention for the major crops include: Maize: Ostrinia nubilalis, European corn borer; Agrotis ipsilon, black cutworm; Helicoverpa zea, corn earworm; Spodoptera frugiperda, fall armyworm; Diatraea grandiosella, southwestern corn borer; Elasmopalpus lignosellus, lesser cornstalk borer; Diatraea saccharalis, sugarcane borer; Diabrotica virgifera, western corn rootworm; Diabrotica longicornis barberi, northern corn rootworm; Diabrotica undecimpunctata howardi, southern corn rootworm; Melanotus spp., wireworns; Cyclocephala borealis, northern masked chafer (white grub); Cyclocephala immaculata, southern masked chafer (white grub); Popillia japonica, Japanese beetle; Chaetocnema pulicaria, corn flea beetle; Sphenophorus maidis, maize billbug; Rhopalosiphum maidis, corn leaf aphid; Anuraphis maidiradicis, corn root aphid; Blissus leucopterus leucopterus, chinch bug; Melanoplus femurrubrum, redlegged grasshopper; Melanoplus sanguinipes, migratory grasshopper; Hylemya platura, seedcorn maggot; Agromyza parvicornis, corn blotch leafminer; Anaphothrips obscrurus, grass thrips; Solenopsis milesta, thief ant; Tetranychus urticae, twospotted spider mite; Sorghum: Chilo partellus, sorghum borer; Spodoptera frugiperda, fall armyworn; Helicoverpa zea, corn earworm; Elasmopalpus lignosellus, lesser cornstalk borer; Feltia subterranea, granulate cutworm; Phyllophaga crinita, white grub; Eleodes, Conoderus, and Aeolus spp., wireworms; Oulema melanopus, cereal leaf beetle; Chaetocnema pulicaria, corn flea beetle; Sphenophorus maidis, maize bilibug; Rhopalosiphum maidis; corn leaf aphid; Sipha flava, yellow sugarcane aphid; Blissus leucopterus leucopterus, chinch bug; Contarinia sorghicola, sorghum midge; Tetranychus cinnabarinus, carmine spider mite; Tetranychus urticae, twospotted spider mite; Wheat: Pseudaletia unipunctata, army worn; Spodoptera frugiperda, fall armyworm; Elasmopalpus lignosellus, lesser cornstalk borer; Agrotis orthogonia, western cutworm; Elasmopalpus lignosellus, lesser cornstalk borer; Oulema melanopus, cereal leaf beetle; Hypera punctata, clover leaf weevil; Diabrotica undecimpunctata howardi, southern corn rootworm; Russian wheat aphid; Schizaphis graminum, greenbug; Macrosiphum avenae, English grain aphid; Melanoplus femurrubrum, redlegged grasshopper; Melanoplus differentialis, differential grasshopper; Melanoplus sanguinipes, migratory grasshopper; Mayetiola destructor, Hessian fly; Sitodiplosis mosellana, wheat midge; Meromyza americana, wheat stem maggot; Hylemya coarctata, wheat bulb fly; Frankliniella fusca, tobacco thrips; Cephus cinctus, wheat stem sawfly; Aceria tulipae, wheat curl mite; Sunflower: Suleima helianthana, sunflower bud moth; Homoeosoma electellum, sunflower moth; Zygogramma exclamationis, sunflower beetle; Bothyrus gibbosus, carrot beetle; Neolasioptera murtfeldtiana, sunflower seed midge; Cotton: Heliothis virescens, cotton budworm; Helicoverpa zea, cotton bollworm; Spodoptera exigua, beet armyworm; Pectinophora gossypiella, pink bollworm; Anthonomus grandis, boll weevil; Aphis gossypii, cotton aphid; Pseudatomoscelis seriatus, cotton fleahopper; Trialeurodes abutilonea, bandedwinged whitefly; Lygus lineolaris, tarnished plant bug; Melanoplus femurrubrum, redlegged grasshopper; Melanoplus differentialis, differential grasshopper; Thrips tabaci, onion thrips; Franklinkiella fusca, tobacco thrips; Tetranychus cinnabarinus, carmine spider mite; Tetranychus urticae, twospotted spider mite; Rice: Diatraea saccharalis, sugarcane borer; Spodoptera frugiperda, fall armyworm; Helicoverpa zea, corn earworm; Colaspis brunnea, grape colaspis; Lissorhoptrus oryzophilus, rice water weevil; Sitophilus oryzae, rice weevil; Nephotettix nigropictus, rice leafhopper; Blissus leucopterus leucopterus, chinch bug; Acrosternum hilare, green stink bug; Soybean: Pseudoplusia includens, soybean looper; Anticarsia gemmatalis, velvetbean caterpillar; Plathypena scabra, green cloverworm; Ostrinia nubilalis, European corn borer; Agrotis ipsilon, black cutworm; Spodoptera exigua, beet armyworm; Heliothis virescens, cotton budworm; Helicoverpa zea, cotton bollworm; Epilachna varivestis, Mexican bean beetle; Myzus persicae, green peach aphid; Empoasca fabae, potato leafhopper; Acrosternum hilare, green stink bug; Melanoplus femurrubrum, redlegged grasshopper; Melanoplus differentialis, differential grasshopper; Hylemya platura, seedcorn maggot; Sericothrips variabilis, soybean thrips; Thrips tabaci, onion thrips; Tetranychus turkestani, strawberry spider mite; Tetranychus urticae, twospotted spider mite; Barley: Ostrinia nubilalis, European corn borer; Agrotis ipsilon, black cutworm; Schizaphis graminum, greenbug; Blissus leucopterus leucopterus, chinch bug; Acrosternum hilare, green stink bug; Euschistus servus, brown stink bug; Delia platura, seedcorn maggot; Mayetiola destructor, Hessian fly; Petrobia latens, brown wheat mite; Oil Seed Rape: Brevicoryne brassicae, cabbage aphid; Phyllotreta cruciferae, Flea beetle; Mamestra configurata, Bertha armyworm; Plutella xylostella, Diamond-back moth; Delia spp., Root maggots.

Dengue Virus Biopesticide Example

Aedes aegypti is the vector of some of the most important vector-borne diseases like dengue, chikungunya, zika, and yellow fever, affecting millions of people worldwide. The cellular processes that follow a blood meal in the mosquito midgut are directly associated with pathogen transmission.

Dengue virus (DENV) is a member of the Flavivirus genus of the Flaviviridae family of enveloped, positive-strand RNA viruses. The Flavivirus genus includes viruses transmitted by mosquitoes and ticks, as well as zoonotic agents with no known arthropod vector. The dengue viruses are comprised of four distinct serotypes, DENV1 through DENV4, which are transmitted to humans through the bites of two mosquito species: Aedes aegypti and A. albopictus (Clyde et al., 2006).

In a similar manner to the Coronavirus antiviral example above, this invention can also be used with gene modulating single-stranded RNA that targets the host and the virus to treat the host and eliminate the infection or target the host alone for a disease-inducible secondary effect, like organism-specific lethality. In this example use, the subviral nanoparticle is used to selectively kill Aedes aegypti (mosquitos) that are infected with Dengue Virus by using DENV RdRp to produce gene modulating single-stranded RNA in the mosquito gut cells to reduce viral titer (Xi et al., 2008) and/or to reduce fitness of the specific and infected insect host that would have vectored the disease to other organisms by suppressing Chitin Synthase (Lopez et. al., 2019).

This DENV composition contains;
A) Dengue Virus Genome (SEQ ID NO: 21)
B) a 5′ leader from the DENV genome (SEQ ID NO: 22),
C) a 3′ UTR from the DENV genome (SEQ ID NO: 23),
D) a DENV 5′ cyclization sequence (SEQ ID NO: 22),
E) a DENV 3 cyclization sequence (SEQ ID NO: 23),
F) an engineered 5′ cyclization sequence (SEQ ID NO: 26),
G) an engineered 3′ cyclization sequence (SEQ ID NO: 27)
H) MV-RNA (SEQ ID NO: 28-32) targeting Aedes aegypti Chitin Synthase mRNA, Accession AF223577 (SEQ ID NO: 50) as a central region (SEQ ID NO: 33),
H) and a single-stranded polynucleotide composed of <B><H><C> or more preferably <B with D mutated to F><H><C with E mutated to G> resulting in an RNA nanostructure template sequence for use as a biopesticide targeting DENV-infected mosquitos (SEQ ID NO: 34).

In such a composition, only the genes of Aedes aegypti infected by DENV would be targeted. The resulting amplification of RNAi to critical genes within these infected cells would trigger death.

The advantages are;
1. An extremely low concentration can be used to selectively kill DENV infected mosquitoes.
2. Subviral nanoparticles are extremely scalable biological manufacturing in plants, yeast, bacteria, or other cell or cell-free systems.
3. The subviral nanoparticles themselves are not infectious and pose no risk to non-target organisms.
4. The subviral nanoparticle replication is transient for several passages and does not replicate indefinitely.

In addition to DENV, flaviviruses that are significant threats to human health include yellow fever virus, West Nile virus (WNV), Japanese encephalitis virus, and tick-borne encephalitis virus. This invention can be applied to each of these flaviviruses using the same methodologies of this example.

Methods of Regulating Gene Expression

A target gene may be a known gene target, or, alternatively, a target gene may be not known, i.e., a random sequence may be used. In certain embodiments, target mRNA levels of one or more, preferably two or more, target mRNAs are reduced at least 10%, at least 20%, at least 30%, at least 40%, at least 50%, at least 60%, at least 70%, at least 75%, at least 80%, at least 90%, or at least 95%.

In one embodiment of the invention, the level of inhibition of target gene expression (i.e., mRNA expression) is at least 90%, at least 95%, at least 98%, at least 99% or is almost 100%, and hence the cell or organism will in effect have the phenotype equivalent to a so-called “knock out” of a gene. However, in some embodiments, it may be preferred to achieve only partial inhibition so that the phenotype is equivalent to a so-called “knockdown” of the gene. This method of knocking down gene expression can be used therapeutically or for research (e.g., to generate models of disease states, to examine the function of a gene, to assess whether an agent acts on a gene, to validate targets for drug discovery).

In certain embodiments, the nanostructure for replication and subgenomic expression using the compositions and methods of this present invention is synthesized as RNA, using techniques widely available in the art, then introduced directly into virally infected or non-infected cells.

In other embodiments, it is expressed in vitro or in vivo using appropriate and widely known techniques, then formulated in the same setting. Accordingly, in certain embodiments, the present invention includes in vitro and in vivo expression vectors or sequences comprising the sequence of a nanostructure for replication and subgenomic expression. Methods well known to those skilled in the art may be used to construct expression vectors containing sequences encoding a nanostructure for replication and subgenomic expression, structural and non-structural proteins, as well as appropriate transcriptional and translational control elements. These methods include in vitro recombinant DNA techniques, synthetic techniques, and in vivo genetic recombination. Such techniques are described, for example, in Sambrook, J. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York, N.Y.

In various embodiments, a target gene is a gene derived from the cell into which an RNA nanostructure for replication and subgenomic expression is to be introduced, an endogenous gene, an exogenous gene, a transgene, or a gene of a pathogen that is present in the cell after transfection thereof. Depending on the particular target gene and the amount of the RNA nanostructure for replication and subgenomic expression delivered into the cell, the method of this invention may cause partial or complete inhibition of the expression of the target gene. The cell containing the target gene may be derived from or contained in any organism (e.g., plant, animal, protozoan, virus, bacterium, or fungus).

Inhibition of the expression of the target gene can be verified by means including, but not limited to, observing or detecting an absence or observable decrease in the level of the protein encoded by a target gene, and/or mRNA product from a target gene, and/or a phenotype associated with expression of the gene, using techniques known to a person skilled in the field of the present invention.

Examples of cell characteristics that may be examined to determine the effect caused by the introduction of an RNA nanostructure for replication and subgenomic expression of the invention include cell growth, apoptosis, cell cycle characteristics, cellular differentiation and morphology, qRT-PCR, and fluorogenicity.

A nanostructure for replication and subgenomic expression may be directly introduced to the cell (i.e., intracellularly), or introduced extracellularly into a cavity, interstitial space, into the circulation of an organism, introduced orally, by ingestion of the expression host, by bathing an organism in a solution containing the RNA nanostructure for replication and subgenomic expression, or by some other means sufficient to deliver the RNA nanostructure for replication and subgenomic expression into the cell.

In addition, a vector engineered to express a nanostructure for replication and subgenomic expression may be introduced into a cell, wherein the vector expresses the nanostructure for replication and subgenomic expression, thereby introducing it into the cell. Methods of transferring an expression vector into a cell are widely known and available in the art, including, e.g., transfection, lipofection, scrape loading, electroporation, microinjection, infection, gene gun, and retrotransposition. Generally, a suitable method of introducing a vector into a cell is readily determined by one of skill in the art based upon the type of vector and the type of cell, and teachings widely available in the art. Infective agents may be introduced by a variety of means readily available in the art, including, e.g., nasal inhalation.

Methods of inhibiting gene expression using self-replicating moiety-coated RNA nanostructures of the invention may be combined with other knockdown and knockout methods, e.g., gene targeting, antisense RNA, ribozymes, double-stranded RNA (e.g., shRNA and siRNA) to further reduce expression of a target gene.

In different embodiments, target cells of the invention are primary cells, cell lines, immortalized cells, or transformed cells. A target cell may be a somatic cell or a germ cell. The target cell may be a non-dividing cell, such as a neuron, or it may be capable of proliferating in vitro in suitable cell culture conditions. Target cells may be normal cells, or they may be diseased cells, including those containing a known genetic mutation. Eukaryotic target cells of the invention include mammalian cells, such as, for example, a human cell, a murine cell, a rodent cell, and a primate cell. In one embodiment, a target cell of the invention is a stem cell, which includes, for example, an embryonic stem cell, such as a murine embryonic stem cell.

The nanostructures for replication and subgenomic expression and methods of the present invention may be used for regulating genes in plants, e.g., by providing RNA for systemic or non-systemic regulation of genes.

The nanostructures for replication and subgenomic expression and methods of the present invention are useful for regulating endogenous genes of a plant pest or pathogen.

The nanostructures for replication and subgenomic expression and methods of the present invention may be used to treat any of a wide variety of diseases or disorders, including, but not limited to, inflammatory diseases, cardiovascular diseases, nervous system diseases, tumors, demyelinating diseases, digestive system diseases, endocrine system diseases, reproductive system diseases, hemic and lymphatic diseases, immunological diseases, mental disorders, musculoskeletal diseases, neurological diseases, neuromuscular diseases, metabolic diseases, sexually transmitted diseases, skin and connective tissue diseases, urological diseases, and infections.

In certain embodiments, the methods are practiced on an animal, in particular embodiments, a mammal, and in certain embodiments, a human.

Accordingly, in one embodiment, the present invention includes methods of using nanostructures for replication and subgenomic expression for the treatment or prevention of a disease associated with gene deregulation, overexpression, or mutation. For example, a nanostructure for replication and subgenomic expression may be introduced into a cancerous cell or tumor and thereby inhibit the expression of a gene required for or associated with maintenance of the carcinogenic/tumorigenic phenotype. To prevent a disease or other pathology, a target gene may be selected that is, e.g., required for initiation or maintenance of a disease/pathology. Treatment may include amelioration of any symptom associated with the disease or clinical indication associated with the pathology.

In addition, nanostructures for replication and subgenomic expression of the present invention are used to treat diseases or disorders associated with gene mutation. In one embodiment, a nanostructure for replication and subgenomic expression is used to modulate the expression of a mutated gene or allele. In such embodiments, the mutated gene is the target of the nanostructure for replication and subgenomic expression, which will comprise a region complementary to a region of the mutated gene. This region may include the mutation, but it is not required, as another region of the gene may also be targeted, resulting in decreased expression of the mutant gene or mRNA. In certain embodiments, this region comprises the mutation, and, in related embodiments, the resulting nanostructures for replication and subgenomic expression specifically inhibits expression of the mutant mRNA or gene but not the wildtype mRNA or gene. Such a nanostructure for replication and subgenomic expression is particularly useful in situations, e.g., where one allele is mutated but another is not. However, in other embodiments, this sequence would not necessarily comprise the mutation and may, therefore, comprise only a wild-type sequence. Such a nanostructure for replication and subgenomic expression is particularly useful in situations, e.g., where all alleles are mutated. A variety of diseases and disorders are known in the art to be associated with or caused by gene mutation, and the invention encompasses the treatment of any such disease or disorder with a nanostructure for replication and subgenomic expression.

In certain embodiments, a gene of a pathogen is targeted for inhibition. For example, the gene could cause immunosuppression of the host directly or be essential for replication of the pathogen, transmission of the pathogen, or maintenance of the infection. In addition, the target gene may be a pathogen gene or host gene responsible for the entry of a pathogen into its host, drug metabolism by the pathogen or host, replication or integration of the pathogen's genome, establishment or spread of an infection in the host, or assembly of the next generation of the pathogen. Methods of prophylaxis (i.e., prevention or decreased risk of infection), as well as reduction in the frequency or severity of symptoms associated with infection are included in the present invention. For example, cells at risk for infection by a pathogen or already infected cells, particularly human immunodeficiency virus (HIV) infections, may be targeted for treatment by the introduction of a nanostructure for replication and subgenomic expression according to the invention.

In other specific embodiments, the present invention is used for the treatment or development of treatments for cancers of any type. Examples of tumors that can be treated using the methods described herein include, but are not limited to, neuroblastomas, myelomas, prostate cancers, small cell lung cancer, colon cancer, ovarian cancer, non-small cell lung cancer, brain tumors, breast cancer, leukemias, lymphomas, and others.

The nanostructures for replication and subgenomic expression and expression vectors (including viral vectors and viruses) may be introduced into cells in vitro or ex vivo and then subsequently placed into an animal to affect therapy, or they may be directly introduced to a patient by in vivo administration. Thus, the invention provides methods of gene or antiviral therapy, in certain embodiments. Compositions of the invention may be administered to a patient in any of a number of ways, including parenteral, intravenous, systemic, local, oral, intratumoral, intramuscular, subcutaneous, intraperitoneal, inhalation, or any such method of delivery. In one embodiment, the compositions are administered parenterally, i.e., intraarticularly, intravenously, intraperitoneally, subcutaneously, or intramuscularly. In a specific embodiment, the liposomal compositions are administered by intravenous infusion or intraperitoneally by a bolus injection.

Regardless of the route of administration selected, the RNA nanostructure templates of the present invention, which may be used in a suitable hydrated form by conventional methods known to those of skill in the art or the compositions of the present invention which create virion-like nanoparticles that are composed of an RNA nanostructure core formulated into protein-encapsidated or protein-enveloped non-infectious virion-like nanoparticles using methods described in this application.

In preferred embodiments, the RNA nanostructures of this invention are virion-like nanoparticles composed of non-infectious RNA sequences (FIG. 4i) and encapsidated by protein (FIG. 4iii).

In other preferred embodiments, the RNA nanostructures of this invention are virion-like nanoparticles composed of non-infectious RNA sequences (FIG. 4i) and encapsidated by protein (FIG. 4iii), and further enveloped by one or more lipid proteins (FIG. 1v, v).

In still other preferred embodiments, the encapsidating proteins are viral structural proteins and such proteins are not expressed by the RNA nanostructure template (FIG. 13).

In yet other preferred embodiments, the encapsidating proteins are non-viral proteins and such proteins are expressed by the RNA nanostructure template.

Actual therapeutically effective dosage levels of the active RNA nanostructure templates or virion-like nanoparticles of this invention may be varied so as to obtain an amount of the active ingredient which is effective to achieve the desired therapeutic response for a particular patient, composition, and mode of administration.

The selected dosage level will depend upon a variety of factors including the activity of the particular compound of the present invention employed, or the ester, salt or amide thereof, the route of administration, the time of administration, the rate of excretion, the rate of viral infection, or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of this invention at levels lower than that required in order to achieve the desired therapeutic effect and gradually increase the dosage until the desired effect is achieved.

In general, a therapeutically effective dose this invention will be that amount which is the lowest dose effective to produce a therapeutic effect. Such an effective dose will generally depend upon the factors described above. Generally, oral, intravenous, intracerebroventricular, subcutaneous, or respiratory doses of the compounds of this invention for a patient, when used for the indicated analgesic effects, will range from about 0.0001 to about 1 μg per kilogram of body weight per dose when treating active viral infections or an upper range of 10 mg per kilogram of body weight when treating inactive viral infections or non-infectious disease. The preferred rate of dosing to treat an active viral infection is a single administration, but the treatment of non-infectious diseases with this invention may require daily, weekly, monthly or yearly dosing to achieve a therapeutic effect.

Compositions of the invention may be formulated as pharmaceutical compositions suitable for delivery to a subject. The pharmaceutical compositions of the invention will often further comprise one or more buffers (e.g., neutral buffered saline or phosphate buffered saline), carbohydrates (e.g., glucose, mannose, sucrose, dextrose, or dextran's), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. Alternatively, compositions of the present invention may be formulated as a lyophilizate.

The amount of nanostructures for replication and subgenomic expression administered to a patient can be readily determined by a physician based upon a variety of factors, including, e.g., the disease and the level of nanostructures for replication and subgenomic expression expressed from the vector being used (in cases where a vector is administered). The amount administered per dose is typically selected to be above the minimal therapeutic dose but below a toxic dose. The choice of the amount per dose will depend on a number of factors, such as the medical history of the patient, the use of other therapies, and the nature of the disease. In addition, the amount administered may be adjusted throughout treatment, depending on the patient's response to treatment and the presence or severity of any treatment-associated side effects.

The invention further includes a method of identifying gene function in an organism comprising the use of a nanostructure for replication and subgenomic expression to inhibit the activity of a target gene of previously unknown function. Instead of the time consuming and laborious isolation of mutants by traditional genetic screening, functional genomics envisions determining the function of uncharacterized genes by employing the invention to reduce the amount and/or alter the timing of target gene activity. The invention may be used in determining potential targets for pharmaceutics, understanding normal and pathological events associated with development, determining signaling pathways responsible for postnatal development/aging, and the like. The increasing speed of acquiring nucleotide sequence information from genomic and expressed gene sources, including total sequences for the yeast, D. melanogaster, and C. elegans genomes, can be coupled with the invention to determine gene function in an organism (e.g., nematode). The preference of different organisms to use particular codons, searching sequence databases for related gene products, correlating the linkage map of genetic traits with the physical map from which the nucleotide sequences are derived, and artificial intelligence methods may be used to define putative open reading frames from the nucleotide sequences acquired in such sequencing projects.

In one embodiment, a subviral nanoparticle is used to inhibit gene expression based upon a partial sequence available from an expressed sequence tag (EST), e.g., in order to determine the gene's function or biological activity. Functional alterations in growth, development, metabolism, disease resistance, or other biological processes would be indicative of the normal role of the EST's gene product.

The ease with which a nanostructure for replication and subgenomic expression can be introduced into an intact cell/organism containing the target gene allows the present invention to be used in high throughput screening (HTS). For example, solutions containing nanostructure for replication and subgenomic expression that are capable of inhibiting different expressed genes can be placed into individual wells positioned on a microtiter plate as an ordered array, and intact cells/organisms in each well can be assayed for any changes or modifications in behavior or development due to inhibition of target gene activity. The function of the target gene can be assayed from the effects it has on the cell/organism when gene activity is inhibited. In one embodiment, nanostructures for replication and subgenomic expression of the invention are used for chemogenomic screening, i.e., testing compounds for their ability to reverse a disease modeled by the reduction of gene expression using a nanostructure for replication and subgenomic expression of the invention.

If a characteristic of an organism is determined to be genetically linked to a polymorphism through RFLP or QTL analysis, the present invention can be used to gain insight regarding whether that genetic polymorphism may be directly responsible for the characteristic. For example, a fragment defining the genetic polymorphism or sequences in the vicinity of such a genetic polymorphism can be amplified to produce an RNA, a nanostructure for replication and subgenomic expression can be introduced to the organism, and whether an alteration in the characteristic is correlated with inhibition can be determined.

The present invention is also useful in allowing the inhibition of essential genes. Such genes may be required for cell or organism viability at only particular stages of development or cellular compartments. The functional equivalent of conditional mutations may be produced by inhibiting activity of the target gene when or where it is not required for viability. The invention allows addition of a nanostructure for replication and subgenomic expression at specific times of development and locations in the organism without introducing permanent mutations into the target genome. Similarly, the invention contemplates the use of inducible or conditional vectors that express a nanostructure for replication and subgenomic expression only when desired.

The present invention also relates to a method of validating whether a gene product is a target for drug discovery or development. A nanostructure for replication and subgenomic expression that targets the mRNA that corresponds to the gene for degradation is introduced into a cell or organism. The cell or organism is maintained under conditions in which degradation of the mRNA occurs, resulting in decreased expression of the gene. Whether decreased expression of the gene has an effect on the cell or organism is determined. If decreased expression of the gene has an effect, then the gene product is a target for drug discovery or development.

Steps for Designing Subviral RNAi Nanostructures

Step I—for Viral RdRp Based Replication and Subgenomic Transcription, Extract Sequences from a Modeled RNA Virus:
1. Define and note 5′ UTR (or 5′ UTR plus additional sequences as a 5′ leader) sequence including sequences up to and including the CS (conserved sequence) of the first TRS (LEADER TRS), intergenic region (IGR), cyclization sequence or transgenic fragment and up to the first coding gene. In some cases, some sequences within the first coding gene may be required. In the case of SARS-2, the 5′ UTR of interest is nucleotides 1-302 as viewable when the genome is pasted into a cloning application like SnapGene Viewer. Additionally, references to this and other coronavirus viral 5′ sequences can also be identified from published research (YANG D., 2015). Transcriptomic information can also be identified from published research (Dongwan et al., 2020) which defines all the functional secondary structures preferable in the SARS-2 5′ UTR and includes some sequences of nsp1 in order to complete the conserved secondary structure. (FIG. 8viii).
2. Define and note the 3′ UTR sequence. Note: Use this same 3′ sequence as the 3′ end of nanostructure if replication of the RNA nanostructure template is sought (see FIG. 1, 2). In the case of SARS-2, the 3′ UTR of interest is within nucleotides 29,675-29,903 (229 nt) of the published Wuhan SARS-2 nCov genome (SEQ ID NO: 3). Transcriptomic information can also be identified from published research (Dongwan et al., 2020).
3. Define and note the Leader-TRS, Body-TRS (transcription regulatory sequence), or intergenic regions for the model virus. In coronavirus, the TRS contains the CS of “ACGAAC” (Yount, 2006) with an additional dinucleotide variable region. Such TRS sequences are typical in virus that have subviral expression. In virus without subviral expression, a TRS is not typical. The CS region is different for different host virus. Note: If recombination is to be avoided, use an engineered TRS sequence or engineered intergenic region in place of both the Leader and Body-TRS sequences and avoid endogenous TRS sequences. The examples in this application utilize “AAUGGUCGC” as published by (Graham, 2018) as a way to keep attenuated-live coronavirus from recombination with wild type coronavirus during replication strand transfer when both templates are present. (Example, SEQ ID NO: 5)
4. Define an empty template by assemble 5′ Leader, Leader-TRS, copies of Body-TRS and 3′ UTR as needed and in a manner that resembles FIG. 7 i-iv, FIG. 8, FIG. 11Aii, FIG. 11Aiii. Then, design your “gene modulating” transcript sequences to be inserted in-between (FIG. 1), or incorporated with (FIG. 2, 7, 8), the Body-TRS sequences. Increase the number depending upon the number of subgenomic transcripts needed. Such transcripts can be coding or non-coding but are preferably non-coding gene modulating single-stranded RNA.

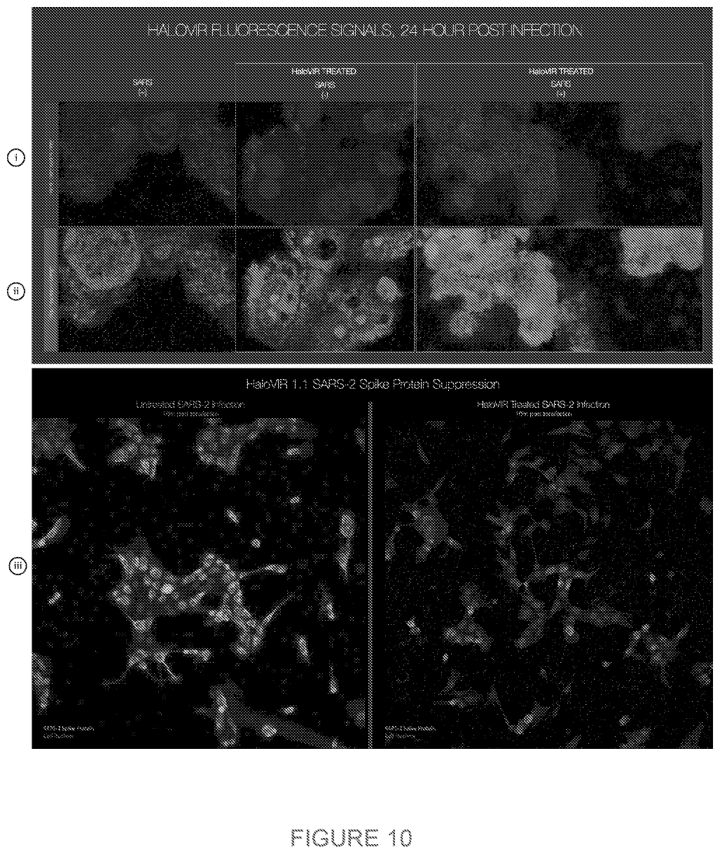

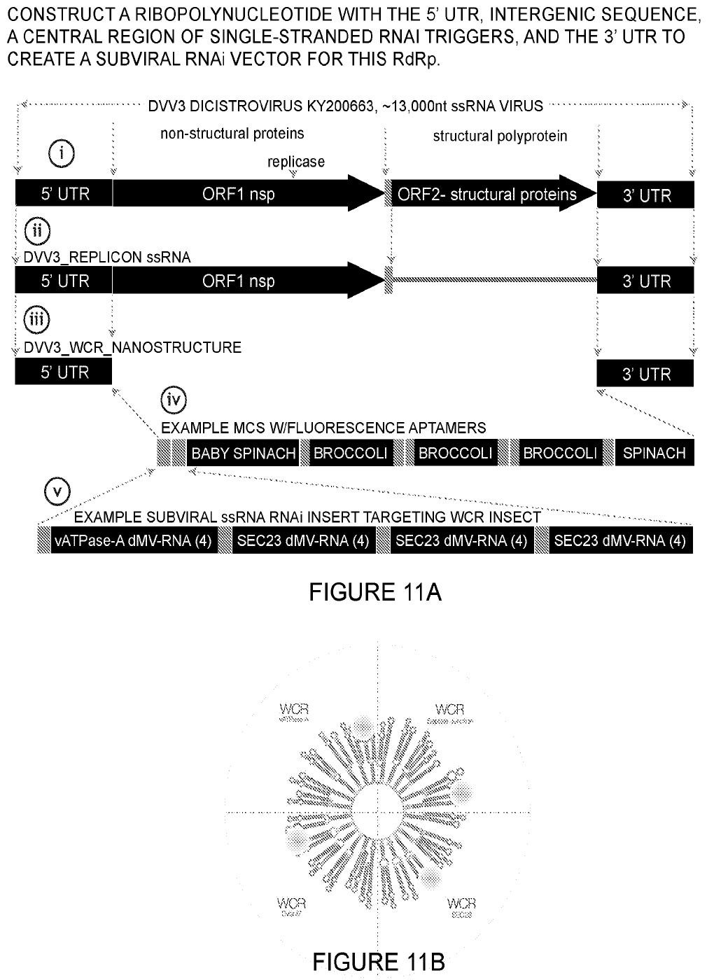

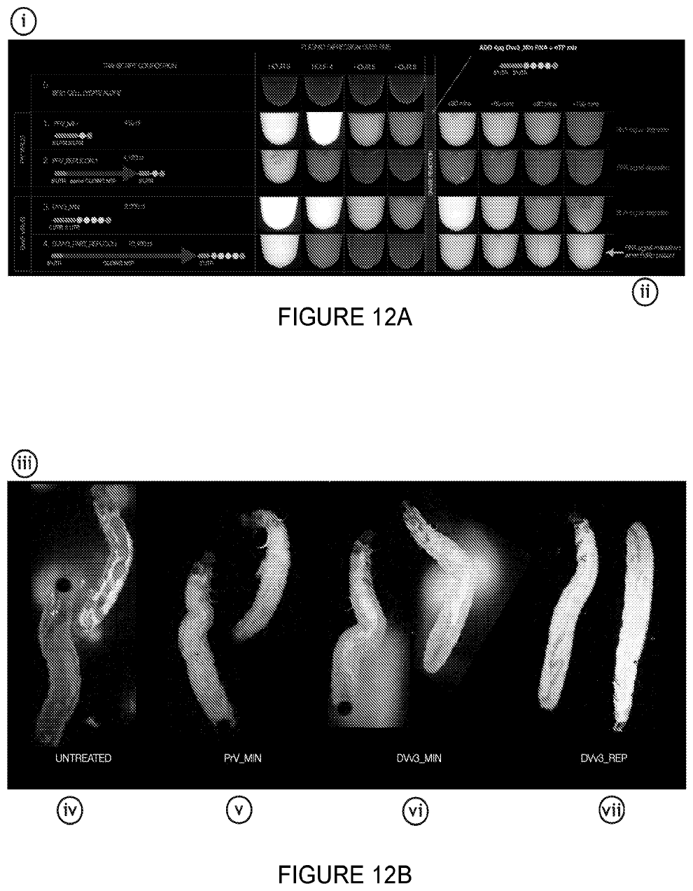

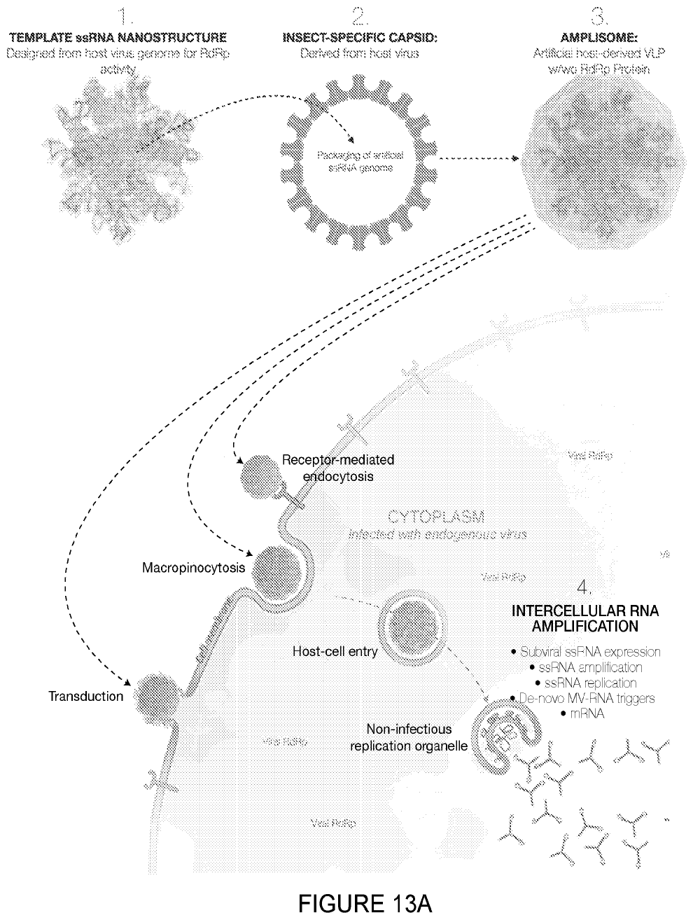

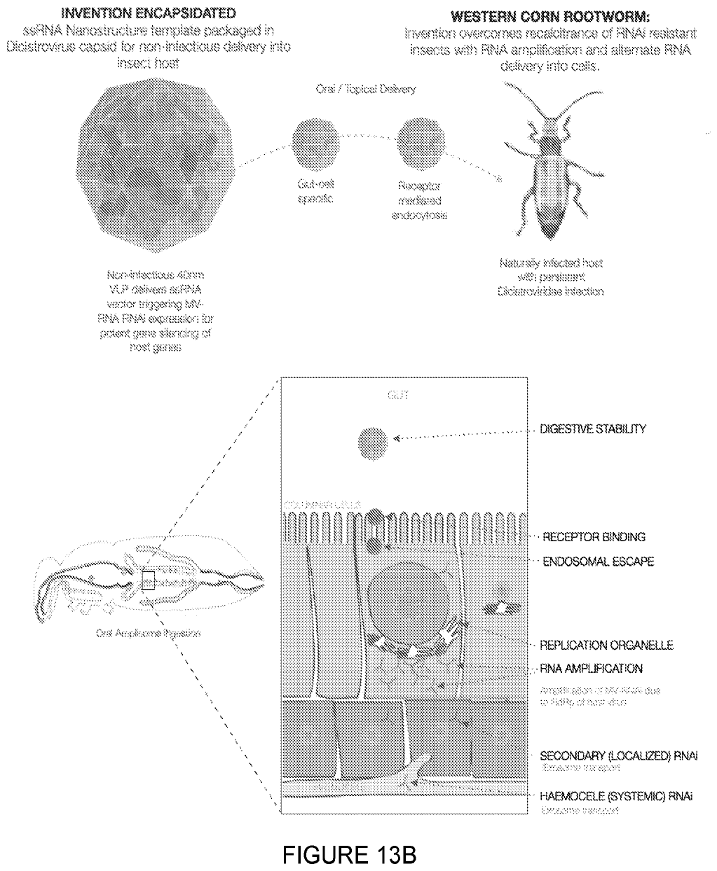

Step II—Design RNAi Triggers and Subgenomic Transcripts
5. Design and screen efficacious MV-RNA according to methods in U.S. Pat. No. 9,200,276. Use ′RNAi Cloud′ software to design all MV-RNA candidates for any number of gene targets. (Example, SEQ ID NO: 7-15)
6. It is preferred that a minimum of 6 different MV-RNA triggers are used in the nanostructure template to increase RNAi sites up to 12 when targeting viral genes.
7. Form group(s) MV-RNA or hairpin sequences. (Example, SEQ ID NO: 33, 41)
8. If the RNA nanostructure template is to elicit subgenomic transcription, group RNAi triggers like MV-RNA to form a subgenomic transcript (sgTranscript, FIG. 2) with at least dinucleotides in-between each MV-RNA or shRNA. Multiple sets of these can be designed in any number of orientations. For example, a general motif pattern for a group of MV-RNA might be:
   <MV-RNA>< . . . ><MV-RNA>< . . . ><MV-RNA>
   To elicit subgenomic expression, add Body-TRS sequences to your RNA group set. In certain embodiments, each group is flanked at the 5′ or 3′, or both 5′ and 3′ with a Body-TRS. For example, the motif pattern for a sgTranscript might be:
   <B-TRS><MV-RNA>< . . . ><MV-RNA>< . . . ><MV-RNA>
   In other embodiments, the Body-TRS is part of the group secondary structure (FIG. 9Ai, FIG. 9Biii). For example, the motif pattern for an sgTranscript might be:
   <<B-TRS>MV-RNA>< . . . ><MV-RNA>< . . . ><MV-RNA><B-TRS><MVRNA>< . . . ><MV-RNA>< . . . ><MV-RNA<B-TRS>>
   NOTE: Body-TRS sequences can be placed in-between individual or groups of RNAi triggers (subviral transcripts, i.e., FIG. 2Ai, 2Aii) as needed. See FIG. 1 as an example showing Body-TRS sequences in-between every 3rd MVRNA with each subgenomic transcript containing three MV-RNA in the configuration example of FIG. 1. Alternatively, Body-TRS sequences and structures can be combined with the group of MV-RNA's to be transcribed by subgenomic expression. The coronavirus example provides an example of TRS-B in-between MV-RNA groups (Example, SEQ ID NO: 20, nucleotides 226 . . . 400) and also with TRS-Body structure embedded in within the secondary structure of either the 5′ or the 3′ of the MV-RNA group transcript (FIG. 9). (Example, SEQ ID NO: 20, nucleotides 722 . . . 960)

Step III—Assemble Nanostructure Templates Based on Subgenomic Modality:

In order to determine the replication and/or subviral expression modality of the nanostructure templates of this invention when modeled for a given single-stranded RNA virus, one appends the central RNA region (designed above) with either a 5′ leader, and/or a 3′ UTR of the modeled viral genome. For example and without limitation, candidate RNA nanostructure compositions to determine minimum motifs for a modeled virus and for activity of the associated RdRp activity are;

### A. <5′ UTR LEADER>+<central RNA>

B. <central RNA>+<3′ UTR>

### C. <5′ UTR LEADER>+<central RNA>+<3 ′UTR>

Without limitation, it is expected that for most viral genomes being applied in the methods of this invention that a minimum 5′ and/or 3′ composition is required for the synthesis of at least a partial (−) strand by the RdRp of the same virus and that the (−) strand forms at least a partial dsRNA with the RNA nanostructure template. One skilled in the art can determine whether the virus being modeled and the related RdRp requires the 5′ leader, 3′ UTR, or both for strand synthesis by detecting the presence of dsRNA intermediates. Example dsRNA detection methods are anti-dsRNA antibodies, dsRNA dyes, PCR, or other standard electrophoresis methods following exposure to the RdRp(s) of interest. Without limitation, the exposure the RdRp to determine minimum RNA compositions can be done by co-expression of the RdRp mRNA from the 5′ leader of each candidate RNA nanostructure composition, or transfection of the candidate RNA nanostructure composition RNA's into cells infected with the virus being modeled, or co-transfection of the candidate RNA nanostructure RNA and RdRp protein into cells, or co-incubation of the candidate RNA nanostructure RNA and RdRp protein in in-vitro transcription reactions.

Such minimum compositions and resulting dsRNA may not lead to de-novo synthesis “replication” of the single-stranded RNA nanostructure template but forms the basis for adding either “replication” and/or is a minimum requirement for “subgenomic expression” activity from the RNA nanostructure template. From the minimum composition, one can determine whether or not to add additional features like replication of the RNA template nanostructure, or subviral expression from the RNA nanostructure template as a vector if a modeled RNA virus has subviral RNA features. The examples demonstrate a virus with many subviral promoters (SARS-2 coronavirus), a single subviral promoter (Dicistrovirus DVVv3), and one that is without subviral transcription and relies on replication (Dengue virus). One can chose modalities based on a modeled RNA virus;

A) Non-replicating+subgenomic expression
B) Replicating+subgenomic expression
C) Replicating w/o subgenomic expression.

Synthesis of an Intermediary Nanostructure Strand

The composition to achieve at least a single synthesis event of an intermediary RNA—which is a reverse-complement of some or all of the nanostructure template. In most cases, the synthesis of an intermediary strand requires a motif at the 5′ or the 3′ end of the nanostructure template.

In preferred embodiments, the RNA nanostructure templates of this invention leads to the synthesis of a complementary intermediary strand due to the activity of RdRp or an RdRp-like protein. In such cases, the 5′ UTR or the 3′ UTR end is capable of promoting polymerase activity.

In other preferred embodiments, the RNA nanostructure templates of this invention on the 5′ end or 3′ end is composed of viral 5′ UTR or 3′ UTR sequences.

In other preferred embodiments, the RNA nanostructure templates of this invention on the 5′ end or 3′ end is composed of viral sequences that are derived beyond the borders of the 5′ UTR or 3′ UTR (FIG. 8ii, 8vii-x, SEQ ID NO: 2, 20).

In still other preferred embodiments, the RNA nanostructure templates of this invention on 3′ end is composed of QB Replicase promoting sequences.

In this mode, only the synthesis of a reverse-complement to all or part of the nanostructure template is enabled. This is useful for non-replicative uses of this invention.
1. Assemble the sequences in STEP I & II to elicit synthesis of intermediary strand:
   <5′UTR>< . . . At least one group from STEP I, II . . . >
or
< . . . At least one group from STEP I, II . . . ><3′UTR>
or
< . . . At least one group from STEP I, II . . . ><QB Replicase seq.>

Mode A: Non-Replicating+Subgenomic Transcription

The composition for subgenomic expression typically requires first the single synthesis event of an intermediary RNA (above)—which is a reverse complement of some or all of the nanostructure template.

In most cases, the synthesis of an intermediary strand and subgenomic transcription requires a motif at the 5′ or the 3′ end of the nanostructure template, a Leader-TRS, and a Body-TRS or subgenomic promoter flanking the internal subgenomic transcription region (SEE DRAWINGS).

In preferred embodiments, the RNA nanostructure templates of this invention replicates the template by first synthesizing a complementary intermediate strand due to activity of RdRp or RdRp-like protein and continues to use this intermediate strand as a template for de novo synthesis of a new nanostructure template sequence. In such cases, the 5′ UTR end or the 3′ UTR end is capable of promoting polymerase activity, and the 5′ UTR end or 3′ UTR end is further capable of promoting polymerase activity.

In specific embodiments, the RNA nanostructure templates of this invention on the 5′ end and the 3′ end is composed of viral 5′ UTR or 3′ UTR sequences.

In other specific embodiments, the RNA nanostructure templates of this invention on the 5′ end and the 3′ end is composed of viral 5′ UTR or 3′ UTR sequences that are mutated.

In this mode, continuous synthesis and replication of the RNA nanostructure template are enabled. This is useful for replicative uses of this invention.
1. Assemble the sequences in STEP I & II to intermediary strand synthesis+subgenomic expression:
   <5′UTR or leader><L-TRS>< . . . At least one group from STEP I, II . . . ><3′UTR>
   or
   <5′UTR or leader><L-TRS><B-TRS>< . . . At least one group from STEP I, II . . . ><B-TRS>
   or
   <5′UTR or leader><L-TRS><B-TRS>< . . . At least one group from STEP I, II . . . ><B-TRS><3′UTR>

Mode B: Replicating+Subgenomic Transcription

The composition for subgenomic expression typically requires first the single synthesis event of an intermediary RNA (above)—which is a reverse complement of some or all of the nanostructure template.

In most cases, the synthesis of an intermediary strand and subgenomic transcription requires a motif at the 5′ or the 3′ end of the nanostructure template or both. For subviral activity, a Leader-TRS, and a Body-TRS or subgenomic promoter flanking the internal subgenomic transcription region (SEE DRAWINGS).

In preferred embodiments, the RNA nanostructure templates of this invention replicates the template by first synthesizing a complementary intermediate strand due to activity of RdRp or RdRp-like protein and continues to use this intermediate strand as a template for de novo synthesis of a new nanostructure template sequences in a continuous transcription cycle. In such cases, both the 5′ UTR end or the 3′ UTR end are capable of promoting and supporting polymerase activity.

In preferred embodiments, the 5′ end and the 3′ end of the RNA nanostructure templates of this invention are composed of virally derived 5′ UTR that contains a leader-TRS, at least one internal motif from STEP I-II flanked by at least one Body-TRS, and virally derived 3′ UTR sequences. The TRS or subgenomic promoters can be endogenous, engineered, or derived from virus untypical to the host.

In this mode, continuous synthesis and replication of the RNA nanostructure template is enabled and the presence of a Leader-TRS and a Body-TRS (or equivalent) lead to subgenomic transcription of at least part of the RNA flanking the Body-TRS. This is useful for replicative uses of this invention where additional transcription of gene modulating single-stranded RNA is preferred.
1. Assemble the sequences in STEP I & II to elicit replication+subgenomic expression:
   <5′UTR, L-TRS><B-TRS>< . . . At least one group from STEP I, II . . . ><BTRS><3′UTR>
   or
   <5′UTR, L-TRS>< . . . At least one group from STEP I, II . . . ><BTRS><3′UTR>
   or
   <5′UTR, L-TRS><B-TRS>< . . . At least one group from STEP I, II . . . ><3′UTR>

Mode C: Replication W/O Subgenomic Transcription

In preferred embodiments, the RNA nanostructure templates of this invention replicates the template by first synthesizing a complementary intermediate strand due to activity of RdRp or RdRp-like protein and continues to use this intermediate strand as a template for de novo synthesis of a new nanostructure template sequence in a continuous transcription cycle. In such cases, the 5 ′UTR end or the 3 ′UTR end is capable of promoting and supporting polymerase activity.

In specific embodiments, the 5′ end and the 3′ end of the RNA nanostructure templates of this invention are composed of viral 5′ UTR or 3′ UTR sequences.

In this mode, continuous synthesis and replication of the RNA nanostructure template is enabled. This is useful for replicative uses of this invention.
1. Assemble the sequences in STEP I & II to elicit replication:
   <5′UTR or leader>< . . . At least one group from STEP I, II . . . ><3′UTR>

Optional Step III—Fluorogenic Aptamers for Subviral Detection

Fluorescent RNA aptamers can be used qualitative and quantitative measurement of both replication and/or subgenomic transcription of the nanostructure templates of this invention. In either case, a specific fluorescent aptamer is place 3′ of the subgenomic promoter on the sgTranscript, and/or on the 3′ end of the 5′ UTR of this invention to track replication. Sequences of interest are common in the art. A couple examples are provided:

Mango (Dolgosheina E.V., et al., 2014),,
SEQ ID NO: 18
CGAAGGAGAGGAGAGGAAGAGGAGAG
Broccoli (Filonov et al., 2014), USPTO# 61/
874,819,,
SEQ ID NO: 17
GGAGACGGUCGGGUCCAGAUAUUCGUAUCUGUCGAGUAGAGUGUGGGCU
CC

In certain embodiments, the fluorogenic aptamer is in reverse-complement orientation within the RNA nanostructure template and is use to measure strand synthesis of intermediary strands or subgenomic expression of (−) oriented transcripts.

Step IV—Aptamers for Encapsidation and/or Packaging

ShRNA hairpins can be used in place of a portion of the MV-RNA, but MV-RNA or 3-way junction containing constructs are ideal to accommodate the plurality of moiety-binding aptamers that are preferred for binding proteins leading to intracellular encapsidation and/or packaging of the RNA nanostructure template into subvirions (see FIG. 4, 6). In the coronavirus example provided, MV-RNAs are utilized and aptamer binds Coronavirus Nucleocapsid to elicit intracellular encapsidation when in infected cells, as discussed in this application. Design nanostructure cores according to PCT/US16/48492 using results from STEP II. Replace the loop regions of the MV-RNA with the aptamer sequence designed to bind to the encapsidation protein of choice. The provided example is an aptamer that binding to the N protein of Coronavirus (SEQ ID NO: 20 nucleotides 1030 . . . 1117).

Step V: Insert Subgenomic Transcripts into Nanostructure Template

Create one or more sequences made up of groups of gene modulating single-stranded RNA (i.e., MV-RNA), but each group made of at least one RNAi sequence or aptamer, and each group sequence with or without flanking subgenomic promoter(s)—depending upon the desired modality explained above.

In general, a group will be a collection of MV-RNA RNAi triggers separated by dinucleotides UU, UC, or other pairs. However, other RNA can be used such as shRNA, pre-miRNA, pRNA, etc. Example:
<MV-RNA 1>n . . . n<MV-RNA 2>n . . . n<MV-RNA 3>n . . . n<MV-RNA 4>

In Step IV above, one or more of the example MV-RNA within the group may contain aptamer(s) for encapsidation or fluorogenic detection, etc., or one or more of these examples MV-RNA could be flanked by an aptamer or subgenomic promoter secondary structure to support a subgenomic expression modality of this sequence. Example:
sgTranscript=<subgenomic promoter stem-loop>n . . . n<MV-RNA 1>n . . . n<MV-RNA 2>n . . . n<MV-RNA 3>n . . . n<MV-RNA 4>nn<fluorogenic aptamer>n . . . n<subgenomic promoter stem-loop>

In general, at least one group containing RNA of interest, like MV-RNA of a desired plurality, aptamers, promoters, enhancers, etc. is designed then inserted in-between the 5′ and 3′ UTR sequences appropriately determined in STEP I. Example, a 5′ UTR, engineered TRS, a subgenomic transcript, engineered TRS, 3′ UTR:
<5′ leader w/eTRS><optional coding CDS><eTRS>< . . . subgenomic transcript “sgTranscript”.>><eTRS>< . . . subgenomic transcript “sgTranscript”><3′ UTR>

As an example constructs using this invention, RNA nanostructure templates with multiple modalities are provided as an antiviral targeting the coronavirus of a host (SEQ ID NO: 20) and two examples of targeting a host of a virus; the Dengue virus infection-induced RNA nanostructure targeting infected Aedes mosquitos (SEQ ID NO: 34) and the Western Corn Rootworm crop protection biopesticide with either virus infection-induced RNA nanostructure RNAi amplification (SEQ ID NO: 44) or non-infectious self-amplifying RNA nanostructures a modeled RNA virus endogenous of the host (SEQ ID NO: 39).

Step V. Verifying Nanoparticle Folding with Final Aptamer(s):

Once the full sequence of each MV-RNA or MV-RNA group subgenomic transcript is designed and inserted into the nanostructure template containing the features for the desired modality, confirm trigger-to-aptamer/intramer orientation sequences within the nanostructure to be that similar to overall FIG. 1, subgenomic FIG. 3 with or without the alternative secondary structures of FIG. 9. Computationally fold the RNA in a computer program like cofold, or specifically, Multivalent RNAi Cloud computationally verifies the integrity of the secondary structure of the final nanostructure and its parts. In general, the 5′ and 3′ UTR will not interfere with the folding of the gene modulating regions, but care must be taken to confirm that long-distance interactions will not cause unexpected folding results. Folding software based on thermodynamics may not provide as accurate of results as cofold-transcription-based folding.

The resulting fold notation or art will indicate free nucleotides as “.” and bound nucleotides as “(” or “)”. Relative Free-energy and melting temperature will also give an indication as to the stability of the precise transcript. One can view the resulting art representing the precisely structured transcript.

Example Fold Notation:

These methods, in certain embodiments, include determining or predicting the secondary structure adopted by the sequences selected in STEP (II), e.g., in order to determine that they are capable of adopting a stem-loop or 3-way junction structure.

Similarly, these methods can include a verification step, which comprises testing the designed RNA sequence for its ability to inhibit the expression of a target gene, e.g., in an in vivo or in vitro test system.

The invention further contemplates the use of a computer program to select MV-RNA sequences of the nanostructure, based upon the complementarity characteristics described herein. The invention, thus, provides computer software programs, and computer-readable media comprising said software programs to be used to select the RNA nanostructure sequences, as well as computers containing one of the programs of the present invention.

In certain embodiments, a user provides a computer with information regarding the sequences, locations, or names of the target gene(s). The computer uses this input in a program of the present invention to identify one or more appropriate regions of the target gene to target in MV-RNA formats and outputs or provides complementary sequences to use with the 5′ Leader, 3′ UTR, and multiple TRS sequences for the assembly of the RNA nanostructure of the invention. Typically, the program will select a series of sequences that are not complementary to a genomic sequence, including the target gene or the region of the RNA nanoparticle that is complementary to the target gene. When desired, the program also provides sequences of gap regions, fold notations, and fold art. Upon selecting appropriate MV-RNA orientations, plurality, aptamers, loops, linkages, TRS, Leader, 3′ UTR, Opening/Closing sequence, cloning sites, and necessary TRS transcription elements, the computer program outputs or provides this information to the user.

The programs of the present invention may further use input regarding the genomic sequence of the organism containing the target gene, e.g., public or private databases, as well as additional programs that predict the secondary structure and/or hybridization characteristics of particular sequences, to ensure that the RNA nanoparticle adopts the correct secondary structure (i.e., mFold, RNAfold, cofold, RNAi Cloud) and does not hybridize to non-target genes (BLASTn).

Step VII. Nanostructure RNA Production

Once the preferred 5′ Cap composition, optional 5′ UTR, Leader-TRS, each transcript with Body TRS sequences in-between (FIG. 1) and/or embedded (FIG. 9Ai, 9Biii), and an optional 3′ UTR is assembled as a single sequence, it is ready to be incorporated into the appropriate transcriptional setting. One skilled in the art will know how to select the appropriate transcription promoter and termination motif. If transcripts are produced in-vitro for human use, special attention should be placed on appropriate 5′ capping (i.e. CleanCap 1) and base methylation during transcription to avoid immune stimulation in humans.

The coronavirus example (FIG. 1I, SEQ ID NO: 20) was produced by in-vitro T7 expression. Nucleocapsid protein was produced by recombinant expression in E. coli BL21 or Sf21 cells, then HIS purified.

The in vitro transcribed RNA nanostructure template at a concentration of 4 μM and the purified recombinant protein, in this case nucleocapsid at a 4× molar excess over RNA, were mixed in a recombination buffer (50 mM HEPES pH 7.5, 3 mM CaCl2), 150 mM NaCl) in an overnight dialysis at 4 C with a 3 kDa cutoff to encapsidate the RNA nanostructure with the nucleocapsid protein.

The resulting Core RNA with bound Nucleoprotein surface was then loaded into various lipid compositions for testing of the full lipid nanoparticle. For this coronavirus example, emphasis was placed on lipid compositions that would result in delivery into cells into a membranous location due to the many membrane-bound proteins of this virus needed for replication and subgenomic expression.

For encapsidation with non-enveloped capsid proteins, a lower pH is typically preferred for the formation of the procapsid, i.e., a recombination buffer (50 mM NaPO4 pH 6, 3 mM CaCl2), 150 mM NaCl)

Further in vitro validation of the RdRp-like activity of the RNA nanostructure template can be tested using recombinant RdRp protein and appropriate nucleotides and buffers to support in-vitro RdRp reactions.

The present invention is based, in part, upon the discovery that a highly-structured RNA nanostructure can simultaneously support subgenomic expression, shape-shifting, and replication by RdRp or RdRp-like proteins, and further enable intracellular packaging into membrane enveloped particles within cells, as described herein, and is extremely effective at expanding the utility of RNA nanostructures.

Additionally, this present invention is based, in part, upon the discovery that the sensitivity of MV-RNA RNAi triggers to loss of function by mutagenesis may enable subviral-based gene modulating biotechnology for commercial uses in medicine and agriculture.

Lastly, this present invention is based, in part, upon the combination of a structural RNAi trigger, compact nanostructure, and aptamer-driven nanoparticle formulation to the engineered encapsidation and packaging of the RNA nanostructures of this invention into subviral nanoparticles that typically would require endogenous packaging sequence(s) (Woo J et al., 2019, Masters P. et al., 2019). The resulting replication-competent, subgenomic expressing RNA nanoparticle compositions of this invention offer significant advantages over previously described techniques, including a first-of-its-kind method for combating viral infections of positive single-stranded virus, novel biopesticidal products, or in other embodiments shape-shifting from a spherical nanostructure into a dsRNA to elicit immune stimulation and gene silencing in cancerous cells.

The practice of the present invention will employ a variety of conventional techniques of cell biology, molecular biology, microbiology, and recombinant DNA, which are within the skill of the art. Such techniques are fully described in the literature. See, for example, Molecular Cloning: A Laboratory Manual, 2nd Ed., ed. by Sambrook, Fritsch, and Maniatis (Cold Spring Harbor Laboratory Press, 1989); and DNA Cloning, Volumes I and II (D. N.

### CITATIONS

1. Aaskov, J., K. Buzacott, H. M. Thu, K. Lowry, and E. C. Holmes. 2006. Longterm transmission of defective RNA viruses in humans and Aedes mosquitoes. Science 311:236-238
2. Ahn D G, Jeon I J, Kim J D, et al. RNA aptamer-based sensitive detection of SARS coronavirus nucleocapsid protein. Analyst. 2009; 134(9):1896-1901. doi:10.1039/b906788d
3. Barrett ADT, Dimmock N J. 1986. Defective interfering viruses and infections of animals. Curr. Top. Microbiol. Immunol. 128:55-84. doi:10.1007/978-3-642-71272-2_2
4. Binder S, Levitt A M, Sacks J J, Hughes J M. 1999. Emerging infectious diseases: Public health issues for the 21st century. Science 284(5418)
5. Carthew R W, Sontheimer E J. 2009. Origins and mechanisms of pre-miRNAs and siRNAs. Cell 136:642-655. doi:10.1016/j.cell.2009.01.035
6. Charon, J., Grigg, M. J., Eden, J. S., Piera, K. A., Rana, H., William, T., Rose, K., Davenport, M. P., Anstey, N. M., & Holmes, E. C. (2019). Novel RNA viruses associated with Plasmodium vivax in human malaria and Leucocytozoon parasites in avian disease. PLoS pathogens, 15(12)
7. Clyde K, Jennifer L. Kyle, Eva Harris, Recent Advances in Deciphering Viral and Host Determinants of Dengue Virus Replication and Pathogenesis, Journal of Virology November 2006, 80 (23) 11418-11431
8. Cui, Lei, 2015. The Nucleocapsid Protein of Coronaviruses Acts as a Viral Suppressor of RNA Silencing in Mammalian Cells. Journal of Virology August 2015, 89 (17) 9029-9043; DOI: 10.1128/JVI.01331-15
9. Cullen B R. 2009. Viral RNAs: lessons from the enemy. Cell 136:592-597. doi:10.1016/j.cell.2009.01.048
10. Domingo, E., and J. J. Holland. 1997. RNA virus mutations and fitness for survival. Annu. Rev. Microbiol. 51:151-178
11. Dimmock N J, Dove B K, Meng B, Scott P D, Taylor I, Cheung L, Hallis B, Marriott A C, Carroll M W, Easton A J. 2012. Comparison of the protection of ferrets against pandemic 2009 influenza A virus (H1N1) by 244 DI virus and oseltamivir. Antiviral Res. 96:376-385. doi:10.1016/j.antiviral.2012.09.017
12. Dolgosheina E V, Jeng S C, Panchapakesan S S, et al. RNA mango aptamer-fluorophore: a bright, high-affinity complex for RNA labeling and tracking. ACS Chemical Biology. 2014 October; 9(10):2412-2420. DOI: 10.1021/cb500499x.
13. Elfiky A A. SARS-CoV-2 RNA dependent RNA polymerase (RdRp) targeting: an in silico perspective [published online ahead of print, 2020 May 6]. J Biomol Struct Dyn. 2020; 1-9. doi:10.1080/07391102.2020.1761882
14. Ford R J., Amy M. Barker, Saskia E. Bakker, Robert H. Coutts, Neil A. Ranson, Simon E. V. Phillips, Amen R. Pearson, Peter G. Stockley, Sequence-Specific, RNA—Protein Interactions Overcome Electrostatic Barriers Preventing Assembly of Satellite Tobacco Necrosis Virus Coat Protein, Journal of Molecular Biology, Volume 425, Issue 6, 2013, Pages 1050-1064
15. Gao Y. Structure of the RNA-dependent RNA polymerase from COVID-19 virus. Science 2020: 779-782
16. Graham, R. L., Deming, D. J., Deming, M. E. et al. Evaluation of a recombination-resistant coronavirus as a broadly applicable, rapidly implementable vaccine platform. Commun Biol 1, 179 (2018).
17. Hentzschel F, Mitesser V, Fraschka S A, et al. Gene knockdown in malaria parasites via non-canonical RNAi. Nucleic Acids Res. 2020; 48(1):e2.
18. Hsieh P K, Chang S C, Huang C C, et al. Assembly of severe acute respiratory syndrome coronavirus RNA packaging signal into virus-like particles is nucleocapsid dependent. J Virol. 2005; 79(22):13848-13855. doi:10.1128/JVI.79.22.13848-13855.2005
19. Hurt A C, Chotpitayasunondh T, Cox N J, Daniels R, Fry A M, Gubareva L V, Hayden F G, Hui S D, Hungnes O, Lackenby A, Lim W, Meijer A, Penn C, Tashiro M, Uyeki T M, Zambon M, WHO Consultation on Pandemic Influenza A (H1N1) 2009 Virus Resistance to Antivirals. 2012. Antiviral resistance during the 2009 influenza A H1N1 pandemic: public health, laboratory, and clinical perspectives. Lancet Infect. Dis. 12:240-248. doi:10.1016/S1473-3099(11)70318-8
20. Jingyue Ju, Xiaoxu Li, Shiv Kumar, Steffen Jockusch, Minchen Chien, Chuanjuan Tao, Irina Morozova, Sergey Kalachikov, Robert N. Kirchdoerfer, James J. Russo, Nucleotide Analogues as Inhibitors of SARS-CoV Polymerase, bioRxiv 2020.03.12.989186
21. Jiwon Woo, Eunice Yoojin Lee, Mirae Lee, Taeyeon Kim, Yong-Eun Cho, An in vivo cell-based assay for investigating the specific interaction between the SARS-CoV N-protein and its viral RNA packaging sequence, Biochemical and Biophysical Research Communications, Volume 520, Issue 3, 2019, Pages 499-506
22. Jones K E, Patel N G, Levy M A, Storeygard A, Balk D, Gittleman J L, Daszak P. 2008. Global trends in emerging infectious diseases. Nature 451(7181)
23. Lai, M., RNA recombination in animal and plant viruses., March 1992, Microbiology and Molecular Biology Reviews
24. Lopez S B G, Guimarães-Ribeiro V, Rodriguez J V G, et al. RNAi-based bioinsecticide for Aedes mosquito control. Sci Rep. 2019; 9(1):4038.
25. Lynch M, Ackerman M S, Gout J F, Long H, Sung W, Thomas W K, Foster P L. 2016. Genetic drift, selection and the evolution of the mutation rate. Nat Rev Genet 17:704-714
26. Maida Y., 2009, An RNA-dependent RNA polymerase formed by TERT and the RMRP RNA, Nature, Vol 461, pages 230-237.
27. Maida Y, Yasukawa M, Masutomi K. De Novo RNA Synthesis by RNA-Dependent RNA Polymerase Activity of Telomerase Reverse Transcriptase. Mol Cell Biol. 2016; 36(8):1248-1259. Published 2016 Mar. 31. doi:10.1128/MCB.01021-15
28. Masters P S (2006) The molecular biology of coronaviruses. Adv Virus Res 66:193-292.
29. Masters P S (2019) Coronavirus genomic RNA packaging. Virology Vol. 537, pages 198-207.
30. Mendes A., Marli Vlok, James R Short, Tsutomu Matsui, Rosemary A Dorrington, An encapsidated viral protein and its role in RNA packaging by a non-enveloped animal RNA virus, 2015, Virology, Volume 476, Pages 323-333
31. Modrow S, Falke D, Truyen U, Schãtzl H (2013). “Viruses with Single-Stranded, Positive-Sense RNA Genomes” Molecular virology. Berlin, Heidelberg: Springer. pp. 185-349.
32. Morens D M, Folkers G K, Fauci A S. 2004. The challenge of emerging and re-emerging infectious diseases. Nature 430(6996)
33. Nagy P O, Simon A E (1997), New Insights Into the Mechanisms of RNA Recombination, Virology 235:1-9
34. Nigel J (2014). Defective Interfering Influenza Virus RNAs: Time To Reevaluate Their Clinical Potential as Broad-Spectrum Antivirals? Journal of Virology April 2014, 88 (10) 5217-5227
35. Reich, E., Franklin, R. M., Shatkin, A. J., and Tatum, E. L. (1961). Effect of actinomycin D on cellular nucleic acid synthesis and virus production. Science 134, 556-557. doi: 10.1126/science.134.3478.556
36. Sanjuan R, Domingo-Calap P. 2016. Mechanisms of viral mutation. Cell Mol Life Sci 73:4433-4448
37. Schneemann A., The structural and functional role of RNA in icosahedral virus assembly Annu. Rev, Microbiol., 60 (2006), pp. 51-67
38. Sijun Liu, Muting Chen, Thomas W. Sappington, Bryony C. Bonning, Genome Sequence of a Novel Positive-Sense, Single-Stranded RNA Virus Isolated from Western Corn Rootworm, Diabrotica virgifera virgifera. 10.1128/genomeA.00366-17
39. Sola I, Mateos-Gomez P A, Almazan F, Zuñiga S, Enjuanes L RNA-RNA and RNA-protein interactions in coronavirus replication and transcription. RNA Biol. 2011
40. Stockley, P. G., Ranson, N. A. & Twarock, R. (2013). A new paradigm for the roles of the genome in ssRNA viruses. Future Virol, in press.
41. Song W, Filonov G S, Kim H, et al. Imaging RNA polymerase III transcription using a photostable RNA-fluorophore complex. Nat Chem Biol. 2017; 13(11):1187-1194
42. Suttle C A (September 2005). “Viruses in the sea”. Nature. 437 (7057): 356-61.
43. Tautz N, Tews B A, Meyers C (2015) The molecular biology of pestiviruses. Adv Virus Res 93:47-160.
44. Tews B. A., Meyers G. (2017) Self-Replicating RNA. hi: Kramps T., Elbers K. (eds) RNA Vaccines. Methods in Molecular Biology, vol 1499. Humana Press, New York, N.Y.
45. Uchil P D, Satchidanandam V. Architecture of the flaviviral replication complex. Protease, nuclease, and detergents reveal encasement within double-layered membrane compartments. J Biol Chem. 2003; 278:24388-24398
46. Vasilakis N, Tesh R B (December 2015). “Insect-specific viruses and their potential impact on arbovirus transmission”. Current Opinion in Virology. 15: 69-74.
47. Warner, K. D., Chen, M. C., Song, W., Strack, R. L., Thorn, A., Jaffrey, S. R., & Ferré-D'Amaré, A. R. (2014). Structural basis for activity of highly efficient RNA mimics of green fluorescent protein. Nature structural & molecular biology, 21(8), 658-663
48. Woolhouse M E, Gowtage-Sequeria S. 2005. Host range and emerging and reemerging pathogens. Emerg Infect Dis 11(12)
49. Yamagata, Y., Muramoto, Y., Miyamoto, S., Shindo, K., Nakano, M., and Noda, T. (2019). Generation of a purely clonal defective interfering influenza virus. Microbiol. Immunol. 63, 164-171. doi: 10.1111/1348-0421.12681
50. Yang 0, Leibowitz J L. The structure and functions of coronavirus genomic 3′ and 5′ ends. Virus Research. 2015 August; 206:120-133. DOI: 10.1016d.virusres.2015.02.025.
51. Yount, B., Roberts, R. S., Lindesmith, L., Baric, R. S., et al. Rewiring the severe acute respiratory syndrome coronavirus (SARS-CoV) transcription circuit: Engineering a recombination-resistant genome. Proceedings of the National Academy of Sciences August 2006, 103 (33) 12546-12551.
52. Zamora H., Luce R., Biebricher C K., 1995, Design of artificial short-chained RNA species that are replicated by Q beta replicase. Biochemistry 34:1261-1266.

1. A single-stranded polyribonucleotide comprising:
(a) a 5′ leader sequence;
(b) a central RNA sequence, following the 3′ end of the leader sequence, comprising of one or more MV-RNA (multivalent-RNA) sequences; and
(c) a 3′ untranslated region (UTR) sequence following the central RNA sequence;
wherein the 5′ leader sequence, the 3′ UTR sequence, or both promote synthesis of a reverse-complement single-stranded polyribonucleotide transcript when combined with an RNA polymerase (RdRp) of an RNA virus or RdRp-like protein.

2. The single-stranded polyribonucleotide of claim 1, wherein the central RNA sequence further comprises one or more aptamer sequences.

3. The single-stranded polyribonucleotide of claim 1, wherein the central RNA sequence further comprises one or more short hairpin sequences.

4. The single-stranded polyribonucleotide of claim 1 further comprising:
an intergenic or transcription regulatory sequence (TRS) or cyclization sequence, following the 3′ end of the 5′ leader sequence and before the 5′ end of the central RNA sequence, to promote subviral expression.

5. The single-stranded polyribonucleotide of claim 4, wherein the intergenic or transcription regulatory sequence (TRS) followed by the central RNA sequence is duplicated at least one time after the 3′ end of the central RNA sequence and before the 5′ end of the 3′ UTR and the duplicated intergenic or transcription regulatory sequence (TRS) promotes subviral transcription of at least part of the duplicated central RNA sequence.

6. The single-stranded polyribonucleotide of claim 5, wherein each intergenic or transcription regulatory sequence (TRS) sequence is different compared to an intergenic or transcription regulatory sequence (TRS) of a modeled RNA virus.

7. The single-stranded polyribonucleotide of claim 1, wherein the one or more MV-RNA sequences are capable of gene silencing activity of a viral gene.

8. The single-stranded polyribonucleotide of claim 1, wherein the one or more MV-RNA sequences are capable of gene silencing activity of a host gene.

9. The single-stranded polyribonucleotide of claim 2, wherein the one or more aptamer sequences are on one or more surface loops of a polyribonucleotide nanoparticle formed by the single-stranded polyribonucleotide and can bind to a structural protein of a virus.

10. The single-stranded polyribonucleotide of claim 2, wherein the one or more aptamer sequences are on one or more surface loops of a polyribonucleotide nanoparticle formed by the single-stranded polyribonucleotide and can bind to a protein of a host infected with the virus.

11. The single-stranded polyribonucleotide of claim 1, wherein the 5′ leader sequence comprises one or more mRNA coding sequences.

12. The single-stranded polyribonucleotide of claim 1, wherein the 5′ leader sequence comprises one or more non-coding sequences.

13. The single-stranded polyribonucleotide of claim 2, wherein the one or more aptamer sequences are on one or more surface loops of a polyribonucleotide nanoparticle formed by the single-stranded polyribonucleotide and can bind to a protein co-expressed by the single-stranded polynucleotide.

14. The single-stranded polyribonucleotide of claim 1, wherein the RNA virus belongs to a Picornaviridae, Astroviridae, Caliciviridae, Hepeviridae, Flaviviridae, Togaviridae, Arteriviridae, or Coronaviridae virus family.

15. The single-stranded polyribonucleotide of claim 4, wherein the RNA virus is hepatitis C virus, West Nile virus, dengue virus, SARS virus, MERS virus, or SARS-CoV-2 virus.

16. A virion-like nanoparticle comprising:
(I) a polyribonucleotide nanoparticle self-forming from the single-stranded polyribonucleotide of claim 1 and
(II) one or more proteins coating the polyribonucleotide nanoparticle.

17. The virion-like nanoparticle of claim 16, wherein the one or more proteins is a capsid protein, another structural protein of a modeled RNA virus, a membrane protein, an envelope protein, an ecto (spike) protein, an endo protein (nucleocapsid) or a combination thereof.

18. The virion-like nanoparticle of claim 16, wherein the one or more proteins are a Picornaviridae, Astroviridae, Caliciviridae, Hepeviridae, Flaviviridae, Togaviridae, Arteriviridae, or Coronaviridae viral protein.

19. A method of treating a disease by administering to a patient in need thereof a therapeutically effective amount of a virion-like nanoparticle comprising:
(I) a polyribonucleotide nanoparticle self-forming from a single stranded polyribonucleotide comprising:
(a) a 5 ′leader sequence;
(b) a leader transcription regulatory sequence (TRS) following the 3′ end of the 5 ′leader sequence;
(c) two or more MV-RNA/aptamer sequences, following the 3 ′end of the leader TRS, each comprising (i) one or more MV-RNA sequences and (ii) one or more aptamer sequences;
(d) one or more internal TRS linking each of the MV-RNA/aptamer sequences; and
(e) a 3 ′untranslated region (UTR) sequence following the two or more MV-RNA/aptamer sequences;
wherein the 5 ′leader sequence, the leader TRS, the internal TRS, and the 3 ′UTR sequence are functional in the presence of an RNA polymerase (RdRp) of an RNA virus; and the leader TRS and the internal TRS are different from the TRS of the RNA virus; and
(II) one or more viral proteins coating the polyribonucleotide nanoparticle.

20. A method of crop protection comprising administering to an insect an effective amount of a composition comprising the virion-like nanoparticle of claim 16.


SEQUENCE LISTING (107 sequences)

SEQ ID NO: 1 - RNA, 29903 nt, SARS-2 coronavirus
  5′UTR (1)..(265)
  promoter (68)..(75): Leader TRS or TRS-L
  CDS (11843)..(12091): RdRp co-factor gene nsp7, works in complex with nsp12
  CDS (12092)..(12685): RdRp co-factor gene nsp8, works in complex with nsp12
  CDS (13442)..(16237): RdRp gene nsp12
  CDS (18040)..(19620): RdRp proof-reading gene nsp14, works in complex with nsp12
  promoter (21554)..(21561): Body TRS (B-TRS)
  misc_RNA (21556)..(21561): Conserved Sequence (CS)
  promoter (25385)..(25390): Example of other Body TRS (B-TRS)
  promoter (26237)..(26242): TRS (B-TRS)
  promoter (26473)..(26478): TRS (B-TRS)
  promoter (27041)..(27046): TRS (B-TRS)
  promoter (27886)..(27893): TRS (B-TRS)
  3′UTR (29675)..(29903)
auuaaagguuuauaccuucccagguaacaaaccaaccaacuuucgaucucuuguagaucuguucucuaaacgaacuuuaaaaucuguguggcugucacucggcugcaugcuuagugcacucacgcaguauaauuaauaacuaauuacugucguugacaggacacgaguaacucgucuaucuucugcaggcugcuuacgguuucguccguguugcagccgaucaucagcacaucuagguuucguccgggugugaccgaaagguaagauggagagccuugucccugguuucaacgagaaaacacacguccaacucaguuugccuguuuuacagguucgcgacgugcucguacguggcuuuggagacuccguggaggaggucuuaucagaggcacgucaacaucuuaaagauggcacuuguggcuuaguagaaguugaaaaaggcguuuugccucaacuugaacagcccuauguguucaucaaacguucggaugcucgaacugcaccucauggucauguuaugguugagcugguagcagaacucgaaggcauucaguacggucguaguggugagacacuugguguccuugucccucaugugggcgaaauaccaguggcuuaccgcaagguucuucuucguaagaacgguaauaaaggagcugguggccauaguuacggcgccgaucuaaagucauuugacuuaggcgacgagcuuggcacugauccuuaugaagauuuucaagaaaacuggaacacuaaacauagcagugguguuacccgugaacucaugcgugagcuuaacggaggggcauacacucgcuaugucgauaacaacuucuguggcccugauggcuacccucuugagugcauuaaagaccuucuagcacgugcugguaaagcuucaugcacuuuguccgaacaacuggacuuuauugacacuaagagggguguauacugcugccgugaacaugagcaugaaauugcuugguacacggaacguucugaaaagagcuaugaauugcagacaccuuuugaaauuaaauuggcaaagaaauuugacaccuucaauggggaauguccaaauuuuguauuucccuuaaauuccauaaucaagacuauucaaccaaggguugaaaagaaaaagcuugauggcuuuauggguagaauucgaucugucuauccaguugcgucaccaaaugaaugcaaccaaaugugccuuucaacucucaugaagugugaucauuguggugaaacuucauggcagacgggcgauuuuguuaaagccacuugcgaauuuuguggcacugagaauuugacuaaagaaggugccacuacuugugguuacuuaccccaaaaugcuguuguuaaaauuuauuguccagcaugucacaauucagaaguaggaccugagcauagucuugccgaauaccauaaugaaucuggcuugaaaaccauucuucguaaggguggucgcacuauugccuuuggaggcuguguguucucuuauguugguugccauaacaagugugccuauuggguuccacgugcuagcgcuaacauagguuguaaccauacagguguuguuggagaagguuccgaaggucuuaaugacaaccuucuugaaauacuccaaaaagagaaagucaacaucaauauuguuggugacuuuaaacuuaaugaagagaucgccauuauuuuggcaucuuuuucugcuuccacaagugcuuuuguggaaacugugaaagguuuggauuauaaagcauucaaacaaauuguugaauccugugguaauuuuaaaguuacaaaaggaaaagcuaaaaaaggugccuggaauauuggugaacagaaaucaauacugaguccucuuuaugcauuugcaucagaggcugcucguguuguacgaucaauuuucucccgcacucuugaaacugcucaaaauucugugcguguuuuacagaaggccgcuauaacaauacuagauggaauuucacaguauucacugagacucauugaugcuaugauguucacaucugauuuggcuacuaacaaucuaguuguaauggccuacauuacaggugguguuguucaguugacuucgcaguggcuaacuaacaucuuuggcacuguuuaugaaaaacucaaacccguccuugauuggcuugaagagaaguuuaaggaagguguagaguuucuuagagacgguugggaaauuguuaaauuuaucucaaccugugcuugugaaauugucgguggacaaauugucaccugugcaaaggaaauuaaggagaguguucagacauucuuuaagcuuguaaauaaauuuuuggcuuugugugcugacucuaucauuauugguggagcuaaacuuaaagccuugaauuuaggugaaacauuugucacgcacucaaagggauuguacagaaaguguguuaaauccagagaagaaacuggccuacucaugccucuaaaagccccaaaagaaauuaucuucuuagagggagaaacacuucccacagaaguguuaacagaggaaguugucuugaaaacuggugauuuacaaccauuagaacaaccuacuagugaagcuguugaagcuccauugguugguacaccaguuuguauuaacgggcuuauguugcucgaaaucaaagacacagaaaaguacugugcccuugcaccuaauaugaugguaacaaacaauaccuucacacucaaaggcggugcaccaacaaagguuacuuuuggugaugacacugugauagaagugcaagguuacaagagugugaauaucacuuuugaacuugaugaaaggauugauaaaguacuuaaugagaagugcucugccuauacaguugaacucgguacagaaguaaaugaguucgccuguguuguggcagaugcugucauaaaaacuuugcaaccaguaucugaauuacuuacaccacugggcauugauuuagaugaguggaguauggcuacauacuacuuauuugaugagucuggugaguuuaaauuggcuucacauauguauuguucuuucuacccuccagaugaggaugaagaagaaggugauugugaagaagaagaguuugagccaucaacucaauaugaguaugguacugaagaugauuaccaagguaaaccuuuggaauuuggugccacuucugcugcucuucaaccugaagaagagcaagaagaagauugguuagaugaugauagucaacaaacuguuggucaacaagacggcagugaggacaaucagacaacuacuauucaaacaauuguugagguucaaccucaauuagagauggaacuuacaccaguuguucagacuauugaagugaauaguuuuagugguuauuuaaaacuuacugacaauguauacauuaaaaaugcagacauuguggaagaagcuaaaaagguaaaaccaacagugguuguuaaugcagccaauguuuaccuuaaacauggaggagguguugcaggagccuuaaauaaggcuacuaacaaugccaugcaaguugaaucugaugauuacauagcuacuaauggaccacuuaaagugggugguaguuguguuuuaagcggacacaaucuugcuaaacacugucuucauguugucggcccaaauguuaacaaaggugaagacauucaacuucuuaagagugcuuaugaaaauuuuaaucagcacgaaguucuacuugcaccauuauuaucagcugguauuuuuggugcugacccuauacauucuuuaagaguuuguguagauacuguucgcacaaaugucuacuuagcugucuuugauaaaaaucucuaugacaaacuuguuucaagcuuuuuggaaaugaagagugaaaagcaaguugaacaaaagaucgcugagauuccuaaagaggaaguuaagccauuuauaacugaaaguaaaccuucaguugaacagagaaaacaagaugauaagaaaaucaaagcuuguguugaagaaguuacaacaacucuggaagaaacuaaguuccucacagaaaacuuguuacuuuauauugacauuaauggcaaucuucauccagauucugccacucuuguuagugacauugacaucacuuucuuaaagaaagaugcuccauauauagugggugauguuguucaagaggguguuuuaacugcugugguuauaccuacuaaaaaggcugguggcacuacugaaaugcuagcgaaagcuuugagaaaagugccaacagacaauuauauaaccacuuacccgggucaggguuuaaaugguuacacuguagaggaggcaaagacagugcuuaaaaaguguaaaagugccuuuuacauucuaccaucuauuaucucuaaugagaagcaagaaauucuuggaacuguuucuuggaauuugcgagaaaugcuugcacaugcagaagaaacacgcaaauuaaugccugucuguguggaaacuaaagccauaguuucaacuauacagcguaaauauaaggguauuaaaauacaagagggugugguugauuauggugcuagauuuuacuuuuacaccaguaaaacaacuguagcgucacuuaucaacacacuuaacgaucuaaaugaaacucuuguuacaaugccacuuggcuauguaacacauggcuuaaauuuggaagaagcugcucgguauaugagaucucucaaagugccagcuacaguuucuguuucuucaccugaugcuguuacagcguauaaugguuaucuuacuucuucuucuaaaacaccugaagaacauuuuauugaaaccaucucacuugcugguuccuauaaagauugguccuauucuggacaaucuacacaacuagguauagaauuucuuaagagaggugauaaaaguguauauuacacuaguaauccuaccacauuccaccuagauggugaaguuaucaccuuugacaaucuuaagacacuucuuucuuugagagaagugaggacuauuaagguguuuacaacaguagacaacauuaaccuccacacgcaaguuguggacaugucaaugacauauggacaacaguuugguccaacuuauuuggauggagcugauguuacuaaaauaaaaccucauaauucacaugaagguaaaacauuuuauguuuuaccuaaugaugacacucuacguguugaggcuuuugaguacuaccacacaacugauccuaguuuucuggguagguacaugucagcauuaaaucacacuaaaaaguggaaauacccacaaguuaaugguuuaacuucuauuaaaugggcagauaacaacuguuaucuugccacugcauuguuaacacuccaacaaauagaguugaaguuuaauccaccugcucuacaagaugcuuauuacagagcaagggcuggugaagcugcuaacuuuugugcacuuaucuuagccuacuguaauaagacaguaggugaguuaggugauguuagagaaacaaugaguuacuuguuucaacaugccaauuuagauucuugcaaaagagucuugaacgugguguguaaaacuuguggacaacagcagacaacccuuaaggguguagaagcuguuauguacaugggcacacuuucuuaugaacaauuuaagaaagguguucagauaccuuguacgugugguaaacaagcuacaaaauaucuaguacaacaggagucaccuuuuguuaugaugucagcaccaccugcucaguaugaacuuaagcaugguacauuuacuugugcuagugaguacacugguaauuaccaguguggucacuauaaacauauaacuucuaaagaaacuuuguauugcauagacggugcuuuacuuacaaaguccucagaauacaaagguccuauuacggauguuuucuacaaagaaaacaguuacacaacaaccauaaaaccaguuacuuauaaauuggaugguguuguuuguacagaaauugacccuaaguuggacaauuauuauaagaaagacaauucuuauuucacagagcaaccaauugaucuuguaccaaaccaaccauauccaaacgcaagcuucgauaauuuuaaguuuguaugugauaauaucaaauuugcugaugauuuaaaccaguuaacugguuauaagaaaccugcuucaagagagcuuaaaguuacauuuuucccugacuuaaauggugaugugguggcuauugauuauaaacacuacacacccucuuuuaagaaaggagcuaaauuguuacauaaaccuauuguuuggcauguuaacaaugcaacuaauaaagccacguauaaaccaaauaccugguguauacguugucuuuggagcacaaaaccaguugaaacaucaaauucguuugauguacugaagucagaggacgcgcagggaauggauaaucuugccugcgaagaucuaaaaccagucucugaagaaguaguggaaaauccuaccauacagaaagacguucuugaguguaaugugaaaacuaccgaaguuguaggagacauuauacuuaaaccagcaaauaauaguuuaaaaauuacagaagagguuggccacacagaucuaauggcugcuuauguagacaauucuagucuuacuauuaagaaaccuaaugaauuaucuagaguauuagguuugaaaacccuugcuacucaugguuuagcugcuguuaauagugucccuugggauacuauagcuaauuaugcuaagccuuuucuuaacaaaguuguuaguacaacuacuaacauaguuacacgguguuuaaaccguguuuguacuaauuauaugccuuauuucuuuacuuuauugcuacaauuguguacuuuuacuagaaguacaaauucuagaauuaaagcaucuaugccgacuacuauagcaaagaauacuguuaagagugucgguaaauuuugucuagaggcuucauuuaauuauuugaagucaccuaauuuuucuaaacugauaaauauuauaauuugguuuuuacuauuaaguguuugccuagguucuuuaaucuacucaaccgcugcuuuagguguuuuaaugucuaauuuaggcaugccuucuuacuguacugguuacagagaaggcuauuugaacucuacuaaugucacuauugcaaccuacuguacugguucuauaccuuguaguguuugucuuagugguuuagauucuuuagacaccuauccuucuuuagaaacuauacaaauuaccauuucaucuuuuaaaugggauuuaacugcuuuuggcuuaguugcagagugguuuuuggcauauauucuuuucacuagguuuuucuauguacuuggauuggcugcaaucaugcaauuguuuuucagcuauuuugcaguacauuuuauuaguaauucuuggcuuaugugguuaauaauuaaucuuguacaaauggccccgauuucagcuaugguuagaauguacaucuucuuugcaucauuuuauuauguauggaaaaguuaugugcauguuguagacgguuguaauucaucaacuuguaugauguguuacaaacguaauagagcaacaagagucgaauguacaacuauuguuaaugguguuagaagguccuuuuaugucuaugcuaauggagguaaaggcuuuugcaaacuacacaauuggaauuguguuaauugugauacauucugugcugguaguacauuuauuagugaugaaguugcgagagacuugucacuacaguuuaaaagaccaauaaauccuacugaccagucuucuuacaucguugauaguguuacagugaagaaugguuccauccaucuuuacuuugauaaagcuggucaaaagacuuaugaaagacauucucucucucauuuuguuaacuuagacaaccugagagcuaauaacacuaaagguucauugccuauuaauguuauaguuuuugaugguaaaucaaaaugugaagaaucaucugcaaaaucagcgucuguuuacuacagucagcuuaugugucaaccuauacuguuacuagaucaggcauuagugucugauguuggugauagugcggaaguugcaguuaaaauguuugaugcuuacguuaauacguuuucaucaacuuuuaacguaccaauggaaaaacucaaaacacuaguugcaacugcagaagcugaacuugcaaagaauguguccuuagacaaugucuuaucuacuuuuauuucagcagcucggcaaggguuuguugauucagauguagaaacuaaagauguuguugaaugucuuaaauugucacaucaaucugacauagaaguuacuggcgauaguuguaauaacuauaugcucaccuauaacaaaguugaaaacaugacaccccgugaccuuggugcuuguauugacuguagugcgcgucauauuaaugcgcagguagcaaaaagucacaacauugcuuugauauggaacguuaaagauuucaugucauugucugaacaacuacgaaaacaaauacguagugcugcuaaaaagaauaacuuaccuuuuaaguugacaugugcaacuacuagacaaguuguuaauguuguaacaacaaagauagcacuuaagggugguaaaauuguuaauaauugguugaagcaguuaauuaaaguuacacuuguguuccuuuuuguugcugcuauuuucuauuuaauaacaccuguucaugucaugucuaaacauacugacuuuucaagugaaaucauaggauacaaggcuauugaugguggugucacucgugacauagcaucuacagauacuuguuuugcuaacaaacaugcugauuuugacacaugguuuagccagcguggugguaguuauacuaaugacaaagcuugcccauugauugcugcagucauaacaagagaaguggguuuugucgugccugguuugccuggcacgauauuacgcacaacuaauggugacuuuuugcauuucuuaccuagaguuuuuagugcaguugguaacaucuguuacacaccaucaaaacuuauagaguacacugacuuugcaacaucagcuuguguuuuggcugcugaauguacaauuuuuaaagaugcuucugguaagccaguaccauauuguuaugauaccaauguacuagaagguucuguugcuuaugaaaguuuacgcccugacacacguuaugugcucauggauggcucuauuauucaauuuccuaacaccuaccuugaagguucuguuagagugguaacaacuuuugauucugaguacuguaggcacggcacuugugaaagaucagaagcugguguuuguguaucuacuagugguagauggguacuuaacaaugauuauuacagaucuuuaccaggaguuuucugugguguagaugcuguaaauuuacuuacuaauauguuuacaccacuaauucaaccuauuggugcuuuggacauaucagcaucuauaguagcuggugguauuguagcuaucguaguaacaugccuugccuacuauuuuaugagguuuagaagagcuuuuggugaauacagucauguaguugccuuuaauacuuuacuauuccuuaugucauucacuguacucuguuuaacaccaguuuacucauucuuaccugguguuuauucuguuauuuacuuguacuugacauuuuaucuuacuaaugauguuucuuuuuuagcacauauucaguggaugguuauguucacaccuuuaguaccuuucuggauaacaauugcuuauaucauuuguauuuccacaaagcauuucuauugguucuuuaguaauuaccuaaagagacguguagucuuuaaugguguuuccuuuaguacuuuugaagaagcugcgcugugcaccuuuuuguuaaauaaagaaauguaucuaaaguugcguagugaugugcuauuaccucuuacgcaauauaauagauacuuagcucuuuauaauaaguacaaguauuuuaguggagcaauggauacaacuagcuacagagaagcugcuuguugucaucucgcaaaggcucucaaugacuucaguaacucagguucugauguucuuuaccaaccaccacaaaccucuaucaccucagcuguuuugcagagugguuuuagaaaaauggcauucccaucugguaaaguugaggguuguaugguacaaguaacuugugguacaacuacacuuaacggucuuuggcuugaugacguaguuuacuguccaagacaugugaucugcaccucugaagacaugcuuaacccuaauuaugaagauuuacucauucguaagucuaaucauaauuucuugguacaggcugguaauguucaacucaggguuauuggacauucuaugcaaaauuguguacuuaagcuuaagguugauacagccaauccuaagacaccuaaguauaaguuuguucgcauucaaccaggacagacuuuuucaguguuagcuuguuacaaugguucaccaucugguguuuaccaaugugcuaugaggcccaauuucacuauuaaggguucauuccuuaaugguucaugugguaguguugguuuuaacauagauuaugacugugucucuuuuuguuacaugcaccauauggaauuaccaacuggaguucaugcuggcacagacuuagaagguaacuuuuauggaccuuuuguugacaggcaaacagcacaagcagcugguacggacacaacuauuacaguuaauguuuuagcuugguuguacgcugcuguuauaaauggagacaggugguuucucaaucgauuuaccacaacucuuaaugacuuuaaccuuguggcuaugaaguacaauuaugaaccucuaacacaagaccauguugacauacuaggaccucuuucugcucaaacuggaauugccguuuuagauaugugugcuucauuaaaagaauuacugcaaaaugguaugaauggacguaccauauuggguagugcuuuauuagaagaugaauuuacaccuuuugauguuguuagacaaugcucagguguuacuuuccaaagugcagugaaaagaacaaucaaggguacacaccacugguuguuacucacaauuuugacuucacuuuuaguuuuaguccagaguacucaauggucuuuguucuuuuuuuuguaugaaaaugccuuuuuaccuuuugcuauggguauuauugcuaugucugcuuuugcaaugauguuugucaaacauaagcaugcauuucucuguuuguuuuuguuaccuucucuugccacuguagcuuauuuuaauauggucuauaugccugcuaguugggugaugcguauuaugacaugguuggauaugguugauacuaguuugucugguuuuaagcuaaaagacuguguuauguaugcaucagcuguaguguuacuaauccuuaugacagcaagaacuguguaugaugauggugcuaggagaguguggacacuuaugaaugucuugacacucguuuauaaaguuuauuaugguaaugcuuuagaucaagccauuuccaugugggcucuuauaaucucuguuacuucuaacuacucagguguaguuacaacugucauguuuuuggccagagguauuguuuuuauguguguugaguauugcccuauuuucuucauaacugguaauacacuucaguguauaaugcuaguuuauuguuucuuaggcuauuuuuguacuuguuacuuuggccucuuuuguuuacucaaccgcuacuuuagacugacucuugguguuuaugauuacuuaguuucuacacaggaguuuagauauaugaauucacagggacuacucccacccaagaauagcauagaugccuucaaacucaacauuaaauuguuggguguugguggcaaaccuuguaucaaaguagccacuguacagucuaaaaugucagauguaaagugcacaucaguagucuuacucucaguuuugcaacaacucagaguagaaucaucaucuaaauugugggcucaauguguccaguuacacaaugacauucucuuagcuaaagauacuacugaagccuuugaaaaaaugguuucacuacuuucuguuuugcuuuccaugcagggugcuguagacauaaacaagcuuugugaagaaaugcuggacaacagggcaaccuuacaagcuauagccucagaguuuaguucccuuccaucauaugcagcuuuugcuacugcucaagaagcuuaugagcaggcuguugcuaauggugauucugaaguuguucuuaaaaaguugaagaagucuuugaauguggcuaaaucugaauuugaccgugaugcagccaugcaacguaaguuggaaaagauggcugaucaagcuaugacccaaauguauaaacaggcuagaucugaggacaagagggcaaaaguuacuagugcuaugcagacaaugcuuuucacuaugcuuagaaaguuggauaaugaugcacucaacaacauuaucaacaaugcaagagaugguuguguucccuugaacauaauaccucuuacaacagcagccaaacuaaugguugucauaccagacuauaacacauauaaaaauacgugugaugguacaacauuuacuuaugcaucagcauugugggaaauccaacagguuguagaugcagauaguaaaauuguucaacuuagugaaauuaguauggacaauucaccuaauuuagcauggccucuuauuguaacagcuuuaagggccaauucugcugucaaauuacagaauaaugagcuuaguccuguugcacuacgacagaugucuugugcugccgguacuacacaaacugcuugcacugaugacaaugcguuagcuuacuacaacacaacaaagggagguagguuuguacuugcacuguuauccgauuuacaggauuugaaaugggcuagauucccuaagagugauggaacugguacuaucuauacagaacuggaaccaccuuguagguuuguuacagacacaccuaaagguccuaaagugaaguauuuauacuuuauuaaaggauuaaacaaccuaaauagagguaugguacuugguaguuuagcugccacaguacgucuacaagcugguaaugcaacagaagugccugccaauucaacuguauuaucuuucugugcuuuugcuguagaugcugcuaaagcuuacaaagauuaucuagcuagugggggacaaccaaucacuaauuguguuaagauguuguguacacacacugguacuggucaggcaauaacaguuacaccggaagccaauauggaucaagaauccuuugguggugcaucguguugucuguacugccguugccacauagaucauccaaauccuaaaggauuuugugacuuaaaagguaaguauguacaaauaccuacaacuugugcuaaugacccuguggguuuuacacuuaaaaacacagucuguaccgucugcgguauguggaaagguuauggcuguaguugugaucaacuccgcgaacccaugcuucagucagcugaugcacaaucguuuuuaaacggguuugcgguguaagugcagcccgucuuacaccgugcggcacaggcacuaguacugaugucguauacagggcuuuugacaucuacaaugauaaaguagcugguuuugcuaaauuccuaaaaacuaauuguugucgcuuccaagaaaaggacgaagaugacaauuuaauugauucuuacuuuguaguuaagagacacacuuucucuaacuaccaacaugaagaaacaauuuauaauuuacuuaaggauuguccagcuguugcuaaacaugacuucuuuaaguuuagaauagacggugacaugguaccacauauaucacgucaacgucuuacuaaauacacaauggcagaccucgucuaugcuuuaaggcauuuugaugaagguaauugugacacauuaaaagaaauacuugucacauacaauuguugugaugaugauuauuucaauaaaaaggacugguaugauuuuguagaaaacccagauauauuacgcguauacgccaacuuaggugaacguguacgccaagcuuuguuaaaaacaguacaauucugugaugccaugcgaaaugcugguauuguugguguacugacauuagauaaucaagaucucaaugguaacugguaugauuucggugauuucauacaaaccacgccagguaguggaguuccuguuguagauucuuauuauucauuguuaaugccuauauuaaccuugaccagggcuuuaacugcagagucacauguugacacugacuuaacaaagccuuacauuaagugggauuuguuaaaauaugacuucacggaagagagguuaaaacucuuugaccguuauuuuaaauauugggaucagacauaccacccaaauuguguuaacuguuuggaugacagaugcauucugcauugugcaaacuuuaauguuuuauucucuacaguguucccaccuacaaguuuuggaccacuagugagaaaaauauuuguugaugguguuccauuuguaguuucaacuggauaccacuucagagagcuagguguuguacauaaucaggauguaaacuuacauagcucuagacuuaguuuuaaggaauuacuuguguaugcugcugacccugcuaugcacgcugcuucugguaaucuauuacuagauaaacgcacuacgugcuuuucaguagcugcacuuacuaacaauguugcuuuucaaacugucaaacccgguaauuuuaacaaagacuucuaugacuuugcugugucuaaggguuucuuuaaggaaggaaguucuguugaauuaaaacacuucuucuuugcucaggaugguaaugcugcuaucagcgauuaugacuacuaucguuauaaucuaccaacaaugugugauaucagacaacuacuauuuguaguugaaguuguugauaaguacuuugauuguuacgaugguggcuguauuaaugcuaaccaagucaucgucaacaaccuagacaaaucagcugguuuuccauuuaauaaaugggguaaggcuagacuuuauuaugauucaaugaguuaugaggaucaagaugcacuuuucgcauauacaaaacguaaugucaucccuacuauaacucaaaugaaucuuaaguaugccauuagugcaaagaauagagcucgcaccguagcuggugucucuaucuguaguacuaugaccaauagacaguuucaucaaaaauuauugaaaucaauagccgccacuagaggagcuacuguaguaauuggaacaagcaaauucuauggugguuggcacaacauguuaaaaacuguuuauagugauguagaaaacccucaccuuauggguugggauuauccuaaaugugauagagccaugccuaacaugcuuagaauuauggccucacuuguucuugcucgcaaacauacaacguguuguagcuugucacaccguuucuauagauuagcuaaugagugugcucaaguauugagugaaauggucauguguggcgguucacuauauguuaaaccagguggaaccucaucaggagaugccacaacugcuuaugcuaauaguguuuuuaacauuugucaagcugucacggccaauguuaaugcacuuuuaucuacugaugguaacaaaauugccgauaaguauguccgcaauuuacaacacagacuuuaugagugucucuauagaaauagagauguugacacagacuuugugaaugaguuuuacgcauauuugcguaaacauuucucaaugaugauacucucugacgaugcuguuguguguuucaauagcacuuaugcaucucaaggucuaguggcuagcauaaagaacuuuaagucaguucuuuauuaucaaaacaauguuuuuaugucugaagcaaaauguuggacugagacugaccuuacuaaaggaccucaugaauuuugcucucaacauacaaugcuaguuaaacagggugaugauuauguguaccuuccuuacccagauccaucaagaauccuaggggccggcuguuuuguagaugauaucguaaaaacagaugguacacuuaugauugaacgguucgugucuuuagcuauagaugcuuacccacuuacuaaacauccuaaucaggaguaugcugaugucuuucauuuguacuuacaauacauaagaaagcuacaugaugaguuaacaggacacauguuagacauguauucuguuaugcuuacuaaugauaacacuucaagguauugggaaccugaguuuuaugaggcuauguacacaccgcauacagucuuacaggcuguuggggcuuguguucuuugcaauucacagacuucauuaagauguggugcuugcauacguagaccauucuuauguuguaaaugcuguuacgaccaugucauaucaacaucacauaaauuagucuugucuguuaauccguauguuugcaaugcuccagguugugaugucacagaugugacucaacuuuacuuaggagguaugagcuauuauuguaaaucacauaaaccacccauuaguuuuccauugugugcuaauggacaaguuuuugguuuauauaaaaauacauguguugguagcgauaauguuacugacuuuaaugcaauugcaacaugugacuggacaaaugcuggugauuacauuuuagcuaacaccuguacugaaagacucaagcuuuuugcagcagaaacgcucaaagcuacugaggagacauuuaaacugucuuaugguauugcuacuguacgugaagugcugucugacagagaauuacaucuuucaugggaaguugguaaaccuagaccaccacuuaaccgaaauuaugucuuuacugguuaucguguaacuaaaaacaguaaaguacaaauaggagaguacaccuuugaaaaaggugacuauggugaugcuguuguuuaccgagguacaacaacuuacaaauuaaauguuggugauuauuuugugcugacaucacauacaguaaugccauuaagugcaccuacacuagugccacaagagcacuauguuagaauuacuggcuuauacccaacacucaauaucucagaugaguuuucuagcaauguugcaaauuaucaaaagguugguaugcaaaaguauucuacacuccagggaccaccugguacugguaagagucauuuugcuauuggccuagcucucuacuacccuucugcucgcauaguguauacagcuugcucucaugccgcuguugaugcacuaugugagaaggcauuaaaauauuugccuauagauaaauguaguagaauuauaccugcacgugcucguguagaguguuuugauaaauucaaagugaauucaacauuagaacaguaugucuuuuguacuguaaaugcauugccugagacgacagcagauauaguugucuuugaugaaauuucaauggccacaaauuaugauuugaguguugucaaugccagauuacgugcuaagcacuauguguacauuggcgacccugcucaauuaccugcaccacgcacauugcuaacuaagggcacacuagaaccagaauauuucaauucaguguguagacuuaugaaaacuauagguccagacauguuccucggaacuugucggcguuguccugcugaaauuguugacacugugagugcuuugguuuaugauaauaagcuuaaagcacauaaagacaaaucagcucaaugcuuuaaaauguuuuauaaggguguuaucacgcaugauguuucaucugcaauuaacaggccacaaauaggcgugguaagagaauuccuuacacguaacccugcuuggagaaaagcugucuuuauuucaccuuauaauucacagaaugcuguagccucaaagauuuugggacuaccaacucaaacuguugauucaucacagggcucagaauaugacuaugucauauucacucaaaccacugaaacagcucacucuuguaauguaaacagauuuaauguugcuauuaccagagcaaaaguaggcauacuuugcauaaugucugauagagaccuuuaugacaaguugcaauuuacaagucuugaaauuccacguaggaauguggcaacuuuacaagcugaaaauguaacaggacucuuuaaagauuguaguaagguaaucacuggguuacauccuacacaggcaccuacacaccucaguguugacacuaaauucaaaacugaagguuuauguguugacauaccuggcauaccuaaggacaugaccuauagaagacucaucucuaugauggguuuuaaaaugaauuaucaaguuaaugguuacccuaacauguuuaucacccgcgaagaagcuauaagacauguacgugcauggauuggcuucgaugucgaggggugucaugcuacuagagaagcuguugguaccaauuuaccuuuacagcuagguuuuucuacagguguuaaccuaguugcuguaccuacagguuauguugauacaccuaauaauacagauuuuuccagaguuagugcuaaaccaccgccuggagaucaauuuaaacaccucauaccacuuauguacaaaggacuuccuuggaauguagugcguauaaagauuguacaaauguuaagugacacacuuaaaaaucucucugacagagucguauuugucuuaugggcacauggcuuugaguugacaucuaugaaguauuuugugaaaauaggaccugagcgcaccuguugucuaugugauagacgugccacaugcuuuuccacugcuucagacacuuaugccuguuggcaucauucuauuggauuugauuacgucuauaauccguuuaugauugauguucaacaaugggguuuuacagguaaccuacaaagcaaccaugaucuguauugucaaguccaugguaaugcacauguagcuaguugugaugcaaucaugacuaggugucuagcuguccacgagugcuuuguuaagcguguugacuggacuauugaauauccuauaauuggugaugaacugaagauuaaugcggcuuguagaaagguucaacacaugguuguuaaagcugcauuauuagcagacaaauucccaguucuucacgacauugguaacccuaaagcuauuaaguguguaccucaagcugauguagaauggaaguucuaugaugcacagccuuguagugacaaagcuuauaaaauagaagaauuauucuauucuuaugccacacauucugacaaauucacagaugguguaugccuauuuuggaauugcaaugucgauagauauccugcuaauuccauuguuuguagauuugacacuagagugcuaucuaaccuuaacuugccugguugugaugguggcaguuuguauguaaauaaacaugcauuccacacaccagcuuuugauaaaagugcuuuuguuaauuuaaaacaauuaccauuuuucuauuacucugacaguccaugugagucucauggaaaacaaguagugucagauauagauuauguaccacuaaagucugcuacguguauaacacguugcaauuuagguggugcugucuguagacaucaugcuaaugaguacagauuguaucucgaugcuuauaacaugaugaucucagcuggcuuuagcuuguggguuuacaaacaauuugauacuuauaaccucuggaacacuuuuacaagacuucagaguuuagaaaauguggcuuuuaauguuguaaauaagggacacuuugauggacaacagggugaaguaccaguuucuaucauuaauaacacuguuuacacaaaaguugaugguguugauguagaauuguuugaaaauaaaacaacauuaccuguuaauguagcauuugagcuuugggcuaagcgcaacauuaaaccaguaccagaggugaaaauacucaauaauuuggguguggacauugcugcuaauacugugaucugggacuacaaaagagaugcuccagcacauauaucuacuauugguguuuguucuaugacugacauagccaagaaaccaacugaaacgauuugugcaccacucacugucuuuuuugaugguagaguugauggucaaguagacuuauuuagaaaugcccguaaugguguucuuauuacagaagguaguguuaaagguuuacaaccaucuguaggucccaaacaagcuagucuuaauggagucacauuaauuggagaagccguaaaaacacaguucaauuauuauaagaaaguugaugguguuguccaacaauuaccugaaacuuacuuuacucagaguagaaauuuacaagaauuuaaacccaggagucaaauggaaauugauuucuuagaauuagcuauggaugaauucauugaacgguauaaauuagaaggcuaugccuucgaacauaucguuuauggagauuuuagucauagucaguuaggugguuuacaucuacugauuggacuagcuaaacguuuuaaggaaucaccuuuugaauuagaagauuuuauuccuauggacaguacaguuaaaaacuauuucauaacagaugcgcaaacagguucaucuaaguguguguguucuguuauugauuuauuacuugaugauuuuguugaaauaauaaaaucccaagauuuaucuguaguuucuaagguugucaaagugacuauugacuauacagaaauuucauuuaugcuuugguguaaagauggccauguagaaacauuuuacccaaaauuacaaucuagucaagcguggcaaccggguguugcuaugccuaaucuuuacaaaaugcaaagaaugcuauuagaaaagugugaccuucaaaauuauggugauagugcaacauuaccuaaaggcauaaugaugaaugucgcaaaauauacucaacugugucaauauuuaaacacauuaacauuagcuguacccuauaauaugagaguuauacauuuuggugcugguucugauaaaggaguugcaccagguacagcuguuuuaagacagugguugccuacggguacgcugcuugucgauucagaucuuaaugacuuugucucugaugcagauucaacuuugauuggugauugugcaacuguacauacagcuaauaaaugggaucucauuauuagugauauguacgacccuaagacuaaaaauguuacaaaagaaaaugacucuaaagaggguuuuuucacuuacauuuguggguuuauacaacaaaagcuagcucuuggagguuccguggcuauaaagauaacagaacauucuuggaaugcugaucuuuauaagcucaugggacacuucgcaugguggacagccuuuguuacuaaugugaaugcgucaucaucugaagcauuuuuaauuggauguaauuaucuuggcaaaccacgcgaacaaauagaugguuaugucaugcaugcaaauuacauauuuuggaggaauacaaauccaauucaguugucuuccuauucuuuauuugacaugaguaaauuuccccuuaaauuaagggguacugcuguuaugucuuuaaaagaaggucaaaucaaugauaugauuuuaucucuucuuaguaaagguagacuuauaauuagagaaaacaacagaguuguuauuucuagugauguucuuguuaacaacuaaacgaacaauguuuguuuuucuuguuuuauugccacuagucucuagucaguguguuaaucuuacaaccagaacucaauuacccccugcauacacuaauucuuucacacgugguguuuauuacccugacaaaguuuucagauccucaguuuuacauucaacucaggacuuguucuuaccuuucuuuuccaauguuacuugguuccaugcuauacaugucucugggaccaaugguacuaagagguuugauaacccuguccuaccauuuaaugaugguguuuauuuugcuuccacugagaagucuaacauaauaagaggcuggauuuuugguacuacuuuagauucgaagacccagucccuacuuauuguuaauaacgcuacuaauguuguuauuaaagucugugaauuucaauuuuguaaugauccauuuuuggguguuuauuaccacaaaaacaacaaaaguuggauggaaagugaguucagaguuuauucuagugcgaauaauugcacuuuugaauaugucucucagccuuuucuuauggaccuugaaggaaaacaggguaauuucaaaaaucuuagggaauuuguguuuaagaauauugaugguuauuuuaaaauauauucuaagcacacgccuauuaauuuagugcgugaucucccucaggguuuuucggcuuuagaaccauugguagauuugccaauagguauuaacaucacuagguuucaaacuuuacuugcuuuacauagaaguuauuugacuccuggugauucuucuucagguuggacagcuggugcugcagcuuauuauguggguuaucuucaaccuaggacuuuucuauuaaaauauaaugaaaauggaaccauuacagaugcuguagacugugcacuugacccucucucagaaacaaaguguacguugaaauccuucacuguagaaaaaggaaucuaucaaacuucuaacuuuagaguccaaccaacagaaucuauuguuagauuuccuaauauuacaaacuugugcccuuuuggugaaguuuuuaacgccaccagauuugcaucuguuuaugcuuggaacaggaagagaaucagcaacuguguugcugauuauucuguccuauauaauuccgcaucauuuuccacuuuuaaguguuauggagugucuccuacuaaauuaaaugaucucugcuuuacuaaugucuaugcagauucauuuguaauuagaggugaugaagucagacaaaucgcuccagggcaaacuggaaagauugcugauuauaauuauaaauuaccagaugauuuuacaggcugcguuauagcuuggaauucuaacaaucuugauucuaagguuggugguaauuauaauuaccuguauagauuguuuaggaagucuaaucucaaaccuuuugagagagauauuucaacugaaaucuaucaggccgguagcacaccuuguaaugguguugaagguuuuaauuguuacuuuccuuuacaaucauaugguuuccaacccacuaaugguguugguuaccaaccauacagaguaguaguacuuucuuuugaacuucuacaugcaccagcaacuguuuguggaccuaaaaagucuacuaauuugguuaaaaacaaaugugucaauuucaacuucaaugguuuaacaggcacagguguucuuacugagucuaacaaaaaguuucugccuuuccaacaauuuggcagagacauugcugacacuacugaugcuguccgugauccacagacacuugagauucuugacauuacaccauguucuuuugguggugucaguguuauaacaccaggaacaaauacuucuaaccagguugcuguucuuuaucaggauguuaacugcacagaagucccuguugcuauucaugcagaucaacuuacuccuacuuggcguguuuauucuacagguucuaauguuuuucaaacacgugcaggcuguuuaauaggggcugaacaugucaacaacucauaugagugugacauacccauuggugcagguauaugcgcuaguuaucagacucagacuaauucuccucggcgggcacguaguguagcuagucaauccaucauugccuacacuaugucacuuggugcagaaaauucaguugcuuacucuaauaacucuauugccauacccacaaauuuuacuauuaguguuaccacagaaauucuaccagugucuaugaccaagacaucaguagauuguacaauguacauuuguggugauucaacugaaugcagcaaucuuuuguugcaauauggcaguuuuuguacacaauuaaaccgugcuuuaacuggaauagcuguugaacaagacaaaaacacccaagaaguuuuugcacaagucaaacaaauuuacaaaacaccaccaauuaaagauuuuggugguuuuaauuuuucacaaauauuaccagauccaucaaaaccaagcaagaggucauuuauugaagaucuacuuuucaacaaagugacacuugcagaugcuggcuucaucaaacaauauggugauugccuuggugauauugcugcuagagaccucauuugugcacaaaaguuuaacggccuuacuguuuugccaccuuugcucacagaugaaaugauugcucaauacacuucugcacuguuagcggguacaaucacuucugguuggaccuuuggugcaggugcugcauuacaaauaccauuugcuaugcaaauggcuuauagguuuaaugguauuggaguuacacagaauguucucuaugagaaccaaaaauugauugccaaccaauuuaauagugcuauuggcaaaauucaagacucacuuucuuccacagcaagugcacuuggaaaacuucaagauguggucaaccaaaaugcacaagcuuuaaacacgcuuguuaaacaacuuagcuccaauuuuggugcaauuucaaguguuuuaaaugauauccuuucacgucuugacaaaguugaggcugaagugcaaauugauagguugaucacaggcagacuucaaaguuugcagacauaugugacucaacaauuaauuagagcugcagaaaucagagcuucugcuaaucuugcugcuacuaaaaugucagaguguguacuuggacaaucaaaaagaguugauuuuuguggaaagggcuaucaucuuauguccuucccucagucagcaccucaugguguagucuucuugcaugugacuuaugucccugcacaagaaaagaacuucacaacugcuccugccauuugucaugauggaaaagcacacuuuccucgugaaggugucuuuguuucaaauggcacacacugguuuguaacacaaaggaauuuuuaugaaccacaaaucauuacuacagacaacacauuugugucugguaacugugauguuguaauaggaauugucaacaacacaguuuaugauccuuugcaaccugaauuagacucauucaaggaggaguuagauaaauauuuuaagaaucauacaucaccagauguugauuuaggugacaucucuggcauuaaugcuucaguuguaaacauucaaaaagaaauugaccgccucaaugagguugccaagaauuuaaaugaaucucucaucgaucuccaagaacuuggaaaguaugagcaguauauaaaauggccaugguacauuuggcuagguuuuauagcuggcuugauugccauaguaauggugacaauuaugcuuugcuguaugaccaguugcuguaguugucucaagggcuguuguucuuguggauccugcugcaaauuugaugaagacgacucugagccagugcucaaaggagucaaauuacauuacacauaaacgaacuuauggauuuguuuaugagaaucuucacaauuggaacuguaacuuugaagcaaggugaaaucaaggaugcuacuccuucagauuuuguucgcgcuacugcaacgauaccgauacaagccucacucccuuucggauggcuuauuguuggcguugcacuucuugcuguuuuucagagcgcuuccaaaaucauaacccucaaaaagagauggcaacuagcacucuccaaggguguucacuuuguuugcaacuugcuguuguuguuuguaacaguuuacucacaccuuuugcucguugcugcuggccuugaagccccuuuucucuaucuuuaugcuuuagucuacuucuugcagaguauaaacuuuguaagaauaauaaugaggcuuuggcuuugcuggaaaugccguuccaaaaacccauuacuuuaugaugccaacuauuuucuuugcuggcauacuaauuguuacgacuauuguauaccuuacaauaguguaacuucuucaauugucauuacuucaggugauggcacaacaaguccuauuucugaacaugacuaccagauuggugguuauacugaaaaaugggaaucuggaguaaaagacuguguuguauuacacaguuacuucacuucagacuauuaccagcuguacucaacucaauugaguacagacacugguguugaacauguuaccuucuucaucuacaauaaaauuguugaugagccugaagaacauguccaaauucacacaaucgacgguucauccggaguuguuaauccaguaauggaaccaauuuaugaugaaccgacgacgacuacuagcgugccuuuguaagcacaagcugaugaguacgaacuuauguacucauucguuucggaagagacagguacguuaauaguuaauagcguacuucuuuuucuugcuuucgugguauucuugcuaguuacacuagccauccuuacugcgcuucgauugugugcguacugcugcaauauuguuaacgugagucuuguaaaaccuucuuuuuacguuuacucucguguuaaaaaucugaauucuucuagaguuccugaucuucuggucuaaacgaacuaaauauuauauuaguuuuucuguuuggaacuuuaauuuuagccauggcagauuccaacgguacuauuaccguugaagagcuuaaaaagcuccuugaacaauggaaccuaguaauagguuuccuauuccuuacauggauuugucuucuacaauuugccuaugccaacaggaauagguuuuuguauauaauuaaguuaauuuuccucuggcuguuauggccaguaacuuuagcuuguuuugugcuugcugcuguuuacagaauaaauuggaucaccgguggaauugcuaucgcaauggcuugucuuguaggcuugauguggcucagcuacuucauugcuucuuucagacuguuugcgcguacgcguuccauguggucauucaauccagaaacuaacauucuucucaacgugccacuccauggcacuauucugaccagaccgcuucuagaaagugaacucguaaucggagcugugauccuucguggacaucuucguauugcuggacaccaucuaggacgcugugacaucaaggaccugccuaaagaaaucacuguugcuacaucacgaacgcuuucuuauuacaaauugggagcuucgcagcguguagcaggugacucagguuuugcugcauacagucgcuacaggauuggcaacuauaaauuaaacacagaccauuccaguagcagugacaauauugcuuugcuuguacaguaagugacaacagauguuucaucucguugacuuucagguuacuauagcagagauauuacuaauuauuaugaggacuuuuaaaguuuccauuuggaaucuugauuacaucauaaaccucauaauuaaaaauuuaucuaagucacuaacugagaauaaauauucucaauuagaugaagagcaaccaauggagauugauuaaacgaacaugaaaauuauucuuuucuuggcacugauaacacucgcuacuugugagcuuuaucacuaccaagaguguguuagagguacaacaguacuuuuaaaagaaccuugcucuucuggaacauacgagggcaauucaccauuucauccucuagcugauaacaaauuugcacugacuugcuuuagcacucaauuugcuuuugcuuguccugacggcguaaaacacgucuaucaguuacgugccagaucaguuucaccuaaacuguucaucagacaagaggaaguucaagaacuuuacucuccaauuuuucuuauuguugcggcaauaguguuuauaacacuuugcuucacacucaaaagaaagacagaaugauugaacuuucauuaauugacuucuauuugugcuuuuuagccuuucugcuauuccuuguuuuaauuaugcuuauuaucuuuugguucucacuugaacugcaagaucauaaugaaacuugucacgccuaaacgaacaugaaauuucuuguuuucuuaggaaucaucacaacuguagcugcauuucaccaagaauguaguuuacagucauguacucaacaucaaccauauguaguugaugacccguguccuauucacuucuauucuaaaugguauauuagaguaggagcuagaaaaucagcaccuuuaauugaauugugcguggaugaggcugguucuaaaucacccauucaguacaucgauaucgguaauuauacaguuuccuguuuaccuuuuacaauuaauugccaggaaccuaaauuggguagucuuguagugcguuguucguucuaugaagacuuuuuagaguaucaugacguucguguuguuuuagauuucaucuaaacgaacaaacuaaaaugucugauaauggaccccaaaaucagcgaaaugcaccccgcauuacguuugguggacccucagauucaacuggcaguaaccagaauggagaacgcaguggggcgcgaucaaaacaacgucggccccaagguuuacccaauaauacugcgucuugguucaccgcucucacucaacauggcaaggaagaccuuaaauucccucgaggacaaggcguuccaauuaacaccaauagcaguccagaugaccaaauuggcuacuaccgaagagcuaccagacgaauucgugguggugacgguaaaaugaaagaucucaguccaagaugguauuucuacuaccuaggaacugggccagaagcuggacuucccuauggugcuaacaaagacggcaucauauggguugcaacugagggagccuugaauacaccaaaagaucacauuggcacccgcaauccugcuaacaaugcugcaaucgugcuacaacuuccucaaggaacaacauugccaaaaggcuucuacgcagaagggagcagaggcggcagucaagccucuucucguuccucaucacguagucgcaacaguucaagaaauucaacuccaggcagcaguaggggaacuucuccugcuagaauggcuggcaauggcggugaugcugcucuugcuuugcugcugcuugacagauugaaccagcuugagagcaaaaugucugguaaaggccaacaacaacaaggccaaacugucacuaagaaaucugcugcugaggcuucuaagaagccucggcaaaaacguacugccacuaaagcauacaauguaacacaagcuuucggcagacgugguccagaacaaacccaaggaaauuuuggggaccaggaacuaaucagacaaggaacugauuacaaacauuggccgcaaauugcacaauuugcccccagcgcuucagcguucuucggaaugucgcgcauuggcauggaagucacaccuucgggaacgugguugaccuacacaggugccaucaaauuggaugacaaagauccaaauuucaaagaucaagucauuuugcugaauaagcauauugacgcauacaaaacauucccaccaacagagccuaaaaaggacaaaaagaagaaggcugaugaaacucaagccuuaccgcagagacagaagaaacagcaaacugugacucuucuuccugcugcagauuuggaugauuucuccaaacaauugcaacaauccaugagcagugcugacucaacucaggccuaaacucaugcagaccacacaaggcagaugggcuauauaaacguuuucgcuuuuccguuuacgauauauagucuacucuugugcagaaugaauucucguaacuacauagcacaaguagauguaguuaacuuuaaucucacauagcaaucuuuaaucaguguguaacauuagggaggacuugaaagagccaccacauuuucaccgaggccacgcggaguacgaucgaguguacagugaacaaugcuagggagagcugccuauauggaagagcccuaauguguaaaauuaauuuuaguagugcuauccccaugugauuuuaauagcuucuuaggagaaugacaaaaaaaaaaaaaaaaaaaaaaaaaaaaaaaaa

SEQ ID NO: 2 - protein, 83 aa, SARS-2 coronavirus
SKMSDVKCTSVVLLSVLQQLRVESSSKLWAQCVQLHNDILLAKDTTEAFEKMVSLLSVLLSMQGAVDINKLCEEMLDNRATLQ

SEQ ID NO: 3 - protein, 198 aa, SARS-2 coronavirus
AIASEFSSLPSYAAFATAQEAYEQAVANGDSEVVLKKLKKSLNVAKSEFDRDAAMQRKLEKMADQAMTQMYKQARSEDKRAKVTSAMQTMLFTMLRKLDNDALNNIINNARDGCVPLNIIPLTTAAKLMVVIPDYNTYKNTCDGTTFTYASALWEIQQVVDADSKIVQLSEISMDNSPNLAWPLIVTALRANSAVKLQ

SEQ ID NO: 4 - protein, 13 aa, SARS-2 coronavirus
SADAQSFLNGFAV

SEQ ID NO: 5 - protein, 27 aa, SARS-2 coronavirus
VQPVLHRAAQALVLMSYTGLLTSTMIK

SEQ ID NO: 6 - protein, 6 aa, SARS-2 coronavirus
LVLLNS

SEQ ID NO: 7 - protein, 15 aa, SARS-2 coronavirus
KLIVVASKKRTKMTI

SEQ ID NO: 8 - protein, 5 aa, SARS-2 coronavirus
LILTL

SEQ ID NO: 9 - protein, 34 aa, SARS-2 coronavirus
LRDTLSLTTNMKKQFIIYLRIVQLLLNMTSLSLE

SEQ ID NO: 10 - protein, 24 aa, SARS-2 coronavirus
TVTWYHIYHVNVLLNTQWQTSSML

SEQ ID NO: 11 - protein, 10 aa, SARS-2 coronavirus
GILMKVIVTH

SEQ ID NO: 12 - protein, 23 aa, SARS-2 coronavirus
KKYLSHTIVVMMIISIKRTGMIL

SEQ ID NO: 13 - protein, 11 aa, SARS-2 coronavirus
KTQIYYAYTPT

SEQ ID NO: 14 - protein, 8 aa, SARS-2 coronavirus
VNVYAKLC

SEQ ID NO: 15 - protein, 17 aa, SARS-2 coronavirus
KQYNSVMPCEMLVLLVY

SEQ ID NO: 16 - protein, 26 aa, SARS-2 coronavirus
IIKISMVTGMISVISYKPRQVVEFLL

SEQ ID NO: 17 - protein, 6 aa, SARS-2 coronavirus
ILIIHC

SEQ ID NO: 18 - protein, 9 aa, SARS-2 coronavirus
LQSHMLTLT

SEQ ID NO: 19 - protein, 9 aa, SARS-2 coronavirus
QSLTLSGIC

SEQ ID NO: 20 - protein, 8 aa, SARS-2 coronavirus
NMTSRKRG

SEQ ID NO: 21 - protein, 48 aa, SARS-2 coronavirus
NSLTVILNIGIRHTTQIVLTVWMTDAFCIVQTLMFYSLQCSHLQVLDH

SEQ ID NO: 22 - protein, 10 aa, SARS-2 coronavirus
EKYLLMVFHL

SEQ ID NO: 23 - protein, 9 aa, SARS-2 coronavirus
FQLDTTSES

SEQ ID NO: 24 - protein, 7 aa, SARS-2 coronavirus
VLYIIRM

SEQ ID NO: 25 - protein, 29 aa, SARS-2 coronavirus
TYIALDLVLRNYLCMLLTLLCTLLLVIYY

SEQ ID NO: 26 - protein, 8 aa, SARS-2 coronavirus
INALRAFQ

SEQ ID NO: 27 - protein, 38 aa, SARS-2 coronavirus
LHLLTMLLFKLSNPVILTKTSMTLLCLRVSLRKEVLLN

SEQ ID NO: 28 - protein, 34 aa, SARS-2 coronavirus
NTSSLLRMVMLLSAIMTTIVIIYQQCVISDNYYL

SEQ ID NO: 29 - protein, 25 aa, SARS-2 coronavirus
LKLLISTLIVTMVAVLMLTKSSSTT

SEQ ID NO: 30 - protein, 20 aa, SARS-2 coronavirus
TNQLVFHLINGVRLDFIMIQ

SEQ ID NO: 31 - protein, 19 aa, SARS-2 coronavirus
VMRIKMHFSHIQNVMSSLL

SEQ ID NO: 32 - protein, 14 aa, SARS-2 coronavirus
ILSMPLVQRIELAP

SEQ ID NO: 33 - protein, 8 aa, SARS-2 coronavirus
LVSLSVVL

SEQ ID NO: 34 - protein, 9 aa, SARS-2 coronavirus
PIDSFIKNY

SEQ ID NO: 35 - protein, 7 aa, SARS-2 coronavirus
PPLEELL

SEQ ID NO: 36 - protein, 13 aa, SARS-2 coronavirus
LEQANSMVVGTTC

SEQ ID NO: 37 - protein, 6 aa, SARS-2 coronavirus
KLFIVM

SEQ ID NO: 38 - protein, 45 aa, SARS-2 coronavirus
KTLTLWVGIILNVIEPCLTCLELWPHLFLLANIQRVVACHTVSID

SEQ ID NO: 39 - protein, 7 aa, SARS-2 coronavirus
LMSVLKY

SEQ ID NO: 40 - protein, 78 aa, SARS-2 coronavirus
VKWSCVAVHYMLNQVEPHQEMPQLLMLIVFLTFVKLSRPMLMHFYLLMVTKLPISMSAIYNTDFMSVSIEIEMLTQTL

SEQ ID NO: 41 - protein, 12 aa, SARS-2 coronavirus
MSFTHICVNISQ

SEQ ID NO: 42 - protein, 18 aa, SARS-2 coronavirus
YSLTMLLCVSIALMHLKV

SEQ ID NO: 43 - protein, 39 aa, SARS-2 coronavirus
RTLSQFFIIKTMFLCLKQNVGLRLTLLKDLMNFALNIQC

SEQ ID NO: 44 - protein, 18 aa, SARS-2 coronavirus
LNRVMIMCTFLTQIHQES

SEQ ID NO: 45 - protein, 5 aa, SARS-2 coronavirus
GPAVL

SEQ ID NO: 46 - protein, 6 aa, SARS-2 coronavirus
KQMVHL

SEQ ID NO: 47 - protein, 6 aa, SARS-2 coronavirus
LNGSCL

SEQ ID NO: 48 - protein, 23 aa, SARS-2 coronavirus
MLTHLLNILIRSMLMSFICTYNT

SEQ ID NO: 49 - protein, 6 aa, SARS-2 coronavirus
ESYMMS

SEQ ID NO: 50 - protein, 4 aa, SARS-2 coronavirus
QDTC

SEQ ID NO: 51 - protein, 32 aa, SARS-2 coronavirus
TCILLCLLMITLQGIGNLSFMRLCTHRIQSYR

SEQ ID NO: 52 - protein, 527 aa, SARS-2 coronavirus
AENVTGLFKDCSKVITGLHPTQAPTHLSVDTKFKTEGLCVDIPGIPKDMTYRRLISMMGFKMNYQVNGYPNMFITREEAIRHVRAWIGFDVEGCHATREAVGTNLPLQLGFSTGVNLVAVPTGYVDTPNNTDFSRVSAKPPPGDQFKHLIPLMYKGLPWNVVRIKIVQMLSDTLKNLSDRVVFVLWAHGFELTSMKYFVKIGPERTCCLCDRRATCFSTASDTYACWHHSIGFDYVYNPFMIDVQQWGFTGNLQSNHDLYCQVHGNAHVASCDAIMTRCLAVHECFVKRVDWTIEYPIIGDELKINAACRKVQHMVVKAALLADKFPVLHDIGNPKAIKCVPQADVEWKFYDAQPCSDKAYKIEELFYSYATHSDKFTDGVCLFWNCNVDRYPANSIVCRFDTRVLSNLNLPGCDGGSLYVNKHAFHTPAFDKSAFVNLKQLPFFYYSDSPCESHGKQVVSDIDYVPLKSATCITRCNLGGAVCRHHANEYRLYLDAYNMMISAGFSLWVYKQFDTYNLWNTFTRLQ

SEQ ID NO: 53 - RNA, 302 nt, SARS-2 coronavirus
  misc_RNA (266)..(301): 5′ portion of ORF1a, nsp1
auuaaagguuuauaccuucccagguaacaaaccaaccaacuuucgaucucuuguagaucugcgacucuaauggucgcuuuaaaaucuguguggcugucacucggcugcaugcuuagugcacucacgcaguauaauuaauaacuaauuacugucguugacaggacacgaguaacucgucuaucuucugcaggcugcuuacgguuucguccguguugcagccgaucaucagcacaucuagguuucguccgggugugaccgaaagguaagauagagagccuugucccugguuucaacgagaaaac

SEQ ID NO: 54 - RNA, 229 nt, SARS-2 coronavirus
caaucuuuaaucaguguguaacauuagggaggacuugaaagagccaccacauuuucaccgaggccacgcggaguacgaucgaguguacagugaacaaugcuagggagagcugccuauauggaagagcccuaauguguaaaauuaauuuuaguagugcuauccccaugugauuuuaauagcuucuuaggagaaugacaaaaaaaaaaaaaaaaaaaaaaaaaaaaaaaaa

SEQ ID NO: 55-56 - skipped (2 entries)

SEQ ID NO: 57 - RNA, 544 nt, synthetic construct
  misc_feature (1)..(301): 5′ leader
  promoter (68)..(76): Replace endogenous leader TRS with eTRS to prevent recombination with native SARS-2 virus.
  part of nsp1 (267)..(301): Sequences appended to 5′ UTR to match conserved secondary structure of 5′ end of coronaviruses
  misc_feature (269)..(269)
  misc_feature (269)..(269)
  misc_feature (302)..(302): n
  promoter (306)..(314): eTRS
  misc_RNA (315)..(315): sgTranscript (subrviral transcript)
  repeat_region (315)..(315): Paired subviral promter and the transcript motif can be repeated multiple times here.
  misc_feature (315)..(315): n
  3′UTR (315)..(544)
auuaaagguuuauaccuucccagguaacaaaccaaccaacuuucgaucucuuguagaucuguucucuaauggucgcuuuaaaaucuguguggcugucacucggcugcaugcuuagugcacucacgcaguauaauuaauaacuaauuacugucguugacaggacacgaguaacucgucuaucuucugcaggcugcuuacgguuucguccguguugcagccgaucaucagcacaucuagguuucguccgggugugaccgaaagguaagauagagagccuugucccugguuucaacgagaaaacnaugaauggucgcncaaucuuuaaucaguguguaacauuagggaggacuugaaagagccaccacauuuucaccgaggccacgcggaguacgaucgaguguacagugaacaaugcuagggagagcugccuauauggaagagcccuaauguguaaaauuaauuuuaguagugcuauccccaugugauuuuaauagcuucuuaggagaaugacaaaaaaaaaaaaaaaaaaaaaaaaaaaaaaaaa

SEQ ID NO: 58 - RNA, 83 nt, synthetic construct
ccccaaaauuuccuuggguucccaaggaacagcgaaaucgaaggagaggagaggaagaggagaguauuucgcugauuuugggg

SEQ ID NO: 59 - RNA, 84 nt, synthetic construct
agccuuuuggcaauguuguuuacaacauugcguuccucacgaaggagaggagaggaagaggagagugaggaacgagaagaggcu

SEQ ID NO: 60 - RNA, 91 nt, synthetic construct
cguuuagagaacagaucuacuuguagaucuguuuugggacuauucgaaggagaggagaggaagaggagaguagucccaaaaucuuugagcg

SEQ ID NO: 61 - RNA, 122 nt, synthetic construct
acacuugcuguggaagaaauuucaaguaguucuuuucuugugcagguugggagacggucggguccagauauucguaucugucgaguagagugugggcucccaaucugcaugaauagcaagug

SEQ ID NO: 62 - RNA, 118 nt, synthetic construct
ucccagagacauguauagguucaagucuugugcauuuugguugaccgggagacggucggguccagauauucguaucugucgaguagagugugggcucccggucaauuucuuuuuggga

SEQ ID NO: 63 - RNA, 109 nt, synthetic construct
agauucauuuaaauucuugguuccaagaauuaaaaccaagggagacggucggguccagauauucguaucugucgaguagagugugggcucccuugguuuugauggaucu

SEQ ID NO: 64 - RNA, 115 nt, synthetic construct
guuuugauggaucugguaauuuuaccagauauuggaguucgggagacggucggguccagauauucguaucugucgaguagagugugggcucccguaacuccaauaccauuaaacc

SEQ ID NO: 65 - RNA, 118 nt, synthetic construct
ucugagucugauaacuagauucaaguucugguuguaagauuaacauggagacggucggguccagauauucguaucugucgaguagagugugggcuccauguuaguuucuggauugaga

SEQ ID NO: 66 - RNA, 114 nt, synthetic construct
cguuuagagaacagaucuacuuguagaucuguuuugggacuaggagacggucggguccagauauucguaucugucgaguagagugugggcuccuagucccaaaaucuuugagcg

SEQ ID NO: 67 - RNA, 134 nt, synthetic construct
gcacguacgaaggagaggagaggaagaggagaguacgugcuuauuggacaugccgaaggagaggagaggaagaggagaggcauguccuuauugggugcaucgaaggagaggagaggaagaggagagaugcaccc

SEQ ID NO: 68 - RNA, 51 nt, synthetic construct
ggagacggucggguccagauauucguaucugucgaguagagugugggcucc

SEQ ID NO: 69 - RNA, 26 nt, synthetic construct
cgaaggagaggagaggaagaggagag

SEQ ID NO: 70 - RNA, 146 nt, synthetic construct
uuucggcacguacgaaggagaggagaggaagaggagaguacgugcuuauuggacaugccgaaggagaggagaggaagaggagaggcauguccuuauugggugcaucgaaggagaggagaggaagaggagagaugcacccuuuuuaa

SEQ ID NO: 71 - RNA, 2758 nt, synthetic construct
  misc_feature (1)..(302): 5′ leader contains 5′ UTR and part of nsp1 needed to maintain conserved secondary structure.
  promoter (69)..(77): eTRS
  misc_feature (303)..(1199): mCherry proteins cds marker, nucleocapsid aptamer and SARS-2 packaging signal.
  CDS (303)..(1013): mCherry marker protein
  misc_binding (1030)..(1117): SARS-2 Nucleocapsid Aptamer
  misc_binding (1120)..(1199): Predicted SARS-2 Packaging Signal. Alignment derived from SARS-1, Qin L et al/ Acta Pharmacol Sin 2003 Jun; 24 (6) 489-496
  misc_RNA (1120)..(1199): This subviral transcript of 3 MV-RNA is tagged for Mango fluorescence with Mango aptamers embedded within each MV-RNA as well as the (3x) Mango motif.
  misc_feature (1200)..(2368): Central RNA region′s subviral transcript containing 9 MV-RNA as example.
  promoter (1200)..(1208): eTRS
  promoter (1628)..(1636): eTRS
  misc_RNA (1628)..(2370): This subviral transcript of 5 MV-RNA is tagged for DFHBI fluorescence with Broccoli aptamers embedded within each MV-RNA.
  3′UTR (2530)..(2758)
auuaaagguuuauaccuucccagguaacaaaccaaccaacuuucgaucucuuguagaucugcgacucuaauggucgcuuuaaaaucuguguggcugucacucggcugcaugcuuagugcacucacgcaguauaauuaauaacuaauuacugucguugacaggacacgaguaacucgucuaucuucugcaggcugcuuacgguuucguccguguugcagccgaucaucagcacaucuagguuucguccgggugugaccgaaagguaagauagagagccuugucccugguuucaacgagaaaacauggugagcaagggcgaggaggauaacauggccaucaucaaggaguucaugcgcuucaaggugcacauggagggcuccgugaacggccacgaguucgagaucgagggcgagggcgagggccgccccuacgagggcacccagaccgccaagcugaaggugaccaaggguggcccccugcccuucgccugggacauccuguccccucaguucauguacggcuccaaggccuacgugaagcaccccgccgacauccccgacuacuugaagcuguccuuccccgagggcuucaagugggagcgcgugaugaacuucgaggacggcggcguggugaccgugacccaggacuccucccuacaggacggcgaguucaucuacaaggugaagcugcgcggcaccaacuuccccuccgacggccccguaaugcagaagaagaccaugggcugggaggccuccuccgagcggauguaccccgaggacggcgcccugaagggcgagaucaagcagaggcugaagcugaaggacggcggccacuacgacgcugaggucaagaccaccuacaaggccaagaagcccgugcagcugcccggcgccuacaacgucaacaucaaguuggacaucaccucccacaacgaggacuacaccaucguggaacaguacgaacgcgccgagggccgccacuccaccggcggcauggacgagcuguacaaguaggggcugccgccggacggcaaugguacgguacuuccggaugcggaaacuggcuaauuggugaggcuggggcggucgugcagcaaaagugcacgcuacuuugcuaacuaaaacuauuucauaacagaugcgcaaacagguucaucuaaguguguguguucuguuauugauuuauuacuugaugauuuuaauggucgcccuaggaccccaaaauuuccuuggguucccaaggaacagcgaaaucgaaggagaggagaggaagaggagaguauuucgcugauuuugggguuucagccuuuuggcaauguuguuuacaacauugcguuccucacgaaggagaggagaggaagaggagagugaggaacgagaagaggcuuuuccguuuagagaacagaucuacuuguagaucuguuuugggacuauucgaaggagaggagaggaagaggagaguagucccaaaaucuuugagcguuucggcacguacgaaggagaggagaggaagaggagaguacgugcuuauuggacaugccgaaggagaggagaggaagaggagaggcauguccuuauugggugcaucgaaggagaggagaggaagaggagagaugcacccuuuuuaaaauggucgcaaacuaaaaacacuugcuguggaagaaauuucaaguaguucuuuucuugugcagguugggagacggucggguccagauauucguaucugucgaguagagugugggcucccaaucugcaugaauagcaaguguuucucccagagacauguauagguucaagucuugugcauuuugguugaccgggagacggucggguccagauauucguaucugucgaguagagugugggcucccggucaauuucuuuuugggauuucagauucauuuaaauucuugguuccaagaauuaaaaccaagggagacggucggguccagauauucguaucugucgaguagagugugggcucccuugguuuugauggaucuuuucguuuugauggaucugguaauuuuaccagauauuggaguucgggagacggucggguccagauauucguaucugucgaguagagugugggcucccguaacuccaauaccauuaaaccuuucucugagucugauaacuagauucaaguucugguuguaagauuaacauggagacggucggguccagauauucguaucugucgaguagagugugggcuccauguuaguuucuggauugagauuuccguuuagagaacagaucuacuuguagaucuguuuugggacuaggagacggucggguccagauauucguaucugucgaguagagugugggcuccuagucccaaaaucuuugagcguugguaccuaaacucaugcagaccacacaaggcagaugggcuauauaaacguuuucgcuuuuccguuuacgauauauagucuacucuugugcagaaugaauucucguaacuacauagcacaaguagauguaguuaacuuuaaucucacauagcguccggcggcagccccaaucuuuaaucaguguguaacauuagggaggacuugaaagagccaccacauuuucaccgaggccacgcggaguacgaucgaguguacagugaacaaugcuagggagagcugccuauauggaagagcccuaauguguaaaauuaauuuuaguagugcuauccccaugugauuuuaauagcuucuuaggagaaugacaaaaaaaaaaaaaaaaaaaaaaaaaaaaaaaaa

SEQ ID NO: 72 - protein, 236 aa, synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 73 - RNA, 10735 nt, Dengue virus
  5′UTR (1)..(94)
  misc_feature (132)..(142): Conserved 5′ Cyclization motif
  CDS (7574)..(10270): RdRp
  3′UTR (10275)..(10735)
  misc_feature (10630)..(10640): Conserved 3′ Cyclization motif
aguuguuagucuacguggaccgacaagaacaguuucgaaucggaagcuugcuuaacguaguucuaacaguuuuuuauuagagagcagaucucugaugaacaaccaacggaaaaagacgggucgaccgucuuucaauaugcugaaacgcgcgagaaaccgcgugucaacuguuucacaguuggcgaagagauucucaaaaggauugcuuucaggccaaggacccaugaaauuggugauggcuuuuauagcauuccuaagauuucuagccauaccuccaacagcaggaauuuuggcuagauggggcucauucaagaagaauggagcgaucaaaguguuacgggguuucaagaaagaaaucucaaacauguugaacauaaugaacaggaggaaaagaucugugaccaugcuccucaugcugcugcccacagcccuggcguuccaucugaccacccgagggggagagccgcacaugauaguuagcaagcaggaaagaggaaaaucacuuuuguuuaagaccucugcaggugucaacaugugcacccuuauugcaauggauuugggagaguuaugugaggacacaaugaccuacaaaugcccccggaucacugagacggaaccagaugacguugacuguuggugcaaugccacggagacaugggugaccuauggaacauguucucaaacuggugaacaccgacgagacaaacguuccgucgcacuggcaccacacguagggcuuggucuagaaacaagaaccgaaacguggauguccucugaaggcgcuuggaaacaaauacaaaaaguggagaccugggcucugagacacccaggauucacggugauagcccuuuuucuagcacaugccauaggaacauccaucacccagaaagggaucauuuuuauuuugcugaugcugguaacuccauccauggccaugcggugcgugggaauaggcaacagagacuucguggaaggacugucaggagcuacguggguggaugugguacuggagcauggaaguugcgucacuaccauggcaaaagacaaaccaacacuggacauugaacucuugaagacggaggucacaaacccugccguccugcgcaaacugugcauugaagcuaaaauaucaaacaccaccaccgauucgagauguccaacacaaggagaagccacgcugguggaagaacaggacacgaacuuugugugucgacgaacguucguggacagaggcuggggcaaugguugugggcuauucggaaaagguagcuuaauaacgugugcuaaguuuaagugugugacaaaacuggaaggaaagauaguccaauaugaaaacuuaaaauauucagugauagucaccguacacacuggagaccagcaccaaguuggaaaugagaccacagaacauggaacaacugcaaccauaacaccucaagcucccacgucggaaauacagcugacagacuacggagcucuaacauuggauuguucaccuagaacagggcuagacuuuaaugagaugguguuguugacaaugaaaaaaaaaucauggcucguccacaaacaaugguuucuagacuuaccacugccuuggaccucgggggcuucaacaucccaagagacuuggaauagacaagacuugcuggucacauuuaagacagcucaugcaaaaaagcaggaaguagucguacuaggaucacaagaaggagcaaugcacacugcguugacuggagcgacagaaauccaaacgucuggaacgacaacaauuuuugcaggacaccugaaaugcagauuaaaaauggauaaacugauuuuaaaagggaugucauauguaaugugcacagggucauucaaguuagagaaggaaguggcugagacccagcauggaacuguucuagugcagguuaaauacgaaggaacagaugcaccaugcaagauccccuucucgucccaagaugagaagggaguaacccagaaugggagauugauaacagccaaccccauagucacugacaaagaaaaaccagucaacauugaagcggagccaccuuuuggugagagcuacauugugguaggagcaggugaaaaagcuuugaaacuaagcugguucaagaagggaagcaguauagggaaaauguuugaagcaacugcccguggagcacgaaggauggccauccugggagacacugcaugggacuucgguucuauaggagggguguucacgucugugggaaaacugauacaccagauuuuugggacugcguauggaguuuuguucagcgguguuucuuggaccaugaagauaggaauagggauucugcugacauggcuaggauuaaacucaaggagcacgucccuuucaaugacguguaucgcaguuggcauggucacacuguaccuaggagucaugguucaggcggacucgggauguguaaucaacuggaaaggcagagaacucaaauguggaagcggcauuuuugucaccaaugaaguccacaccuggacagagcaauauaaauuccaggccgacuccccuaagagacuaucagcggccauugggaaggcaugggaggaggguguguguggaauucgaucagccacucgucucgagaacaucauguggaagcaaauaucaaaugaauuaaaccacaucuuacuugaaaaugacaugaaauuuacaguggucguaggagacguuaguggaaucuuggcccaaggaaagaaaaugauuaggccacaacccauggaacacaaauacucguggaaaagcuggggaaaagccaaaaucauaggagcagauguacagaauaccaccuucaucaucgacggcccaaacaccccagaaugcccugauaaccaaagagcauggaacauuugggaaguugaagacuauggauuuggaauuuucacgacaaacauaugguugaaauugcgugacuccuacacucaagugugugaccaccggcuaaugucagcugccaucaaggauagcaaagcaguccaugcugacaugggguacuggauagaaagugaaaagaacgagacuuggaaguuggcaagagccuccuucauagaaguuaagacaugcaucuggccaaaaucccacacucuauggagcaauggaguccuggaaagugagaugauaaucccaaagauauauggaggaccaauaucucagcacaacuacagaccaggauauuucacacaaacagcagggccguggcacuugggcaaguuagaacuagauuuugauuuaugugaagguaccacuguuguuguggaugaacauuguggaaaucgaggaccaucucuuagaaccacaacagucacaggaaagacaauccaugaauggugcuguagaucuugcacguuacccccccuacguuucaaaggagaagacgggugcugguacggcauggaaaucagaccagucaaggagaaggaagagaaccuaguuaagucaauggucucugcagggucaggagaaguggacaguuuuucacuaggacugcuaugcauaucaauaaugaucgaagagguaaugagauccagauggagcagaaaaaugcugaugacuggaacauuggcuguguuccuccuucucacaaugggacaauugacauggaaugaucugaucaggcuauguaucaugguuggagccaacgcuucagacaagauggggaugggaacaacguaccuagcuuugauggccacuuucagaaugagaccaauguucgcagucgggcuacuguuucgcagauuaacaucuagagaaguucuucuucuuacaguuggauugagucugguggcaucuguagaacuaccaaauuccuuagaggagcuaggggauggacuugcaaugggcaucaugauguugaaauuacugacugauuuucagucacaucagcuaugggcuaccuugcugucuuuaacauuugucaaaacaacuuuuucauugcacuaugcauggaagacaauggcuaugauacugucaauuguaucucucuucccuuuaugccuguccacgacuucucaaaaaacaacauggcuuccgguguugcugggaucucuuggaugcaaaccacuaaccauguuucuuauaacagaaaacaaaaucuggggaaggaaaagcuggccucucaaugaaggaauuauggcuguuggaauaguuagcauucuucuaaguucacuucucaagaaugaugugccacuagcuggcccacuaauagcuggaggcaugcuaauagcauguuaugucauaucuggaagcucggccgauuuaucacuggagaaagcggcugaggucuccugggaagaagaagcagaacacucuggugccucacacaacauacuaguggagguccaagaugauggaaccaugaagauaaaggaugaagagagagaugacacacucaccauucuccucaaagcaacucugcuagcaaucucagggguauacccaaugucaauaccggcgacccucuuugugugguauuuuuggcagaaaaagaaacagagaucaggagugcuaugggacacacccagcccuccagaaguggaaagagcaguccuugaugauggcauuuauagaauucuccaaagaggauuguugggcaggucucaaguaggaguaggaguuuuucaagaaggcguguuccacacaauguggcacgucaccaggggagcuguccucauguaccaagggaagagacuggaaccaaguugggccagugucaaaaaagacuugaucucauauggaggagguuggagguuucaaggauccuggaacgcgggagaagaagugcaggugauugcuguugaaccggggaagaaccccaaaaauguacagacagcgccggguaccuucaagaccccugaaggcgaaguuggagccauagcucuagacuuuaaacccggcacaucuggaucuccuaucgugaacagagagggaaaaauaguaggucuuuauggaaauggaguggugacaacaagugguaccuacgucagugccauagcucaagcuaaagcaucacaagaagggccucuaccagagauugaggacgagguguuuaggaaaagaaacuuaacaauaauggaccuacauccaggaucgggaaaaacaagaagauaccuuccagccauaguccgugaggccauaaaaagaaagcugcgcacgcuagucuuagcucccacaagaguugucgcuucugaaauggcagaggcgcucaagggaaugccaauaagguaucagacaacagcagugaagagugaacacacgggaaaggagauaguugaccuuaugugucacgccacuuucacuaugcgucuccugucuccugugagaguucccaauuauaauaugauuaucauggaugaagcacauuuuaccgauccagccagcauagcagccagaggguauaucucaacccgaguggguaugggugaagcagcugcgauuuucaugacagccacuccccccggaucgguggaggccuuuccacagagcaaugcaguuauccaagaugaggaaagagacauuccugaaagaucauggaacucaggcuaugacuggaucacugauuucccagguaaaacagucugguuuguuccaagcaucaaaucaggaaaugacauugccaacuguuuaagaaagaaugggaaacgggugguccaauugagcagaaaaacuuuugacacugaguaccagaaaacaaaaaauaacgacugggacuauguugucacaacagacauauccgaaaugggagcaaacuuccgagccgacaggguaauagacccgaggcggugccugaaaccgguaauacuaaaagauggcccagagcgugucauucuagccggaccgaugccagugacuguggcuagcgccgcccagaggagaggaagaauuggaaggaaccaaaauaaggaaggcgaucaguauauuuacaugggacagccucuaaacaaugaugaggaccacgcccauuggacagaagcaaaaaugcuccuugacaacauaaacacaccagaagggauuaucccagcccucuuugagccggagagagaaaagagugcagcaauagacggggaauacagacuacggggugaagcgaggaaaacguucguggagcucaugagaagaggagaucuaccugucuggcuauccuacaaaguugccucagaaggcuuccaguacuccgacagaagguggugcuuugauggggaaaggaacaaccagguguuggaggagaacauggacguggagaucuggacaaaagaaggagaaagaaagaaacuacgaccccgcuggcuggaugccagaacauacucugacccacuggcucugcgcgaauucaaagaguucgcagcaggaagaagaagcgucucaggugaccuaauauuagaaauagggaaacuuccacaacauuuaacgcaaagggcccagaacgccuuggacaaucugguuauguugcacaacucugaacaaggaggaaaagccuauagacacgccauggaagaacuaccagacaccauagaaacguuaaugcuccuagcuuugauagcugugcugacugguggagugacguuguucuuccuaucaggaaggggucuaggaaaaacauccauuggccuacucugcgugauugccucaagugcacuguuauggauggccaguguggaaccccauuggauagcggccucuaucauacuggaguucuuucugaugguguugcuuauuccagagccggacagacagcgcacuccacaagacaaccagcuagcauacguggugauaggucuguuauucaugauauugacaguggcagccaaugagaugggauuacuggaaaccacaaagaaggaccuggggauuggucaugcagcugcugaaaaccaccaucaugcugcaaugcuggacguagaccuacauccagcuucagccuggacucucuaugcaguggccacaacaauuaucacucccaugaugagacacacaauugaaaacacaacggcaaauauuucccugacagcuauugcaaaccaggcagcuauauugaugggacuugacaagggauggccaauaucaaagauggacauaggaguuccacuucucgccuuggggugcuauucucaggugaacccgcugacgcugacagcggcgguauugaugcuaguggcucauuaugccauaauuggacccggacugcaagcaaaagcuacuagagaagcucaaaaaaggacagcagccggaauaaugaaaaacccaacugucgacgggaucguugcaauagauuuggacccugugguuuacgaugcaaaauuugaaaaacagcuaggccaaauaauguuguugauacuuugcacaucacagauccuccugaugcggaccacaugggccuugugugaauccaucacacuagccacuggaccucugacuacgcuuugggagggaucuccaggaaaauucuggaacaccacgauagcgguguccauggcaaacauuuuuaggggaaguuaucuagcaggagcaggucuggccuuuucauuaaugaaaucucuaggaggagguaggagaggcacgggagcccaaggggaaacacugggagaaaaauggaaaagacagcuaaaccaauugagcaagucagaauucaacacuuacaaaaggagugggauuauagagguggauagaucugaagccaaagagggguuaaaaagaggagaaacgacuaaacacgcagugucgagaggaacggccaaacugaggugguuuguggagaggaaccuugugaaaccagaagggaaagucauagaccucgguuguggaagagguggcuggucauauuauugcgcugggcugaagaaagucacagaagugaaaggauacacgaaaggaggaccuggacaugaggaaccaaucccaauggcaaccuauggauggaaccuaguaaagcuauacuccgggaaagauguauucuuuacaccaccugagaaaugugacacccucuugugugauauuggugaguccucuccgaacccaacuauagaagaaggaagaacguuacguguucuaaagaugguggaaccauggcucagaggaaaccaauuuugcauaaaaauucuaaaucccuauaugccgagugugguagaaacuuuggagcaaaugcaaagaaaacauggaggaaugcuagugcgaaauccacucucaagaaacuccacucaugaaauguacuggguuucauguggaacaggaaacauugugucagcaguaaacaugacaucuagaaugcugcuaaaucgauucacaauggcucacaggaagccaacauaugaaagagacguggacuuaggcgcuggaacaagacauguggcaguagaaccagagguggccaaccuagauaucauuggccagaggauagagaauauaaaaaaugaacacaaaucaacauggcauuaugaugaggacaauccauacaaaacaugggccuaucauggaucauaugaggucaagccaucaggaucagccucauccauggucaaugguguggugagacugcuaaccaaaccaugggaugucauucccauggucacacaaauagccaugacugacaccacacccuuuggacaacagaggguguuuaaagagaaaguugacacgcguacaccaaaagcgaaacgaggcacagcacaaauuauggaggugacagccaggugguuaugggguuuucucucuagaaacaaaaaacccagaaucugcacaagagaggaguucacaagaaaagucaggucaaacgcagcuauuggagcaguguucguugaugaaaaucaauggaacucagcaaaagaggcaguggaagaugaacgguucugggaccuugugcacagagagagggagcuucauaaacaaggaaaaugugccacgugugucuacaacaugaugggaaagagagagaaaaaauuaggagaguucggaaaggcaaaaggaagucgcgcaauaugguacaugugguugggagcgcgcuuuuuagaguuugaagcccuugguuucaugaaugaagaucacugguucagcagagagaauucacucaguggaguggaaggagaaggacuccacaaacuuggauacauacucagagacauaucaaagauuccagggggaaauauguaugcagaugacacagccggaugggacacaagaauaacagaggaugaucuucagaaugaggccaaaaucacugacaucauggaaccugaacaugcccuauuggccacgucaaucuuuaagcuaaccuaccaaaacaagguaguaagggugcagagaccagcgaaaaauggaaccgugauggaugucauauccagacgugaccagagaggaaguggacagguuggaaccuauggcuuaaacaccuucaccaacauggaggcccaacuaauaagacaaauggagucugagggaaucuuuucacccagcgaauuggaaaccccaaaucuagccgaaagaguccucgacugguugaaaaaacauggcaccgagaggcugaaaagaauggcaaucaguggagaugacuguguggugaaaccaaucgaugacagauuugcaacagccuuaacagcuuugaaugacaugggaaagguaagaaaagacauaccgcaaugggaaccuucaaaaggauggaaugauuggcaacaagugccuuucuguucacaccauuuccaccagcugauuaugaaggaugggagggagauaguggugccaugccgcaaccaagaugaacuuguagguagggccagaguaucacaaggcgccggauggagcuugagagaaacugcaugccuaggcaagucauaugcacaaauguggcagcugauguacuuccacaggagagacuugagauuagcggcuaaugcuaucuguucagccguuccaguugauugggucccaaccagccgcaccaccuggucgauccaugcccaccaucaauggaugacaacagaagacauguugucaguguggaauaggguuuggauagaggaaaacccauggauggaggacaagacucauguguccaguugggaagacguuccauaccuaggaaaaagggaagaucaauggugugguucccuaauaggcuuaacagcacgagccaccugggccaccaacauacaaguggccauaaaccaagugagaaggcucauugggaaugagaauuaucuagacuucaugacaucaaugaagagauucaaaaacgagagugaucccgaaggggcacucugguaagccaacucauucacaaaauaaaggaaaauaaaaaaucaaacaaggcaagaagucaggccggauuaagccauagcacgguaagagcuaugcugccugugagccccguccaaggacguaaaaugaagucaggccgaaagccacgguucgagcaagccgugcugccuguagcuccaucguggggauguaaaaacccgggaggcugcaaaccauggaagcuguacgcaugggguagcagacuagugguuagaggagaccccucccaagacacaacgcagcagcggggcccaacaccaggggaagcuguacccuggugguaaggacuagagguuagaggagaccccccgcacaacaacaaacagcauauugacgcugggagagaccagagauccugcugucucuacagcaucauuccaggcacagaacgccaaaaaauggaauggugcuguugaaucaacagguucu

SEQ ID NO: 74 - protein, 899 aa, Dengue virus
GTGAQGETLGEKWKRQLNQLSKSEFNTYKRSGIIEVDRSEAKEGLKRGETTKHAVSRGTAKLRWFVERNLVKPEGKVIDLGCGRGGWSYYCAGLKKVTEVKGYTKGGPGHEEPIPMATYGWNLVKLYSGKDVFFTPPEKCDTLLCDIGESSPNPTIEEGRTLRVLKMVEPWLRGNQFCIKILNPYMPSVVETLEQMQRKHGGMLVRNPLSRNSTHEMYWVSCGTGNIVSAVNMTSRMLLNRFTMAHRKPTYERDVDLGAGTRHVAVEPEVANLDIIGQRIENIKNEHKSTWHYDEDNPYKTWAYHGSYEVKPSGSASSMVNGVVRLLTKPWDVIPMVTQIAMTDTTPFGQQRVFKEKVDTRTPKAKRGTAQIMEVTARWLWGFLSRNKKPRICTREEFTRKVRSNAAIGAVFVDENQWNSAKEAVEDERFWDLVHRERELHKQGKCATCVYNMMGKREKKLGEFGKAKGSRAIWYMWLGARFLEFEALGFMNEDHWFSRENSLSGVEGEGLHKLGYILRDISKIPGGNMYADDTAGWDTRITEDDLQNEAKITDIMEPEHALLATSIFKLTYQNKVVRVQRPAKNGTVMDVISRRDQRGSGQVGTYGLNTFTNMEAQLIRQMESEGIFSPSELETPNLAERVLDWLKKHGTERLKRMAISGDDCVVKPIDDRFATALTALNDMGKVRKDIPQWEPSKGWNDWQQVPFCSHHFHQLIMKDGREIVVPCRNQDELVGRARVSQGAGWSLRETACLGKSYAQMWQLMYFHRRDLRLAANAICSAVPVDWVPTSRTTWSIHAHHQWMTTEDMLSVWNRVWIEENPWMEDKTHVSSWEDVPYLGKREDQWCGSLIGLTARATWATNIQVAINQVRRLIGNENYLDFMTSMKRFKNESDPEGALW

SEQ ID NO: 75 - RNA, 147 nt, Dengue virus
  promoter (132)..(142): Conserved Sequence (CS), aka 5′ cyclization sequence
aguuguuagucuacguggaccgacaagaacaguuucgaaucggaagcuugcuuaacguaguucuaacaguuuuuuauuagagagcagaucucugaugaacaaccaacggaaaaagacgggucgaccgucuuucaauaugcugaaacg

SEQ ID NO: 76 - RNA, 461 nt, Dengue virus
  promoter (355)..(365): Conserved Sequence (CS), aka 3′ cyclization sequence
ccaacucauucacaaaauaaaggaaaauaaaaaaucaaacaaggcaagaagucaggccggauuaagccauagcacgguaagagcuaugcugccugugagccccguccaaggacguaaaaugaagucaggccgaaagccacgguucgagcaagccgugcugccuguagcuccaucguggggauguaaaaacccgggaggcugcaaaccauggaagcuguacgcaugggguagcagacuagugguuagaggagaccccucccaagacacaacgcagcagcggggcccaacaccaggggaagcuguacccuggugguaaggacuagagguuagaggagaccccccgcacaacaacaaacagcauauugacgcugggagagaccagagauccugcugucucuacagcaucauuccaggcacagaacgccaaaaaauggaauggugcuguugaaucaacagguucu

SEQ ID NO: 77 - RNA, 11 nt, Dengue virus
ucaauaugcug

SEQ ID NO: 78 - RNA, 11 nt, Dengue virus
cagcauauuga

SEQ ID NO: 79-80 - skipped (2 entries)

SEQ ID NO: 81 - RNA, 87 nt, synthetic construct
guguuacacuuggcguucgcgaaggagaggagaggaagaggagagcgaacgccaucaguaaauuuauuuacugaaaauguaacauuu

SEQ ID NO: 82 - RNA, 88 nt, synthetic construct
gcugaugguguucuucgcgucgaaggagaggagaggaagaggagagacgcgaagacucguuacauuuguaacgaguuuccgucagcuu

SEQ ID NO: 83 - RNA, 85 nt, synthetic construct
acaguuugguguccugcugcgaaggagaggagaggaagaggagagcagcaggacggccaucauuuaugauggccucgaacuguuu

SEQ ID NO: 84 - RNA, 88 nt, synthetic construct
gaauuuuccuuuguucggcccgaaggagaggagaggaagaggagagggccgaacaguugugguuuuaaccacaaccaggaaaauucuu

SEQ ID NO: 85 - RNA, 84 nt, synthetic construct
ugaucgucuuccagaucucgaaggagaggagaggaagaggagagagaucuggaagaacaccuuuagguguucugggcgaucauu

SEQ ID NO: 86 - RNA, 449 nt, synthetic construct
  misc_RNA (1)..(449)
guguuacacuuggcguucgcgaaggagaggagaggaagaggagagcgaacgccaucaguaaauuuauuuacugaaaauguaacauuucuuuugcugaugguguucuucgcgucgaaggagaggagaggaagaggagagacgcgaagacucguuacauuuguaacgaguuuccgucagcuuuuacaguuugguguccugcugcgaaggagaggagaggaagaggagagcagcaggacggccaucaucuuuuaugauggccucgaacuguuuuugaauuuuccuuuguucggcccgaaggagaggagaggaagaggagagggccgaacaguugugguucuuuuaaccacaaccaggaaaauucuuuuugaucgucuuccagaucucgaaggagaggagaggaagaggagagagaucuggaagaacaccuuuagguguucugggcgaucauu

SEQ ID NO: 87 - RNA, 1052 nt, synthetic construct
  5′UTR (1)..(94)
  misc_RNA (1)..(140): 5′ leader
  promoter (132)..(140): Engineered 5′ cyclization motif
  misc_RNA (144)..(592): Contains and example series of MV-RNA
  3′UTR (594)..(1052)
  promoter (949)..(957): Engineered 3′ cyclization motif
aguuguuagucuacguggaccgacaagaacaguuucgaaucggaagcuugcuuaacguaguucuaacaguuuuuuauuagagagcagaucucugaugaacaaccaacggaaaaagacgggucgaccgucuuaauggucgcugaguguuacacuuggcguucgcgaaggagaggagaggaagaggagagcgaacgccaucaguaaauuuauuuacugaaaauguaacauuucuuuugcugaugguguucuucgcgucgaaggagaggagaggaagaggagagacgcgaagacucguuacauuuguaacgaguuuccgucagcuuuuacaguuugguguccugcugcgaaggagaggagaggaagaggagagcagcaggacggccaucaucuuuuaugauggccucgaacuguuuuugaauuuuccuuuguucggcccgaaggagaggagaggaagaggagagggccgaacaguugugguucuuuuaaccacaaccaggaaaauucuuuuugaucgucuuccagaucucgaaggagaggagaggaagaggagagagaucuggaagaacaccuuuagguguucugggcgaucauugccaacucauucacaaaauaaaggaaaauaaaaaaucaaacaaggcaagaagucaggccggauuaagccauagcacgguaagagcuaugcugccugugagccccguccaaggacguaaaaugaagucaggccgaaagccacgguucgagcaagccgugcugccuguagcuccaucguggggauguaaaaacccgggaggcugcaaaccauggaagcuguacgcaugggguagcagacuagugguuagaggagaccccucccaagacacaacgcagcagcggggcccaacaccaggggaagcuguacccuggugguaaggacuagagguuagaggagaccccccgcacaacaacaaagcgaccauucgcugggagagaccagagauccugcugucucuacagcaucauuccaggcacagaacgccaaaaaauggaauggugcuguugaaucaacagguucu

SEQ ID NO: 88 - RNA, 13202 nt, Dicistrovirus DVVv3
  5′ UTR (1)..(675)
  CDS (7420)..(9066): ORF1 protein, RdRp
  promoter (9193)..(9234): IGR intergenic region
  CDS (9235)..(12456): ORF2, structural protein. Replaced with a Central Region of tandem MV-RNA as in FIG. 1.
  3′ UTR (12454)..(13202)
ccauuacggccgggggaaaauaagggcuauggcgucaugccauaguuuauauacaccacggauuacgugaaccuuguuaguaauaacgggugcccuuuguuagggauguguuuuaugaauuauuaauuauguuauaacucuuauuuucaacuacgggauuaaaaccccacuuuaaaacuauauagggaaauuuaguuuaaaccuguauaggaaauuaaggauugauaauuuuauugaacgagccuacucguugaauauuuauaguuaguugugauaacuagccuuguauuuagacauggauaaucuccagcccauguucgacauaccuuaggaaguuuauaugcggcacguauucaauuaaaugaagaugcuauaagugcuugauuagacauuaaaacuagaacaaguucucuaguugucgcugguagagcauaacuuauaguagguggauuuuauuugaugacccaaguauuuaugaaauuugucggccuccaaguugaucagcuaauuauuuauauuuugcgaguaauaguaguucagacugugcuauccccuugacaugcauuuuuugguuacuccagauugugauauuagggcucuaauauugcaagacugcccaccaaguaaggaacuuauguaaacuugcuugggaaacacauuacucaaaaaauuauuauggaaaguucaaauaagaauauguguacuggauucccccaaacccuuaaaaucucaacggaaauuaguaucauaaaccaaucaucucaaacccccagagauuggucuuauguacaagaaaauaugcaacaguuuaaaaauuauuuacaauuagcauggauugaaguuccgaauaacauuacaauaggaaauuuugacguuuuugaagauaauaugccaauugcucacgcuauuucaucggauuugcaucaaacagcagguauugcugguguuuuaagacaaaauuauuuuaaaaaucuaccuaaaguuaacguaaauaaaaguaauauuggaaccuuauauuuguauccaaauaaaaauuuaacucgacaaauuauuggauugauuacuaaagauauuuauuauuuaaaaccaacuuaugcuucacuuuuuacaacuuuaauugaauugcguaaauaucuuauuaauaauaaaauaaaaaauauuugccuaccuaugguagguuguaauaaagauaaauuaaauccucuugcaguuuuacccaugcuaauguauugccucggcaauauugaaacucaaguaaaaauaacuaauaaaggaguuuauuuuaauuaucaaccaauuccuguugcuccaaaaauuaauaaaauuaaauuuaguaaacaaauggaaaaaguuaauuaugaucaaacuguagauacuuuugaaacaaauuauguuaaaccuaagaaauucaaagcuaaaguaacauuugaacccucucuuuuaaaucuugauucuaauaguauuauaauuaaugaaaguaagacaguaaaacaaauuuauaaagcuaaauuagaaaaaguaauauaucaaagaucuacaguuuaugaaccacaaaagaagacauaugugguagaagcuuuuucuuguaaacaaccuaaaccaauugucaaaauuuuuaaaaugaaacaagcaauuuuaacucaaacugacuuaaaagaaaauaauaaugaacuaauuaaagaaagaaauaguauugauuauuauaaauuuguuacaccuaacaccguuaaagcuauuaucggauacgacaaaguagaaucaguuuugacauuugauuuggaucguaaacguagauguggagaaaaagcuuuuguacauggaaaaucuuuaaaaucaauagcuaaacgacaacuguguuauaguuauguaccaucucuaaacucagugcaaguauauauuaaaauguuacaauuaacggcgcagaaacaaguauuuggucauguaguaacgaaaauucaagaaaguauugccaauauggauguucauggaaugcaaauuaacaauauaccugaagaauuaaugaaaucauuauccagaacaauuuuagaacaauuacaauuagacacuaaaguaauaaaaccaguuuuguuagcaaauauauuaaauuguuucguaaauguuguuugugccuuaauaauaauauucacugccaaaaguaaaguugcaucuggagcagcuauuguucaaauaauuucaaauauuuuaaccuuaauguuggauauaggcauuuauuauucacuuaaacauaugaccgaagaaagaauuaaagcaauaacuacgacuauaacacaggaugcuguccaaaaaguaaugaaacaacauggcguaacuaugauggcaucugauaaaguaauaacagccaugcaaaaugucgauuuaaaugaugcuaaaucaauaaagcauuuuauaaaagaauucgaaaaagaagaagauaccgacauuucagaaauuucacccgauuguguaccaaacuauucugauauguuaccuauuggauuacaaaugaaaaagaagaacaggcuugauauggauuuuguuuugauggacaaaauagcaguuguugauccaacggguaaguuaaugaagauaguuagugacuguccauacgacacaccuaguaaaaauaaucaguguuugcuccacucccuucugggugcacuagaucauagagauagaaaaguuuuaguggaugaaaguaauauugaaaaauucuuccaauggauguccgauaauauuagugauccagaugugauugaagcaauuaauguuuauaaaauggaguaugcuucugauuucccaccagaagcaacuaauuuagauguuuuacaaggaauuaaaacucauaagaaaauguuaccaguugaccuuuguaaauuacucucaaaauauuuggaccguacuauaauuguaaaacauguaaauaaaguuuugacuuauggaccggaaagaugggauaacaauauaccaaaaauacuccaauuagaaaaucaacauuuuagcuauauugaccgaguacuaguggauaauauuuugccauuaaaagcagcaguugaguguaugguucaauuuacuuuaacacgagccgcacaauuuaauacacccgauacacuuaaacaaaaagaguauuauuuagcgauggcagcagcauacguuuuuacauauaauucuagaauugcuguguguuguaugaaugugaaaucuaauaagcuugaugcgaaaucaauuauugguauuugcacagauauucugucuacaauugcuuucguaguugguaugggaguaucuaguggugauuuugcaucacgcaugguaaaggcugagaaauuuucaacaucuauggauacauuugcaucuaggacaacuaauaaguuagauucgauuuuaucgaaagcuuucgguauuuauaugucggaagauggcgaaacgguugcaaguuuauccaaagauauugauauacuagagaaauauaaagcacauacuguagccgauuauaugaauaauaugucaaaauaucaagaaguuaaaacauauguagaaggaguuacaaaacguaugacaucacuacaccuauuaaauagagauaccacuaaugcuauucaaaauacgcaaaauauuauaagaaagaguugugaugccauaauggcaguuuugaaugacguugauaaguugaauagaauggaaucauugagacaggaauuugugggccuuaagggagcugguaaaaccucaacauugaacaauuaucuaauaccaaauauugcuaagcgcuuggcguugaauccaagcaccuaugcagcagaaauugacgcgaauggacauuggaaacccuauagauaugaagauuuugcuuuaauagaugauuuguuuaaucaggguaguaacgaccccuuuauaccacauauuguaaggauggcuuccucucuuaacuuugauuuaccuggagcugacuugagcaauaaagagaaaacuuuaagggcuagauucuuguuugcgacagcaaacguugcauauguaagaaugaaggaagaaaugcccgcuagugcucaggaaggauuuuaugguagauuuaaguguauucaauuuauuaaugccaaacaagguauuaaucaggcaagagauucaguggaauacaucgacuaugaagaucaugauacauaugaauugagaauguaugaagaacaaauuaaugaacaauucucacagauugaaagaaaugaccauauaacuggugaacguauacaacauuuugauccuucuacuaagacuuaugaugaccauauuuacaaaauaggacauuuacauuauaaaauuguaaccuuuaaacaguuagaaacaauuuguauagaucaguuuaauaaaaaguaucaacauuacaaaauggcuagagaucguaaauguguaguggacuuugaauauuuuagaaauauugauaugaaaucagacgcaauaguagaaaauaugguacguaaggguuaucucccagcagaaauugaagcagcaagacaaacuguaggacuuacagcuggaccagaugaagugcaugccaaaucugaaucuauaauuaaucauuacguuguaucaguuuuuggcgauacuggaacuggaaaaucuguuuuugcuacacagcuggcguuagaucugucagacuuucugaauuugaaaguauauucuguggaagauauaaaugauacggcccauuuagacuacuuaaauccaaguauaauuguuuuccaugauagcauuuaugaugaauugaaauauguugcauuauaugauaaauuaccaaaaccauguuuaauaauaacuacaaguaaucauacauuaaaauggcauagagagaaauuauguaauauuguugauaauaguacuucagcagcagagcgcucuucuagugcuuuguuggaacaugguuucuuuaacaaaaugaaaaauaaaauuggcggacuuguuucuuauuuuaauuugucaaguauuagagaagguuauuugguucaaacauucccaggauaccaaccaacaccugguuauuguagaagacuagguuugaacacugcauuccacccgauaucgacagaagauuugacaucuaauuuaguauauucacagcguccuaauaaagaaacuacaaccauuuuugcaacuucaggauaccauuuccaucaggauaauaaagaagugcaauuaaacgaagucguuaaagaaguuuuaaugaaauauagaggagcuaagaaauucauucagccugauauuaucaaauuaggaguaucagaauuugaggaacaauuuaagaaauuaacaacuauugauguagaaauucagucuaaaaauauugagucauugauuaaauuaacgaacgauagagcuauacuaguaaacucuguaacugcagcuagauuuguuggugguucuaauauuguuaaaguuuccaguagagucagaucaaauaauuauucuuuuaauccucuagauuuugcuauuccuauuaaugcuacuguaucugaugcacuagauauagcaaugaaaucuuauaggcuauuauugagugcaaauguagauuauacaguauuuaucaaaugugaagaauuuucuuguauagguauuaacggcaauauuuacuauacuucaguaccuuuuaauguaacuucuuauucacauauugcaacuaaaguaauaacauuuuauaaaguagauacuaauucagauggaauaacaaauaauucagaauuaaucaaauuuacaccggaugaauaugcuaaauuaauuauuacaggagaaggagaacaaaaacuuuuugauaaguugccuuaugcagcuuguaaauauauuuauggcgcuagagcagagauugaaccacauuuugcgacaacauuuacuaaauacgaaguuagaacagaaguuuaugauaaucauacuagagagaaaauggcuuucuuuguaauuuauucagaauuuaagaaaacaauuugguauaaaauagcuauagguauuuuggcuauaacugcaucaguuuuaauaauugaauuaauaacacaaauuuggaauauguuuauugguuuaaaagaaguuuuuacaaguaccguucaaaaagauaauguuaaaguaguuuaucaaguuuuaucaguuaauccuaaagagcaaucagcagaaguuaaaguuuaugguuuuaaagaugauuuagauucauucucuaauaaagaguuauccaauuuaauuauagaugauauugauguugcgaaauuagaugauucaauccccgcagauacuguaauuaaagccuuggauaugucgcaaaugacugugcauagugauuggaauaagaaagacuuugcuaaaaacaaaguaccuacauugguccauagaauguuggauaugcgcguucauagcgauaaaucccaggauaauaagaaaauuucuaggaaggcuaaccguguggagagaauuuuagaaaauaugacaguuaaaggugaauucgauuuuauuaaacauaaaauuacaaacucucucggcaagguuguuguuuccgcaagcgguguaguuaagacggucguagguaugaacauuaaaggacauuauguaauuuguccagcacauguuuauccaguugauacagcguuccauuaugcaaauuuuaccccguuuaauacggguaaaaauuacacaaugguaccaauggucuguaguagaguagaugauuuggcuuuguuuuacauaacugauaaauuacauccauuagcgaaagauauuacacaucauuuugaugaucaggaacguuuauauacucgaccauucgaguugccuuauaugcauuuacgaacugguaauguaguaucuuggaaagguggagaugcuauuggauugcgaucauugguuacaaaaguucaucaagacauguuauuaacagaaggaaaaccuaugguagcaggucaauuuuggaguacgguuacucgccuuacuaaaggcgauuguggauucccaauaauagauacuaacucucauaaaauauuuucuauuuauuguggagauaauaauggcguuaaugcuauuuauggaaauaugguaacaucaacauugguuaagaaaaugauugacuuugcugaagguguaucugaaguacacgcuuuuacucguuauaguccacccauggaagaguucuuugaugcagaagauacugguguuaugaaguuuuccuauccaucuggagaacaacauauuguuaugccuguugaaacuugugauuauaucucaaaagcauaugaaacugaaaacuauuacccuugcauggauggagaauuuacgaggaauaucgcguacaguagugauugucaaaagaacgcauuuaugaaaccuaaauaucaacgaacaccuuuugcugaagaaauuuuaacucaauauccacaaauuaaagaugauuguagaaaugccgcuacugacccaagguuaguagaagauaaaucaaaauuaguaaauucaguuaaaggagacccaucaauuguuaugacacaacuggcaaagauuaaugaccccauagaugcagcugguggacuuaugggugagaaguuuauggaagaagcagcugaaauguuaacagaagaguauauugacuuguauagcgguuuugaccauagauuuuugacuacagaagaaacgauaaaugguauugcaaguccucaaucggauuuacaaggucauuuagaagcuguaaauagagaugcgucugcaggucauugguguaugcuaaauggauaucauaaaauuucugauuuauuagaagaaggugguaguuacccuucaggucaacuacgguaucauuuuaagaaagagaauaaaguaauugcuaaauuagaaaaacauauaucacaacaaaagaagaugugggcaaaagguauucguuacgaagguuuaacuaaagauaauuuaaaagccgaauuaagaccauuugaaaaagugagaauuggaaaaacgcguguauuugaaaguuuugacuucuuuacaaccuuuaaccauagacaacuauuuggaacgauacaagcugcaaugaagaagcagagagcucggggaacaugucaaauuggauuagcucaauaugauuuuacgcacuuguauaauagauugcugcagaccucuccauuugcuguucacggggacuucgaggccugggauaagcauauggauugguuuucaguacaguuggcuuguaagauguggacgaagguuuuauggaaaucaccacaauuccaacauuggaaugauaaaauaaaugcacacaaauuugaaugugaagaaaauuuauaccugauguuggaaacugcaauguuuucuaauguguaugcuguuacuuuggcagauggacauauaauaguuaaagaaagaucucaauguucaggaguaguagaaacgacaacuuuaaauucuacuguuaaugacauuaaguguguggcuguuauauuguaucugauaaagaaacauaacguggugcuuagaacucuaggugcuucggaagcagaugcaaggaaguacuaugaagauuacuuggaaacauuggagcuuucaaagaagaguagaaaagcuuuauuaacaucgaaacauauuuauucaccgauuauaccagauagaagaacgauuaaacaaaacuuugauaugauauauuauggcgaugauauugguauuguaguaaauuuagaauuucuguuuauuguaaauuuuguuucaauuaaaaaugcuuauaaauacuuguucgguauuaauuuggacucaccucauaaagaugggucauuaguaccuuuugugccuuuaaaugaauuggaauuuguuaguagaacuuuugauuaucauucugauacuagaaccguuuauccuaaauuaaaagaacaaucaauuuggagacuuuuguucugguguacuaauuuacguccagagcagauagaauccaauaugauugcaauggcuaacgaauuguuccuuuaugaagaaaguaaauacagauuguuuaguucaauugucaacgcaauaauaucaccauaucugaaacgccaauauaauuggaauuugauauuaccaaguuauagacuuggaaggguagcauuugaggagaugucgcaugaacugaaggaggugccucacucgagcaccuguuuuacugaauuggagauucauucaauuuuacagaaagcgcaaaauauaggaagcaacugacuaaggacacuuccucuuuuguaauccguaaagaucgaacaaaugauuauaauggcgucaccaaaaacuuuacaaaaaauguuuaacaccuuuauagugcggaauggguuaccacuagaauuaucugguaccccugcuuucuuaaccgcagaugagcagaauguagacuccauuuggaguguuucauucaaaccgcuuauugaagcgccucuugcuguguacaagguuguccuccagagagcuugcucaaaggaggaaaacauuguauacaaaaaggcauauaguguuucuguaagcauugauucaaacacuuggauugacagaauacaacugcaauguguuuugcccggcauagaaccaccugugucgaguaaucaaauaauacaaaaacaacaaauagauuuaucacaguggaguggcauuuuccaagagggacuuucaaauuuggaaguccauucuaaagcugugacaacaaagacauccgcugcaaccucauuuaauauaauaucaaauccaacggagcaugguacaccaauacccauuacugguagugacgcaccaggcguaccuuuggauuuauuaucugguauggucuaugcacagaaaaacccaacuauuuaugcucaugaaccacaaauaguauauagagguaccaucucaucuaaagccgcuagaggugauguuuuauuagacuuggaucuggguaaauuaauuaaccagaauauggugucugcaacuaaggauuguucaacauucaaggguucuauaauuggaacuguugaaauguuggcuaauccagugguugcaggugcaauuaugguugguuauguaccgccuggaacaacuaauauaacagaagaaguuaugauggguacugaauguggcguuguuagugcuacggacagucaauuuuucucuuuuguauuaaaaccauauauuaaaaguacugguucaccacaagguagauauggauggcuaaauuuugauacuacaacaggagaauauacgcucccaaagacgacaaaggaugcuuauccacauuuagugaucauagcaagaaaugcguuuguugccuuuaauacggcaacagaagcaguaguuaaugucacuguaaugucuucuuuuggaccggauuucuucgcauuuucagaaggaccagaaauaacaggacugaaauuaacagauagugcaugggucgcaccaaaaagugaugucuuuggagcaucuagaucaguauuaaccaguuaugguuuggaaccaaaugcaguaauugcuauugauggcagggcaacgccaaaagaaagaauugucuauccaucagugaucuugcacuuugcaagcugugacacguucaaugaaggagcgacgauuaagaauguugguauguggacauauggagcaaauacuguaguugguucuccaggagugagaagcuauggaacauguuuucuugaaauuacaacuagagaggauacugggaaugaaacaucuuugacuaugcaacuacacuugggcacagcaacagauccuuauaauucauuuggcgaagcuaauacucaauauaugcaagcugaaagauauuauggagauuuuaaagcaagagcuguugguguugucgagguguauuugacggccacugauauagaaccuucucuuaaaucauaugaauuugauguugagcuugauucaauuagaaaagcaacuguaacaagacauguugguaauuuaucuucugaugggacaauuaauaauuauuauaugguauuugauaacuaugaaucaguagugaaaguuuuggacauuccuuuaccaggcuuuaaaaguaugacauacguugugaagaguuaugcagucaauguaacauucgauuuuucaucuacaucacuuauuagaggaaucacuaaucaagauaucaccauuuuaccuggaaauaguugcgauguuugugugauagaaccuaaggaugguguucugccuaauauuccgcagacuggcauccuuacggauaauuauaauacuggaccuucauuguuagauugcccaagucuuuaucauuauuuguauccugcauuuaaugagaauuuaguuagauuugacuugguugaacuuguaacgaauacagucauggcaacuaugguuuaucauccuaaaauuaaaaccguauucacaacucauagaacggcuggaaguaaugauucacaaugcuauauggucucacgauacuyugcaggacaaauggyucugucuaauguucaguugcucguggauaaugguaacuaucccagcacgcaagacguuguugguuucguuaccaggcucuugggcgauucuacagcauugguuagaggcauugaaaguaugaauguucacgguaaugcugaagcucuuuugggcgccagcacugccguuaaugcucuacaaccucuugcaaauuauuaucagaacaaagcgaaucaagauugguauuggaaacaaaagcaagcagaauaugaacaagaacaagcccuuuuggcagcaaaaaauaaaaacgcuuuagauauugcaaauguucaagcucaaaaucaacuugacuuacaaagagccaaacuugacucccaauacggguuggcacccagcaauggugugucgacuagugucaauucuucaucgacaguuccuuauguauaucauucgacacuugguaguaauguuccauccaauccuagugacucugaugagaaugauguuucucgucgagccauuaguaugcgugaugguaagauaaacauaagugaaggcaauagaguacaaccugguccaucuacaacauccaguguuucaaccucugaugagaauacgggagguagaguaauuuacccgacuucgacgaacccugauuugacaacaaugauacagcagacugcaaauaagguuaaaggagggaauguugaugcaaauggagaccuugugccuucaggcaaucuggcuucauuauugaaagaggaugaauacagaaugcccaauuuggaugcacaggaccucauugaugcuaaagcugacccugcacaaaauuuggcuuuucagagugauuacucuuauuacaauucuccugauacuucaacagauggucuuggcguaacaccacaaggaccucauuaucaaguagaggauccacaagaaaacccgccgccaguuccaaaaacauggauggagaaguggaauuugaguaaacacgcuaaaucaagugguuuacagucugcuguacaaaaauuacaacccaaauaggguaaccuauuuggggaccucacuuuuaguugcuagcuaacaaugggggccuuaaaauuaucuuauuaccuuaauugguuguaagugcugaauuuuaagggggccgcgcauauugcguaguaagaccugggccuugcauguagcauuauaagucccacucguuauagcuuaaaucguaccuuagcccguacgagacaagaagugaauuauauuuaaccaccauauauuaaucauuuaguauauaagguagauauuagaaauuaaauaauauagaaaugaugaauaaccuaucagcuugguaauugccuaaaugcgucuguuuucugugucaauuuuagcguaagcuggccuugacacuuuuaaccggugcaugaauagauuauuuuuaaaauuuguauuuuuacguaaguuugccuacaaaauuuuacauuucgaaacaaucaugaauagaauaacaguaaaagugcguuagucacguggcgcaccuauauuaacuuuuuuaacguuuauuucauuaguauagguuauucauuauaauauauuguacauauacuauaauucccaaauuuauguuuguuauuguacacccccgauuagucgguuuauauuuauauauuuggcucugcgaaugcaguuaaacgcuaccacuuuucggggacuaucuaaucuuuauauaaaauuaguuagucggauuuuagggccuucagcccucuaaucuuuuuauugaaaaaaaaaaaaaaaaaaaa

SEQ ID NO: 89 - protein, 549 aa, Dicistrovirus DVVv3
AATDPRLVEDKSKLVNSVKGDPSIVMTQLAKINDPIDAAGGLMGEKFMEEAAEMLTEEYIDLYSGFDHRFLTTEETINGIASPQSDLQGHLEAVNRDASAGHWCMLNGYHKISDLLEEGGSYPSGQLRYHFKKENKVIAKLEKHISQQKKMWAKGIRYEGLTKDNLKAELRPFEKVRIGKTRVFESFDFFTTFNHRQLFGTIQAAMKKQRARGTCQIGLAQYDFTHLYNRLLQTSPFAVHGDFEAWDKHMDWFSVQLACKMWTKVLWKSPQFQHWNDKINAHKFECEENLYLMLETAMFSNVYAVTLADGHIIVKERSQCSGVVETTTLNSTVNDIKCVAVILYLIKKHNVVLRTLGASEADARKYYEDYLETLELSKKSRKALLTSKHIYSPIIPDRRTIKQNFDMIYYGDDIGIVVNLEFLFIVNFVSIKNAYKYLFGINLDSPHKDGSLVPFVPLNELEFVSRTFDYHSDTRTVYPKLKEQSIWRLLFWCTNLRPEQIESNMIAMANELFLYEESKYRLFSSIVNAIISPYLKRQYNWNLILPSYR

SEQ ID NO: 90 - protein, 1073 aa, Dicistrovirus DVVv3
  misc_feature (728)..(728): The ′Xaa′ at location 728 stands for Ser, or Phe.
  misc_feature (733)..(733): The ′Xaa′ at location 733 stands for Ala, or Val.
MIIMASPKTLQKMFNTFIVRNGLPLELSGTPAFLTADEQNVDSIWSVSFKPLIEAPLAVYKVVLQRACSKEENIVYKKAYSVSVSIDSNTWIDRIQLQCVLPGIEPPVSSNQIIQKQQIDLSQWSGIFQEGLSNLEVHSKAVTTKTSAATSFNIISNPTEHGTPIPITGSDAPGVPLDLLSGMVYAQKNPTIYAHEPQIVYRGTISSKAARGDVLLDLDLGKLINQNMVSATKDCSTFKGSIIGTVEMLANPVVAGAIMVGYVPPGTTNITEEVMMGTECGVVSATDSQFFSFVLKPYIKSTGSPQGRYGWLNFDTTTGEYTLPKTTKDAYPHLVIIARNAFVAFNTATEAVVNVTVMSSFGPDFFAFSEGPEITGLKLTDSAWVAPKSDVFGASRSVLTSYGLEPNAVIAIDGRATPKERIVYPSVILHFASCDTFNEGATIKNVGMWTYGANTVVGSPGVRSYGTCFLEITTREDTGNETSLTMQLHLGTATDPYNSFGEANTQYMQAERYYGDFKARAVGVVEVYLTATDIEPSLKSYEFDVELDSIRKATVTRHVGNLSSDGTINNYYMVFDNYESVVKVLDIPLPGFKSMTYVVKSYAVNVTFDFSSTSLIRGITNQDITILPGNSCDVCVIEPKDGVLPNIPQTGILTDNYNTGPSLLDCPSLYHYLYPAFNENLVRFDLVELVTNTVMATMVYHPKIKTVFTTHRTAGSNDSQCYMVSRYXAGQMXLSNVQLLVDNGNYPSTQDVVGFVTRLLGDSTALVRGIESMNVHGNAEALLGASTAVNALQPLANYYQNKANQDWYWKQKQAEYEQEQALLAAKNKNALDIANVQAQNQLDLQRAKLDSQYGLAPSNGVSTSVNSSSTVPYVYHSTLGSNVPSNPSDSDENDVSRRAISMRDGKINISEGNRVQPGPSTTSSVSTSDENTGGRVIYPTSTNPDLTTMIQQTANKVKGGNVDANGDLVPSGNLASLLKEDEYRMPNLDAQDLIDAKADPAQNLAFQSDYSYYNSPDTSTDGLGVTPQGPHYQVEDPQENPPPVPKTWMEKWNLSKHAKSSGLQSAVQKLQPK

SEQ ID NO: 91 - RNA, 675 nt, Dicistrovirus DVVv3
ccauuacggccgggggaaaauaagggcuauggcgucaugccauaguuuauauacaccacggauuacgugaaccuuguuaguaauaacgggugcccuuuguuagggauguguuuuaugaauuauuaauuauguuauaacucuuauuuucaacuacgggauuaaaaccccacuuuaaaacuauauagggaaauuuaguuuaaaccuguauaggaaauuaaggauugauaauuuuauugaacgagccuacucguugaauauuuauaguuaguugugauaacuagccuuguauuuagacauggauaaucuccagcccauguucgacauaccuuaggaaguuuauaugcggcacguauucaauuaaaugaagaugcuauaagugcuugauuagacauuaaaacuagaacaaguucucuaguugucgcugguagagcauaacuuauaguagguggauuuuauuugaugacccaaguauuuaugaaauuugucggccuccaaguugaucagcuaauuauuuauauuuugcgaguaauaguaguucagacugugcuauccccuugacaugcauuuuuugguuacuccagauugugauauuagggcucuaauauugcaagacugcccaccaaguaaggaacuuauguaaacuugcuugggaaacacauuacucaaaaaauuauu

SEQ ID NO: 92 - RNA, 746 nt, Dicistrovirus DVVv3
gguaaccuauuuggggaccucacuuuuaguugcuagcuaacaaugggggccuuaaaauuaucuuauuaccuuaauugguuguaagugcugaauuuuaagggggccgcgcauauugcguaguaagaccugggccuugcauguagcauuauaagucccacucguuauagcuuaaaucguaccuuagcccguacgagacaagaagugaauuauauuuaaccaccauauauuaaucauuuaguauauaagguagauauuagaaauuaaauaauauagaaaugaugaauaaccuaucagcuugguaauugccuaaaugcgucuguuuucugugucaauuuuagcguaagcuggccuugacacuuuuaaccggugcaugaauagauuauuuuuaaaauuuguauuuuuacguaaguuugccuacaaaauuuuacauuucgaaacaaucaugaauagaauaacaguaaaagugcguuagucacguggcgcaccuauauuaacuuuuuuaacguuuauuucauuaguauagguuauucauuauaauauauuguacauauacuauaauucccaaauuuauguuuguuauuguacacccccgauuagucgguuuauauuuauauauuuggcucugcgaaugcaguuaaacgcuaccacuuuucggggacuaucuaaucuuuauauaaaauuaguuagucggauuuuagggccuucagcccucuaaucuuuuuauugaaaaaaaaaaaaaaaaaaaa

SEQ ID NO: 93 - RNA, 42 nt, Dicistrovirus DVVv3
  misc_RNA (1)..(42): IGR
cuaaggacacuuccucuuuuguaauccguaaagaucgaacaa

SEQ ID NO: 94 - RNA, 10396 nt, synthetic construct
  5′UTR (1)..(675)
  CDS (676)..(9192): non-structural polyprotein, ORF1
  promoter (9193)..(9234): intergenic region (IGR)
  misc_RNA (9235)..(9649): Central region
  3′UTR (9651)..(10396)
ccauuacggccgggggaaaauaagggcuauggcgucaugccauaguuuauauacaccacggauuacgugaaccuuguuaguaauaacgggugcccuuuguuagggauguguuuuaugaauuauuaauuauguuauaacucuuauuuucaacuacgggauuaaaaccccacuuuaaaacuauauagggaaauuuaguuuaaaccuguauaggaaauuaaggauugauaauuuuauugaacgagccuacucguugaauauuuauaguuaguugugauaacuagccuuguauuuagacauggauaaucuccagcccauguucgacauaccuuaggaaguuuauaugcggcacguauucaauuaaaugaagaugcuauaagugcuugauuagacauuaaaacuagaacaaguucucuaguugucgcugguagagcauaacuuauaguagguggauuuuauuugaugacccaaguauuuaugaaauuugucggccuccaaguugaucagcuaauuauuuauauuuugcgaguaauaguaguucagacugugcuauccccuugacaugcauuuuuugguuacuccagauugugauauuagggcucuaauauugcaagacugcccaccaaguaaggaacuuauguaaacuugcuugggaaacacauuacucaaaaaauuauuauggaaaguucaaauaagaauauguguacuggauucccccaaacccuuaaaaucucaacggaaauuaguaucauaaaccaaucaucucaaacccccagagauuggucuuauguacaagaaaauaugcaacaguuuaaaaauuauuuacaauuagcauggauugaaguuccgaauaacauuacaauaggaaauuuugacguuuuugaagauaauaugccaauugcucacgcuauuucaucggauuugcaucaaacagcagguauugcugguguuuuaagacaaaauuauuuuaaaaaucuaccuaaaguuaacguaaauaaaaguaauauuggaaccuuauauuuguauccaaauaaaaauuuaacucgacaaauuauuggauugauuacuaaagauauuuauuauuuaaaaccaacuuaugcuucacuuuuuacaacuuuaauugaauugcguaaauaucuuauuaauaauaaaauaaaaaauauuugccuaccuaugguagguuguaauaaagauaaauuaaauccucuugcaguuuuacccaugcuaauguauugccucggcaauauugaaacucaaguaaaaauaacuaauaaaggaguuuauuuuaauuaucaaccaauuccuguugcuccaaaaauuaauaaaauuaaauuuaguaaacaaauggaaaaaguuaauuaugaucaaacuguagauacuuuugaaacaaauuauguuaaaccuaagaaauucaaagcuaaaguaacauuugaacccucucuuuuaaaucuugauucuaauaguauuauaauuaaugaaaguaagacaguaaaacaaauuuauaaagcuaaauuagaaaaaguaauauaucaaagaucuacaguuuaugaaccacaaaagaagacauaugugguagaagcuuuuucuuguaaacaaccuaaaccaauugucaaaauuuuuaaaaugaaacaagcaauuuuaacucaaacugacuuaaaagaaaauaauaaugaacuaauuaaagaaagaaauaguauugauuauuauaaauuuguuacaccuaacaccguuaaagcuauuaucggauacgacaaaguagaaucaguuuugacauuugauuuggaucguaaacguagauguggagaaaaagcuuuuguacauggaaaaucuuuaaaaucaauagcuaaacgacaacuguguuauaguuauguaccaucucuaaacucagugcaaguauauauuaaaauguuacaauuaacggcgcagaaacaaguauuuggucauguaguaacgaaaauucaagaaaguauugccaauauggauguucauggaaugcaaauuaacaauauaccugaagaauuaaugaaaucauuauccagaacaauuuuagaacaauuacaauuagacacuaaaguaauaaaaccaguuuuguuagcaaauauauuaaauuguuucguaaauguuguuugugccuuaauaauaauauucacugccaaaaguaaaguugcaucuggagcagcuauuguucaaauaauuucaaauauuuuaaccuuaauguuggauauaggcauuuauuauucacuuaaacauaugaccgaagaaagaauuaaagcaauaacuacgacuauaacacaggaugcuguccaaaaaguaaugaaacaacauggcguaacuaugauggcaucugauaaaguaauaacagccaugcaaaaugucgauuuaaaugaugcuaaaucaauaaagcauuuuauaaaagaauucgaaaaagaagaagauaccgacauuucagaaauuucacccgauuguguaccaaacuauucugauauguuaccuauuggauuacaaaugaaaaagaagaacaggcuugauauggauuuuguuuugauggacaaaauagcaguuguugauccaacggguaaguuaaugaagauaguuagugacuguccauacgacacaccuaguaaaaauaaucaguguuugcuccacucccuucugggugcacuagaucauagagauagaaaaguuuuaguggaugaaaguaauauugaaaaauucuuccaauggauguccgauaauauuagugauccagaugugauugaagcaauuaauguuuauaaaauggaguaugcuucugauuucccaccagaagcaacuaauuuagauguuuuacaaggaauuaaaacucauaagaaaauguuaccaguugaccuuuguaaauuacucucaaaauauuuggaccguacuauaauuguaaaacauguaaauaaaguuuugacuuauggaccggaaagaugggauaacaauauaccaaaaauacuccaauuagaaaaucaacauuuuagcuauauugaccgaguacuaguggauaauauuuugccauuaaaagcagcaguugaguguaugguucaauuuacuuuaacacgagccgcacaauuuaauacacccgauacacuuaaacaaaaagaguauuauuuagcgauggcagcagcauacguuuuuacauauaauucuagaauugcuguguguuguaugaaugugaaaucuaauaagcuugaugcgaaaucaauuauugguauuugcacagauauucugucuacaauugcuuucguaguugguaugggaguaucuaguggugauuuugcaucacgcaugguaaaggcugagaaauuuucaacaucuauggauacauuugcaucuaggacaacuaauaaguuagauucgauuuuaucgaaagcuuucgguauuuauaugucggaagauggcgaaacgguugcaaguuuauccaaagauauugauauacuagagaaauauaaagcacauacuguagccgauuauaugaauaauaugucaaaauaucaagaaguuaaaacauauguagaaggaguuacaaaacguaugacaucacuacaccuauuaaauagagauaccacuaaugcuauucaaaauacgcaaaauauuauaagaaagaguugugaugccauaauggcaguuuugaaugacguugauaaguugaauagaauggaaucauugagacaggaauuugugggccuuaagggagcugguaaaaccucaacauugaacaauuaucuaauaccaaauauugcuaagcgcuuggcguugaauccaagcaccuaugcagcagaaauugacgcgaauggacauuggaaacccuauagauaugaagauuuugcuuuaauagaugauuuguuuaaucaggguaguaacgaccccuuuauaccacauauuguaaggauggcuuccucucuuaacuuugauuuaccuggagcugacuugagcaauaaagagaaaacuuuaagggcuagauucuuguuugcgacagcaaacguugcauauguaagaaugaaggaagaaaugcccgcuagugcucaggaaggauuuuaugguagauuuaaguguauucaauuuauuaaugccaaacaagguauuaaucaggcaagagauucaguggaauacaucgacuaugaagaucaugauacauaugaauugagaauguaugaagaacaaauuaaugaacaauucucacagauugaaagaaaugaccauauaacuggugaacguauacaacauuuugauccuucuacuaagacuuaugaugaccauauuuacaaaauaggacauuuacauuauaaaauuguaaccuuuaaacaguuagaaacaauuuguauagaucaguuuaauaaaaaguaucaacauuacaaaauggcuagagaucguaaauguguaguggacuuugaauauuuuagaaauauugauaugaaaucagacgcaauaguagaaaauaugguacguaaggguuaucucccagcagaaauugaagcagcaagacaaacuguaggacuuacagcuggaccagaugaagugcaugccaaaucugaaucuauaauuaaucauuacguuguaucaguuuuuggcgauacuggaacuggaaaaucuguuuuugcuacacagcuggcguuagaucugucagacuuucugaauuugaaaguauauucuguggaagauauaaaugauacggcccauuuagacuacuuaaauccaaguauaauuguuuuccaugauagcauuuaugaugaauugaaauauguugcauuauaugauaaauuaccaaaaccauguuuaauaauaacuacaaguaaucauacauuaaaauggcauagagagaaauuauguaauauuguugauaauaguacuucagcagcagagcgcucuucuagugcuuuguuggaacaugguuucuuuaacaaaaugaaaaauaaaauuggcggacuuguuucuuauuuuaauuugucaaguauuagagaagguuauuugguucaaacauucccaggauaccaaccaacaccugguuauuguagaagacuagguuugaacacugcauuccacccgauaucgacagaagauuugacaucuaauuuaguauauucacagcguccuaauaaagaaacuacaaccauuuuugcaacuucaggauaccauuuccaucaggauaauaaagaagugcaauuaaacgaagucguuaaagaaguuuuaaugaaauauagaggagcuaagaaauucauucagccugauauuaucaaauuaggaguaucagaauuugaggaacaauuuaagaaauuaacaacuauugauguagaaauucagucuaaaaauauugagucauugauuaaauuaacgaacgauagagcuauacuaguaaacucuguaacugcagcuagauuuguuggugguucuaauauuguuaaaguuuccaguagagucagaucaaauaauuauucuuuuaauccucuagauuuugcuauuccuauuaaugcuacuguaucugaugcacuagauauagcaaugaaaucuuauaggcuauuauugagugcaaauguagauuauacaguauuuaucaaaugugaagaauuuucuuguauagguauuaacggcaauauuuacuauacuucaguaccuuuuaauguaacuucuuauucacauauugcaacuaaaguaauaacauuuuauaaaguagauacuaauucagauggaauaacaaauaauucagaauuaaucaaauuuacaccggaugaauaugcuaaauuaauuauuacaggagaaggagaacaaaaacuuuuugauaaguugccuuaugcagcuuguaaauauauuuauggcgcuagagcagagauugaaccacauuuugcgacaacauuuacuaaauacgaaguuagaacagaaguuuaugauaaucauacuagagagaaaauggcuuucuuuguaauuuauucagaauuuaagaaaacaauuugguauaaaauagcuauagguauuuuggcuauaacugcaucaguuuuaauaauugaauuaauaacacaaauuuggaauauguuuauugguuuaaaagaaguuuuuacaaguaccguucaaaaagauaauguuaaaguaguuuaucaaguuuuaucaguuaauccuaaagagcaaucagcagaaguuaaaguuuaugguuuuaaagaugauuuagauucauucucuaauaaagaguuauccaauuuaauuauagaugauauugauguugcgaaauuagaugauucaauccccgcagauacuguaauuaaagccuuggauaugucgcaaaugacugugcauagugauuggaauaagaaagacuuugcuaaaaacaaaguaccuacauugguccauagaauguuggauaugcgcguucauagcgauaaaucccaggauaauaagaaaauuucuaggaaggcuaaccguguggagagaauuuuagaaaauaugacaguuaaaggugaauucgauuuuauuaaacauaaaauuacaaacucucucggcaagguuguuguuuccgcaagcgguguaguuaagacggucguagguaugaacauuaaaggacauuauguaauuuguccagcacauguuuauccaguugauacagcguuccauuaugcaaauuuuaccccguuuaauacggguaaaaauuacacaauggucccaauggucuguaguagaguagaugauuuggcuuuguuuuacauaacugauaaauuacauccauuagcgaaagauauuacacaucauuuugaugaucaggaacguuuauauacucgaccauucgaguugccuuauaugcauuuacgaacugguaauguaguaucuuggaaagguggagaugcuauuggauugcgaucauugguuacaaaaguucaucaagacauguuauuaacagaaggaaaaccuaugguagcaggucaauuuuggaguacgguuacucgccuuacuaaaggcgauuguggauucccaauaauagauacuaacucucauaaaauauuuucuauuuauuguggagauaauaauggcguuaaugcuauuuauggaaauaugguaacaucaacauugguuaagaaaaugauugacuuugcugaagguguaucugaaguacacgcuuuuacucguuauaguccacccauggaagaguucuuugaugcagaagauacugguguuaugaaguuuuccuauccaucuggagaacaacauauuguuaugccuguugaaacuugugauuauaucucaaaagcauaugaaacugaaaacuauuacccuugcauggauggagaauuuacgaggaauaucgcguacaguagugauugucaaaagaacgcauuuaugaaaccuaaauaucaacgaacaccuuuugcugaagaaauuuuaacucaauauccacaaauuaaagaugauuguagaaaugccgcuacugacccaagguuaguagaagauaaaucaaaauuaguaaauucaguuaaaggagacccaucaauuguuaugacacaacuggcaaagauuaaugaccccauagaugcagcugguggacuuaugggugagaaguuuauggaagaagcagcugaaauguuaacagaagaguauauugacuuguauagcgguuuugaccauagauuuuugacuacagaagaaacgauaaaugguauugcaaguccucaaucggauuuacaaggucauuuagaagcuguaaauagagaugcgucugcaggucauugguguaugcuaaauggauaucauaaaauuucugauuuauuagaagaaggugguaguuacccuucaggucaacuacgguaucauuuuaagaaagagaauaaaguaauugcuaaauuagaaaaacauauaucacaacaaaagaagaugugggcaaaagguauucguuacgaagguuuaacuaaagauaauuuaaaagccgaauuaagaccauuugaaaaagugagaauuggaaaaacgcguguauuugaaaguuuugacuucuuuacaaccuuuaaccauagacaacuauuuggaacgauacaagcugcaaugaagaagcagagagcucggggaacaugucaaauuggauuagcucaauaugauuuuacgcacuuguauaauagauugcugcagaccucuccauuugcuguucacggggacuucgaggccugggauaagcauauggauugguuuucaguacaguuggcuuguaagauguggacgaagguuuuauggaaaucaccacaauuccaacauuggaaugauaaaauaaaugcacacaaauuugaaugugaagaaaauuuauaccugauguuggaaacugcaauguuuucuaauguguaugcuguuacuuuggcagauggacauauaauaguuaaagaaagaucucaauguucaggaguaguagaaacgacaacuuuaaauucuacuguuaaugacauuaaguguguggcuguuauauuguaucugauaaagaaacauaacguggugcuuagaacucuaggugcuucggaagcagaugcaaggaaguacuaugaagauuacuuggaaacauuggagcuuucaaagaagaguagaaaagcuuuauuaacaucgaaacauauuuauucaccgauuauaccagauagaagaacgauuaaacaaaacuuugauaugauauauuauggcgaugauauugguauuguaguaaauuuagaauuucuguuuauuguaaauuuuguuucaauuaaaaaugcuuauaaauacuuguucgguauuaauuuggacucaccucauaaagaugggucauuaguaccuuuugugccuuuaaaugaauuggaauuuguuaguagaacuuuugauuaucauucugauacuagaaccguuuauccuaaauuaaaagaacaaucaauuuggagacuuuuguucugguguacuaauuuacguccagagcagauagaauccaauaugauugcaauggcuaacgaauuguuccuuuaugaagaaaguaaauacagauuguuuaguucaauugucaacgcaauaauaucaccauaucugaaacgccaauauaauuggaauuugauauuaccaaguuauagacuuggaaggguagcauuugaggagaugucgcaugaacugaaggaggugccucacuccagcaccuguuuuacugaauuggagauucauucaauuuuacagaaagcgcaaaauauaggaagcaacugacuaaggacacuuccucuuuuguaauccguaaagaucgaacaaccuaggcauaaucaagguaccccggagcgacggugaaggacggguccaguaguucgcuacuguugaguagagugugagcuccgucgcaccggaacaaggcacguaggagacggucggguccagauauucguaucugucgaguagagugugggcuccuacgugcuuauuggacaugcggagacggucggguccagauauucguaucugucgaguagagugugggcuccgcauguccuuauugggugcauggagacggucggguccagauauucguaucugucgaguagagugugggcuccaugcacccuuauugacgccaacugaaugaaauggugaaggacggguccagguguggcugcuucggcagugcagcuuguugaguagagugugagcuccguaacuagucgcgucuuauugguaaccuauuuggggaccucacuuuuaguugcuagcuaacaaugggggccuuaaaauuaucuuauuaccuuaauugguuguaagugcugaauuuuaagggggccgcgcauauugcguaguaagaccugggccuugcauguagcauuauaagucccacucguuauagcuuaaaucguaccuuagcccguacgagacaagaagugaauuauauuuaaccaccauauauuaaucauuuaguauauaagguagauauuagaaauuaaauaauauagaaaugaugaauaaccuaucagcuugguaauugccuaaaugcgucuguuuucugugucaauuuuagcguaagcuggccuugacacuuuuaaccggugcaugaauagauuauuuuuaaaauuuguauuuuuacguaaguuugccuacaaaauuuuacauuucgaaacaaucaugaauagaauaacaguaaaagugcguuagucacguggcgcaccuauauuaacuuuuuuaacguuuauuucauuaguauagguuauucauuauaauauauuguacauauacuauaauucccaaauuuauguuuguuauuguacacccccgauuagucgguuuauauuuauauauuuggcucugcgaaugcaguuaaacgcuaccacuuuucggggacuaucuaaucuuuauauaaaauuaguuagucggauuuuagggccuucagcccucuaaucuuuuuauugaaaaaaaaaaaaaaaaaaaa

SEQ ID NO: 95 - protein, 2838 aa, synthetic construct
MESSNKNMCTGFPQTLKISTEISIINQSSQTPRDWSYVQENMQQFKNYLQLAWIEVPNNITIGNFDVFEDNMPIAHAISSDLHQTAGIAGVLRQNYFKNLPKVNVNKSNIGTLYLYPNKNLTRQIIGLITKDIYYLKPTYASLFTTLIELRKYLINNKIKNICLPMVGCNKDKLNPLAVLPMLMYCLGNIETQVKITNKGVYFNYQPIPVAPKINKIKFSKQMEKVNYDQTVDTFETNYVKPKKFKAKVTFEPSLLNLDSNSIIINESKTVKQIYKAKLEKVIYQRSTVYEPQKKTYVVEAFSCKQPKPIVKIFKMKQAILTQTDLKENNNELIKERNSIDYYKFVTPNTVKAIIGYDKVESVLTFDLDRKRRCGEKAFVHGKSLKSIAKRQLCYSYVPSLNSVQVYIKMLQLTAQKQVFGHVVTKIQESIANMDVHGMQINNIPEELMKSLSRTILEQLQLDTKVIKPVLLANILNCFVNVVCALIIIFTAKSKVASGAAIVQIISNILTLMLDIGIYYSLKHMTEERIKAITTTITQDAVQKVMKQHGVTMMASDKVITAMQNVDLNDAKSIKHFIKEFEKEEDTDISEISPDCVPNYSDMLPIGLQMKKKNRLDMDFVLMDKIAVVDPTGKLMKIVSDCPYDTPSKNNQCLLHSLLGALDHRDRKVLVDESNIEKFFQWMSDNISDPDVIEAINVYKMEYASDFPPEATNLDVLQGIKTHKKMLPVDLCKLLSKYLDRTIIVKHVNKVLTYGPERWDNNIPKILQLENQHFSYIDRVLVDNILPLKAAVECMVQFTLTRAAQFNTPDTLKQKEYYLAMAAAYVFTYNSRIAVCCMNVKSNKLDAKSIIGICTDILSTIAFVVGMGVSSGDFASRMVKAEKFSTSMDTFASRTTNKLDSILSKAFGIYMSEDGETVASLSKDIDILEKYKAHTVADYMNNMSKYQEVKTYVEGVTKRMTSLHLLNRDTTNAIQNTQNIIRKSCDAIMAVLNDVDKLNRMESLRQEFVGLKGAGKTSTLNNYLIPNIAKRLALNPSTYAAEIDANGHWKPYRYEDFALIDDLFNQGSNDPFIPHIVRMASSLNFDLPGADLSNKEKTLRARFLFATANVAYVRMKEEMPASAQEGFYGRFKCIQFINAKQGINQARDSVEYIDYEDHDTYELRMYEEQINEQFSQIERNDHITGERIQHFDPSTKTYDDHIYKIGHLHYKIVTFKQLETICIDQFNKKYQHYKMARDRKCVVDFEYFRNIDMKSDAIVENMVRKGYLPAEIEAARQTVGLTAGPDEVHAKSESIINHYVVSVFGDTGTGKSVFATQLALDLSDFLNLKVYSVEDINDTAHLDYLNPSIIVFHDSIYDELKYVALYDKLPKPCLIITTSNHTLKWHREKLCNIVDNSTSAAERSSSALLEHGFFNKMKNKIGGLVSYFNLSSIREGYLVQTFPGYQPTPGYCRRLGLNTAFHPISTEDLTSNLVYSQRPNKETTTIFATSGYHFHQDNKEVQLNEVVKEVLMKYRGAKKFIQPDIIKLGVSEFEEQFKKLTTIDVEIQSKNIESLIKLTNDRAILVNSVTAARFVGGSNIVKVSSRVRSNNYSFNPLDFAIPINATVSDALDIAMKSYRLLLSANVDYTVFIKCEEFSCIGINGNIYYTSVPFNVTSYSHIATKVITFYKVDTNSDGITNNSELIKFTPDEYAKLIITGEGEQKLFDKLPYAACKYIYGARAEIEPHFATTFTKYEVRTEVYDNHTREKMAFFVIYSEFKKTIWYKIAIGILAITASVLIIELITQIWNMFIGLKEVFTSTVQKDNVKVVYQVLSVNPKEQSAEVKVYGFKDDLDSFSNKELSNLIIDDIDVAKLDDSIPADTVIKALDMSQMTVHSDWNKKDFAKNKVPTLVHRMLDMRVHSDKSQDNKKISRKANRVERILENMTVKGEFDFIKHKITNSLGKVVVSASGVVKTVVGMNIKGHYVICPAHVYPVDTAFHYANFTPFNTGKNYTMVPMVCSRVDDLALFYITDKLHPLAKDITHHFDDQERLYTRPFELPYMHLRTGNVVSWKGGDAIGLRSLVTKVHQDMLLTEGKPMVAGQFWSTVTRLTKGDCGFPIIDTNSHKIFSIYCGDNNGVNAIYGNMVTSTLVKKMIDFAEGVSEVHAFTRYSPPMEEFFDAEDTGVMKFSYPSGEQHIVMPVETCDYISKAYETENYYPCMDGEFTRNIAYSSDCQKNAFMKPKYQRTPFAEEILTQYPQIKDDCRNAATDPRLVEDKSKLVNSVKGDPSIVMTQLAKINDPIDAAGGLMGEKFMEEAAEMLTEEYIDLYSGFDHRFLTTEETINGIASPQSDLQGHLEAVNRDASAGHWCMLNGYHKISDLLEEGGSYPSGQLRYHFKKENKVIAKLEKHISQQKKMWAKGIRYEGLTKDNLKAELRPFEKVRIGKTRVFESFDFFTTFNHRQLFGTIQAAMKKQRARGTCQIGLAQYDFTHLYNRLLQTSPFAVHGDFEAWDKHMDWFSVQLACKMWTKVLWKSPQFQHWNDKINAHKFECEENLYLMLETAMFSNVYAVTLADGHIIVKERSQCSGVVETTTLNSTVNDIKCVAVILYLIKKHNVVLRTLGASEADARKYYEDYLETLELSKKSRKALLTSKHIYSPIIPDRRTIKQNFDMIYYGDDIGIVVNLEFLFIVNFVSIKNAYKYLFGINLDSPHKDGSLVPFVPLNELEFVSRTFDYHSDTRTVYPKLKEQSIWRLLFWCTNLRPEQIESNMIAMANELFLYEESKYRLFSSIVNAIISPYLKRQYNWNLILPSYRLGRVAFEEMSHELKEVPHSSTCFTELEIHSILQKAQNIGSN

SEQ ID NO: 96 - RNA, 416 nt, synthetic construct
  misc_RNA (1)..(416): Contains an AvrII/KpnI MCS and 3 broccoli fluorescence aptamers.
ccuaggcauaaucaagguaccccggagcgacggugaaggacggguccaguaguucgcuacuguugaguagagugugagcuccgucgcuccgguuauuggcacguaggagacggucggguccagauauucguaucugucgaguagagugugggcuccuacgugcuuauuggacaugcggagacggucggguccagauauucguaucugucgaguagagugugggcuccgcauguccuuauugggugcauggagacggucggguccagauauucguaucugucgaguagagugugggcuccaugcacccuuauugacgccaacugaaugaaauggugaaggacggguccagguguggcugcuucggcagugcagcuuguugaguagagugugagcuccguaacuagucgcgucuuauu

SEQ ID NO: 97 - RNA, 2245 nt, synthetic construct
  misc_feature (1)..(2245): Central region
  misc_feature (25)..(129): MV-RNA Mango I
  misc_feature (135)..(563): (4) MV-RNA targeting WCR vATPase gene
  misc_feature (569)..(678): MV-RNA Mango II
  misc_feature (684)..(1109): (4) MV-RNA targeting WCR Sec23 gene
  misc_feature (1115)..(1218): MV-RNA Mango III
  misc_feature (1224)..(1674): (4) MV-RNA targeting WCR Snakeskin gene
  misc_feature (1680)..(1789): MV-RNA Mango IV
  misc_feature (1795)..(2215): (4) MV-RNA targeting WCR DVsnf7 gene
cuagggcaacgccggucuuauuauacgccaaacaagcuuugccgaucugagccugggagcucucggcaaagcagcuugagaucgaaggagaggagaggaagaggagaguaucucaagcauguuuggcguauuauaccagcacguucguagaagggaucugagccugggagcucucccuucuacgucuaacaugcccucguguagcucauuagcuccgagggcauguuagauguguugguauuauaaauuucucucaaagcuucgggaucugagccugggagcucucccgaagcuuaauugacugcucguguagcucauuagcuccgagcagucaauucagggaaauuuauuaucuuacguugggcaaguuucgaucugagccugggagcucucgaaauuugcgccgaagcuucucguguagcucauuagcuccgagaagcuucggcccaacgugagauuaucuuguccaaaccccagaacgaucugagccugggagcucucguucuggggcguaagcacucucguguagcucauuagcuccgagagugcuuacguugggcaagauuaucggccacacggauccugcgcucguguagcucauuagcuccgagcgcaggauaauccgagauaccgagggaguggugaggaugaggcgaguaucucggaugcguguggccgauuauuuugcaaaauuucuugugcagaucugagccugggagcucucugcauaagagguucccaacucguguagcucauuagcuccgaguugggaaccuaguuugcaaaauuauacuugaagggauacaucaucgaucugagccugggagcucucgaugauguaccccuuggucucguguagcucauuagcuccgagaccaaggggcucuucacguauuauuguguauucaaugguggaaagaucugagccugggagcucucuuuccaccaacgccaagucucguguagcucauuagcuccgagacuuggcguuaaaguacacaauuauccuaguuugcaaaauuucuugaucugagccugggagcucucaagaaauuuauaucaagacucguguagcucauuagcuccgagucuugauauuuuaaacuacgauuauacgccagccacuaguugcggaucugagccugggagcucuccgcaacuaauagugagcucgaaggagaggagaggaagaggagagagcucacuaauggcuggcguauuauugcugaaguucuuugcuucagaucugagccugggagcucucugaagcaaaggcugaucaguuucucguguagcucauuagcuccgagaaauugaucaccaacuucagcaauuauuggaaucuaauuauauuuaugaucugagccugggagcucucauaaauauaauaugaagagaacucguguagcucauuagcuccgaguuuucuucguuuagauuccaauuauaagccaaaauggauucaauugaucugagccugggagcucucgauugaauccgguuaauagcuucucguguagcucauuagcuccgagaaguuauugcauuuuggcuuauuauguuauauacuaaaugcaauagaucugagccugggagcucucuauugcauuuuggcuuugagcacucguguagcucauuagcuccgagugcuuaaaguaaaguauauaacauuauauuaucggccacgcucuagaugcgcucguguagcucauuagcuccgagcgcaucuauuagagagagaccgagggaguggugaggaugaggcgagucucucucuaaagcguggccgauuauuccaugauaucgugaacaugaucugagccugggagcucucauguucacgucauggaugcucguguagcucauuagcuccgagcauccaugauaucgugaaauuauaggggcuaucucuuccuuugaucugagccugggagcucucaaaggaagacacaaauaccucguguagcucauuagcuccgagguauuugugcuagcuccuauuauaccaugguaaauucuucuaugaucugagccugggagcucucauagaagaacaaagaaaacucguguagcucauuagcuccgaguuuucuuugcuaccaugguauuauuguaguugggucuuuuccaggaucugagccugggagcucuccuggaaaagacuaaaccaacucguguagcucauuagcuccgaguugguuuaguagcaacugcaauuaugaccggcguugccuuaggccgguac

SEQ ID NO: 98 - RNA, 1879 nt, synthetic construct
  5′UTR (1)..(675)
  misc_signal (676)..(717)
  misc_feature (719)..(737): AvrII/KpnI MCS site
  misc_feature (719)..(1128): Central region
  3′UTR (1134)..(1879)
ccauuacggccgggggaaaauaagggcuauggcgucaugccauaguuuauauacaccacggauuacgugaaccuuguuaguaauaacgggugcccuuuguuagggauguguuuuaugaauuauuaauuauguuauaacucuuauuuucaacuacgggauuaaaaccccacuuuaaaacuauauagggaaauuuaguuuaaaccuguauaggaaauuaaggauugauaauuuuauugaacgagccuacucguugaauauuuauaguuaguugugauaacuagccuuguauuuagacauggauaaucuccagcccauguucgacauaccuuaggaaguuuauaugcggcacguauucaauuaaaugaagaugcuauaagugcuugauuagacauuaaaacuagaacaaguucucuaguugucgcugguagagcauaacuuauaguagguggauuuuauuugaugacccaaguauuuaugaaauuugucggccuccaaguugaucagcuaauuauuuauauuuugcgaguaauaguaguucagacugugcuauccccuugacaugcauuuuuugguuacuccagauugugauauuagggcucuaauauugcaagacugcccaccaaguaaggaacuuauguaaacuugcuugggaaacacauuacucaaaaaauuauucuaaggacacuuccucuuuuguaauccguaaagaucgaacaaccuaggcauaaucaagguaccccggagcgacggugaaggacggguccaguaguucgcuacuguugaguagagugugagcuccgucgcuccgguuauuggcacguaggagacggucggguccagauauucguaucugucgaguagagugugggcuccuacgugcuuauuggacaugcggagacggucggguccagauauucguaucugucgaguagagugugggcuccgcauguccuuauugggugcauggagacggucggguccagauauucguaucugucgaguagagugugggcuccaugcacccuuauugacgccaacugaaugaaauggugaaggacggguccagguguggcugcuucggcagugcagcuuguugaguagagugugagcuccguaacuagucgcgucuuauugguaaccuauuuggggaccucacuuuuaguugcuagcuaacaaugggggccuuaaaauuaucuuauuaccuuaauugguuguaagugcugaauuuuaagggggccgcgcauauugcguaguaagaccugggccuugcauguagcauuauaagucccacucguuauagcuuaaaucguaccuuagcccguacgagacaagaagugaauuauauuuaaccaccauauauuaaucauuuaguauauaagguagauauuagaaauuaaauaauauagaaaugaugaauaaccuaucagcuugguaauugccuaaaugcgucuguuuucugugucaauuuuagcguaagcuggccuugacacuuuuaaccggugcaugaauagauuauuuuuaaaauuuguauuuuuacguaaguuugccuacaaaauuuuacauuucgaaacaaucaugaauagaauaacaguaaaagugcguuagucacguggcgcaccuauauuaacuuuuuuaacguuuauuucauuaguauagguuauucauuauaauauauuguacauauacuauaauucccaaauuuauguuuguuauuguacacccccgauuagucgguuuauauuuauauauuuggcucugcgaaugcaguuaaacgcuaccacuuuucggggacuaucuaaucuuuauauaaaauuaguuagucggauuuuagggccuucagcccucuaaucuuuuuauugaaaaaaaaaaaaaaaaaaaa

SEQ ID NO: 99 - RNA, 4105 nt, synthetic construct
  misc_feature (719)..(2910): WCR MV-RNA sequence group ligated into AvrII/KpnI sites of DVVv3-min.
ccauuacggccgggggaaaauaagggcuauggcgucaugccauaguuuauauacaccacggauuacgugaaccuuguuaguaauaacgggugcccuuuguuagggauguguuuuaugaauuauuaauuauguuauaacucuuauuuucaacuacgggauuaaaaccccacuuuaaaacuauauagggaaauuuaguuuaaaccuguauaggaaauuaaggauugauaauuuuauugaacgagccuacucguugaauauuuauaguuaguugugauaacuagccuuguauuuagacauggauaaucuccagcccauguucgacauaccuuaggaaguuuauaugcggcacguauucaauuaaaugaagaugcuauaagugcuugauuagacauuaaaacuagaacaaguucucuaguugucgcugguagagcauaacuuauaguagguggauuuuauuugaugacccaaguauuuaugaaauuugucggccuccaaguugaucagcuaauuauuuauauuuugcgaguaauaguaguucagacugugcuauccccuugacaugcauuuuuugguuacuccagauugugauauuagggcucuaauauugcaagacugcccaccaaguaaggaacuuauguaaacuugcuugggaaacacauuacucaaaaaauuauucuaaggacacuuccucuuuuguaauccguaaagaucgaacaaccuagggcaacgccggucuuauuauacgccaaacaagcuuugccgaucugagccugggagcucucggcaaagcagcuugagaucgaaggagaggagaggaagaggagaguaucucaagcauguuuggcguauuauaccagcacguucguagaagggaucugagccugggagcucucccuucuacgucuaacaugcccucguguagcucauuagcuccgagggcauguuagauguguugguauuauaaauuucucucaaagcuucgggaucugagccugggagcucucccgaagcuuaauugacugcucguguagcucauuagcuccgagcagucaauucagggaaauuuauuaucuuacguugggcaaguuucgaucugagccugggagcucucgaaauuugcgccgaagcuucucguguagcucauuagcuccgagaagcuucggcccaacgugagauuaucuuguccaaaccccagaacgaucugagccugggagcucucguucuggggcguaagcacucucguguagcucauuagcuccgagagugcuuacguugggcaagauuaucggccacacggauccugcgcucguguagcucauuagcuccgagcgcaggauaauccgagauaccgagggaguggugaggaugaggcgaguaucucggaugcguguggccgauuauuuugcaaaauuucuugugcagaucugagccugggagcucucugcauaagagguucccaacucguguagcucauuagcuccgaguugggaaccuaguuugcaaaauuauacuugaagggauacaucaucgaucugagccugggagcucucgaugauguaccccuuggucucguguagcucauuagcuccgagaccaaggggcucuucacguauuauuguguauucaaugguggaaagaucugagccugggagcucucuuuccaccaacgccaagucucguguagcucauuagcuccgagacuuggcguuaaaguacacaauuauccuaguuugcaaaauuucuugaucugagccugggagcucucaagaaauuuauaucaagacucguguagcucauuagcuccgagucuugauauuuuaaacuacgauuauacgccagccacuaguugcggaucugagccugggagcucuccgcaacuaauagugagcucgaaggagaggagaggaagaggagagagcucacuaauggcuggcguauuauugcugaaguucuuugcuucagaucugagccugggagcucucugaagcaaaggcugaucaguuucucguguagcucauuagcuccgagaaauugaucaccaacuucagcaauuauuggaaucuaauuauauuuaugaucugagccugggagcucucauaaauauaauaugaagagaacucguguagcucauuagcuccgaguuuucuucguuuagauuccaauuauaagccaaaauggauucaauugaucugagccugggagcucucgauugaauccgguuaauagcuucucguguagcucauuagcuccgagaaguuauugcauuuuggcuuauuauguuauauacuaaaugcaauagaucugagccugggagcucucuauugcauuuuggcuuugagcacucguguagcucauuagcuccgagugcuuaaaguaaaguauauaacauuauauuaucggccacgcucuagaugcgcucguguagcucauuagcuccgagcgcaucuauuagagagagaccgagggaguggugaggaugaggcgagucucucucuaaagcguggccgauuauuccaugauaucgugaacaugaucugagccugggagcucucauguucacgucauggaugcucguguagcucauuagcuccgagcauccaugauaucgugaaauuauaggggcuaucucuuccuuugaucugagccugggagcucucaaaggaagacacaaauaccucguguagcucauuagcuccgagguauuugugcuagcuccuauuauaccaugguaaauucuucuaugaucugagccugggagcucucauagaagaacaaagaaaacucguguagcucauuagcuccgaguuuucuuugcuaccaugguauuauuguaguugggucuuuuccaggaucugagccugggagcucuccuggaaaagacuaaaccaacucguguagcucauuagcuccgaguugguuuaguagcaacugcaauuaugaccggcguugccuuaggccgguaccccggagcgacggugaaggacggguccaguaguucgcuacuguugaguagagugugagcuccgucgcuccgguuauuggcacguaggagacggucggguccagauauucguaucugucgaguagagugugggcuccuacgugcuuauuggacaugcggagacggucggguccagauauucguaucugucgaguagagugugggcuccgcauguccuuauugggugcauggagacggucggguccagauauucguaucugucgaguagagugugggcuccaugcacccuuauugacgccaacugaaugaaauggugaaggacggguccagguguggcugcuucggcagugcagcuuguugaguagagugugagcuccguaacuagucgcgucuuauugguaaccuauuuggggaccucacuuuuaguugcuagcuaacaaugggggccuuaaaauuaucuuauuaccuuaauugguuguaagugcugaauuuuaagggggccgcgcauauugcguaguaagaccugggccuugcauguagcauuauaagucccacucguuauagcuuaaaucguaccuuagcccguacgagacaagaagugaauuauauuuaaccaccauauauuaaucauuuaguauauaagguagauauuagaaauuaaauaauauagaaaugaugaauaaccuaucagcuugguaauugccuaaaugcgucuguuuucugugucaauuuuagcguaagcuggccuugacacuuuuaaccggugcaugaauagauuauuuuuaaaauuuguauuuuuacguaaguuugccuacaaaauuuuacauuucgaaacaaucaugaauagaauaacaguaaaagugcguuagucacguggcgcaccuauauuaacuuuuuuaacguuuauuucauuaguauagguuauucauuauaauauauuguacauauacuauaauucccaaauuuauguuuguuauuguacacccccgauuagucgguuuauauuuauauauuuggcucugcgaaugcaguuaaacgcuaccacuuuucggggacuaucuaaucuuuauauaaaauuaguuagucggauuuuagggccuucagcccucuaaucuuuuuauugaaaaaaaaaaaaaaaaaaaa

SEQ ID NO: 100 - RNA, 12622 nt, synthetic construct
  5′UTR (1)..(675)
  CDS (676)..(9192): ORF1 polyprotein
  misc_feature (7453)..(9066): RdRp
  misc_feature (7453)..(9066)
  misc_feature (9193)..(11480): Subvrial RNA. WCR MV-RNA group in place of subviral ORF2 structural protein.
  promoter (9193)..(9234): Intergenic region IGR
  3′UTR (11877)..(12622)
ccauuacggccgggggaaaauaagggcuauggcgucaugccauaguuuauauacaccacggauuacgugaaccuuguuaguaauaacgggugcccuuuguuagggauguguuuuaugaauuauuaauuauguuauaacucuuauuuucaacuacgggauuaaaaccccacuuuaaaacuauauagggaaauuuaguuuaaaccuguauaggaaauuaaggauugauaauuuuauugaacgagccuacucguugaauauuuauaguuaguugugauaacuagccuuguauuuagacauggauaaucuccagcccauguucgacauaccuuaggaaguuuauaugcggcacguauucaauuaaaugaagaugcuauaagugcuugauuagacauuaaaacuagaacaaguucucuaguugucgcugguagagcauaacuuauaguagguggauuuuauuugaugacccaaguauuuaugaaauuugucggccuccaaguugaucagcuaauuauuuauauuuugcgaguaauaguaguucagacugugcuauccccuugacaugcauuuuuugguuacuccagauugugauauuagggcucuaauauugcaagacugcccaccaaguaaggaacuuauguaaacuugcuugggaaacacauuacucaaaaaauuauuauggaaaguucaaauaagaauauguguacuggauucccccaaacccuuaaaaucucaacggaaauuaguaucauaaaccaaucaucucaaacccccagagauuggucuuauguacaagaaaauaugcaacaguuuaaaaauuauuuacaauuagcauggauugaaguuccgaauaacauuacaauaggaaauuuugacguuuuugaagauaauaugccaauugcucacgcuauuucaucggauuugcaucaaacagcagguauugcugguguuuuaagacaaaauuauuuuaaaaaucuaccuaaaguuaacguaaauaaaaguaauauuggaaccuuauauuuguauccaaauaaaaauuuaacucgacaaauuauuggauugauuacuaaagauauuuauuauuuaaaaccaacuuaugcuucacuuuuuacaacuuuaauugaauugcguaaauaucuuauuaauaauaaaauaaaaaauauuugccuaccuaugguagguuguaauaaagauaaauuaaauccucuugcaguuuuacccaugcuaauguauugccucggcaauauugaaacucaaguaaaaauaacuaauaaaggaguuuauuuuaauuaucaaccaauuccuguugcuccaaaaauuaauaaaauuaaauuuaguaaacaaauggaaaaaguuaauuaugaucaaacuguagauacuuuugaaacaaauuauguuaaaccuaagaaauucaaagcuaaaguaacauuugaacccucucuuuuaaaucuugauucuaauaguauuauaauuaaugaaaguaagacaguaaaacaaauuuauaaagcuaaauuagaaaaaguaauauaucaaagaucuacaguuuaugaaccacaaaagaagacauaugugguagaagcuuuuucuuguaaacaaccuaaaccaauugucaaaauuuuuaaaaugaaacaagcaauuuuaacucaaacugacuuaaaagaaaauaauaaugaacuaauuaaagaaagaaauaguauugauuauuauaaauuuguuacaccuaacaccguuaaagcuauuaucggauacgacaaaguagaaucaguuuugacauuugauuuggaucguaaacguagauguggagaaaaagcuuuuguacauggaaaaucuuuaaaaucaauagcuaaacgacaacuguguuauaguuauguaccaucucuaaacucagugcaaguauauauuaaaauguuacaauuaacggcgcagaaacaaguauuuggucauguaguaacgaaaauucaagaaaguauugccaauauggauguucauggaaugcaaauuaacaauauaccugaagaauuaaugaaaucauuauccagaacaauuuuagaacaauuacaauuagacacuaaaguaauaaaaccaguuuuguuagcaaauauauuaaauuguuucguaaauguuguuugugccuuaauaauaauauucacugccaaaaguaaaguugcaucuggagcagcuauuguucaaauaauuucaaauauuuuaaccuuaauguuggauauaggcauuuauuauucacuuaaacauaugaccgaagaaagaauuaaagcaauaacuacgacuauaacacaggaugcuguccaaaaaguaaugaaacaacauggcguaacuaugauggcaucugauaaaguaauaacagccaugcaaaaugucgauuuaaaugaugcuaaaucaauaaagcauuuuauaaaagaauucgaaaaagaagaagauaccgacauuucagaaauuucacccgauuguguaccaaacuauucugauauguuaccuauuggauuacaaaugaaaaagaagaacaggcuugauauggauuuuguuuugauggacaaaauagcaguuguugauccaacggguaaguuaaugaagauaguuagugacuguccauacgacacaccuaguaaaaauaaucaguguuugcuccacucccuucugggugcacuagaucauagagauagaaaaguuuuaguggaugaaaguaauauugaaaaauucuuccaauggauguccgauaauauuagugauccagaugugauugaagcaauuaauguuuauaaaauggaguaugcuucugauuucccaccagaagcaacuaauuuagauguuuuacaaggaauuaaaacucauaagaaaauguuaccaguugaccuuuguaaauuacucucaaaauauuuggaccguacuauaauuguaaaacauguaaauaaaguuuugacuuauggaccggaaagaugggauaacaauauaccaaaaauacuccaauuagaaaaucaacauuuuagcuauauugaccgaguacuaguggauaauauuuugccauuaaaagcagcaguugaguguaugguucaauuuacuuuaacacgagccgcacaauuuaauacacccgauacacuuaaacaaaaagaguauuauuuagcgauggcagcagcauacguuuuuacauauaauucuagaauugcuguguguuguaugaaugugaaaucuaauaagcuugaugcgaaaucaauuauugguauuugcacagauauucugucuacaauugcuuucguaguugguaugggaguaucuaguggugauuuugcaucacgcaugguaaaggcugagaaauuuucaacaucuauggauacauuugcaucuaggacaacuaauaaguuagauucgauuuuaucgaaagcuuucgguauuuauaugucggaagauggcgaaacgguugcaaguuuauccaaagauauugauauacuagagaaauauaaagcacauacuguagccgauuauaugaauaauaugucaaaauaucaagaaguuaaaacauauguagaaggaguuacaaaacguaugacaucacuacaccuauuaaauagagauaccacuaaugcuauucaaaauacgcaaaauauuauaagaaagaguugugaugccauaauggcaguuuugaaugacguugauaaguugaauagaauggaaucauugagacaggaauuugugggccuuaagggagcugguaaaaccucaacauugaacaauuaucuaauaccaaauauugcuaagcgcuuggcguugaauccaagcaccuaugcagcagaaauugacgcgaauggacauuggaaacccuauagauaugaagauuuugcuuuaauagaugauuuguuuaaucaggguaguaacgaccccuuuauaccacauauuguaaggauggcuuccucucuuaacuuugauuuaccuggagcugacuugagcaauaaagagaaaacuuuaagggcuagauucuuguuugcgacagcaaacguugcauauguaagaaugaaggaagaaaugcccgcuagugcucaggaaggauuuuaugguagauuuaaguguauucaauuuauuaaugccaaacaagguauuaaucaggcaagagauucaguggaauacaucgacuaugaagaucaugauacauaugaauugagaauguaugaagaacaaauuaaugaacaauucucacagauugaaagaaaugaccauauaacuggugaacguauacaacauuuugauccuucuacuaagacuuaugaugaccauauuuacaaaauaggacauuuacauuauaaaauuguaaccuuuaaacaguuagaaacaauuuguauagaucaguuuaauaaaaaguaucaacauuacaaaauggcuagagaucguaaauguguaguggacuuugaauauuuuagaaauauugauaugaaaucagacgcaauaguagaaaauaugguacguaaggguuaucucccagcagaaauugaagcagcaagacaaacuguaggacuuacagcuggaccagaugaagugcaugccaaaucugaaucuauaauuaaucauuacguuguaucaguuuuuggcgauacuggaacuggaaaaucuguuuuugcuacacagcuggcguuagaucugucagacuuucugaauuugaaaguauauucuguggaagauauaaaugauacggcccauuuagacuacuuaaauccaaguauaauuguuuuccaugauagcauuuaugaugaauugaaauauguugcauuauaugauaaauuaccaaaaccauguuuaauaauaacuacaaguaaucauacauuaaaauggcauagagagaaauuauguaauauuguugauaauaguacuucagcagcagagcgcucuucuagugcuuuguuggaacaugguuucuuuaacaaaaugaaaaauaaaauuggcggacuuguuucuuauuuuaauuugucaaguauuagagaagguuauuugguucaaacauucccaggauaccaaccaacaccugguuauuguagaagacuagguuugaacacugcauuccacccgauaucgacagaagauuugacaucuaauuuaguauauucacagcguccuaauaaagaaacuacaaccauuuuugcaacuucaggauaccauuuccaucaggauaauaaagaagugcaauuaaacgaagucguuaaagaaguuuuaaugaaauauagaggagcuaagaaauucauucagccugauauuaucaaauuaggaguaucagaauuugaggaacaauuuaagaaauuaacaacuauugauguagaaauucagucuaaaaauauugagucauugauuaaauuaacgaacgauagagcuauacuaguaaacucuguaacugcagcuagauuuguuggugguucuaauauuguuaaaguuuccaguagagucagaucaaauaauuauucuuuuaauccucuagauuuugcuauuccuauuaaugcuacuguaucugaugcacuagauauagcaaugaaaucuuauaggcuauuauugagugcaaauguagauuauacaguauuuaucaaaugugaagaauuuucuuguauagguauuaacggcaauauuuacuauacuucaguaccuuuuaauguaacuucuuauucacauauugcaacuaaaguaauaacauuuuauaaaguagauacuaauucagauggaauaacaaauaauucagaauuaaucaaauuuacaccggaugaauaugcuaaauuaauuauuacaggagaaggagaacaaaaacuuuuugauaaguugccuuaugcagcuuguaaauauauuuauggcgcuagagcagagauugaaccacauuuugcgacaacauuuacuaaauacgaaguuagaacagaaguuuaugauaaucauacuagagagaaaauggcuuucuuuguaauuuauucagaauuuaagaaaacaauuugguauaaaauagcuauagguauuuuggcuauaacugcaucaguuuuaauaauugaauuaauaacacaaauuuggaauauguuuauugguuuaaaagaaguuuuuacaaguaccguucaaaaagauaauguuaaaguaguuuaucaaguuuuaucaguuaauccuaaagagcaaucagcagaaguuaaaguuuaugguuuuaaagaugauuuagauucauucucuaauaaagaguuauccaauuuaauuauagaugauauugauguugcgaaauuagaugauucaauccccgcagauacuguaauuaaagccuuggauaugucgcaaaugacugugcauagugauuggaauaagaaagacuuugcuaaaaacaaaguaccuacauugguccauagaauguuggauaugcgcguucauagcgauaaaucccaggauaauaagaaaauuucuaggaaggcuaaccguguggagagaauuuuagaaaauaugacaguuaaaggugaauucgauuuuauuaaacauaaaauuacaaacucucucggcaagguuguuguuuccgcaagcgguguaguuaagacggucguagguaugaacauuaaaggacauuauguaauuuguccagcacauguuuauccaguugauacagcguuccauuaugcaaauuuuaccccguuuaauacggguaaaaauuacacaauggucccaauggucuguaguagaguagaugauuuggcuuuguuuuacauaacugauaaauuacauccauuagcgaaagauauuacacaucauuuugaugaucaggaacguuuauauacucgaccauucgaguugccuuauaugcauuuacgaacugguaauguaguaucuuggaaagguggagaugcuauuggauugcgaucauugguuacaaaaguucaucaagacauguuauuaacagaaggaaaaccuaugguagcaggucaauuuuggaguacgguuacucgccuuacuaaaggcgauuguggauucccaauaauagauacuaacucucauaaaauauuuucuauuuauuguggagauaauaauggcguuaaugcuauuuauggaaauaugguaacaucaacauugguuaagaaaaugauugacuuugcugaagguguaucugaaguacacgcuuuuacucguuauaguccacccauggaagaguucuuugaugcagaagauacugguguuaugaaguuuuccuauccaucuggagaacaacauauuguuaugccuguugaaacuugugauuauaucucaaaagcauaugaaacugaaaacuauuacccuugcauggauggagaauuuacgaggaauaucgcguacaguagugauugucaaaagaacgcauuuaugaaaccuaaauaucaacgaacaccuuuugcugaagaaauuuuaacucaauauccacaaauuaaagaugauuguagaaaugccgcuacugacccaagguuaguagaagauaaaucaaaauuaguaaauucaguuaaaggagacccaucaauuguuaugacacaacuggcaaagauuaaugaccccauagaugcagcugguggacuuaugggugagaaguuuauggaagaagcagcugaaauguuaacagaagaguauauugacuuguauagcgguuuugaccauagauuuuugacuacagaagaaacgauaaaugguauugcaaguccucaaucggauuuacaaggucauuuagaagcuguaaauagagaugcgucugcaggucauugguguaugcuaaauggauaucauaaaauuucugauuuauuagaagaaggugguaguuacccuucaggucaacuacgguaucauuuuaagaaagagaauaaaguaauugcuaaauuagaaaaacauauaucacaacaaaagaagaugugggcaaaagguauucguuacgaagguuuaacuaaagauaauuuaaaagccgaauuaagaccauuugaaaaagugagaauuggaaaaacgcguguauuugaaaguuuugacuucuuuacaaccuuuaaccauagacaacuauuuggaacgauacaagcugcaaugaagaagcagagagcucggggaacaugucaaauuggauuagcucaauaugauuuuacgcacuuguauaauagauugcugcagaccucuccauuugcuguucacggggacuucgaggccugggauaagcauauggauugguuuucaguacaguuggcuuguaagauguggacgaagguuuuauggaaaucaccacaauuccaacauuggaaugauaaaauaaaugcacacaaauuugaaugugaagaaaauuuauaccugauguuggaaacugcaauguuuucuaauguguaugcuguuacuuuggcagauggacauauaauaguuaaagaaagaucucaauguucaggaguaguagaaacgacaacuuuaaauucuacuguuaaugacauuaaguguguggcuguuauauuguaucugauaaagaaacauaacguggugcuuagaacucuaggugcuucggaagcagaugcaaggaaguacuaugaagauuacuuggaaacauuggagcuuucaaagaagaguagaaaagcuuuauuaacaucgaaacauauuuauucaccgauuauaccagauagaagaacgauuaaacaaaacuuugauaugauauauuauggcgaugauauugguauuguaguaaauuuagaauuucuguuuauuguaaauuuuguuucaauuaaaaaugcuuauaaauacuuguucgguauuaauuuggacucaccucauaaagaugggucauuaguaccuuuugugccuuuaaaugaauuggaauuuguuaguagaacuuuugauuaucauucugauacuagaaccguuuauccuaaauuaaaagaacaaucaauuuggagacuuuuguucugguguacuaauuuacguccagagcagauagaauccaauaugauugcaauggcuaacgaauuguuccuuuaugaagaaaguaaauacagauuguuuaguucaauugucaacgcaauaauaucaccauaucugaaacgccaauauaauuggaauuugauauuaccaaguuauagacuuggaaggguagcauuugaggagaugucgcaugaacugaaggaggugccucacuccagcaccuguuuuacugaauuggagauucauucaauuuuacagaaagcgcaaaauauaggaagcaacugacuaaggacacuuccucuuuuguaauccguaaagaucgaacaaccuagggcaacgccggucuuauuauacgccaaacaagcuuugccgaucugagccugggagcucucggcaaagcagcuugagaucgaaggagaggagaggaagaggagaguaucucaagcauguuuggcguauuauaccagcacguucguagaagggaucugagccugggagcucucccuucuacgucuaacaugcccucguguagcucauuagcuccgagggcauguuagauguguugguauuauaaauuucucucaaagcuucgggaucugagccugggagcucucccgaagcuuaauugacugcucguguagcucauuagcuccgagcagucaauucagggaaauuuauuaucuuacguugggcaaguuucgaucugagccugggagcucucgaaauuugcgccgaagcuucucguguagcucauuagcuccgagaagcuucggcccaacgugagauuaucuuguccaaaccccagaacgaucugagccugggagcucucguucuggggcguaagcacucucguguagcucauuagcuccgagagugcuuacguugggcaagauuaucggccacacggauccugcgcucguguagcucauuagcuccgagcgcaggauaauccgagauaccgagggaguggugaggaugaggcgaguaucucggaugcguguggccgauuauuuugcaaaauuucuugugcagaucugagccugggagcucucugcauaagagguucccaacucguguagcucauuagcuccgaguugggaaccuaguuugcaaaauuauacuugaagggauacaucaucgaucugagccugggagcucucgaugauguaccccuuggucucguguagcucauuagcuccgagaccaaggggcucuucacguauuauuguguauucaaugguggaaagaucugagccugggagcucucuuuccaccaacgccaagucucguguagcucauuagcuccgagacuuggcguuaaaguacacaauuauccuaguuugcaaaauuucuugaucugagccugggagcucucaagaaauuuauaucaagacucguguagcucauuagcuccgagucuugauauuuuaaacuacgauuauacgccagccacuaguugcggaucugagccugggagcucuccgcaacuaauagugagcucgaaggagaggagaggaagaggagagagcucacuaauggcuggcguauuauugcugaaguucuuugcuucagaucugagccugggagcucucugaagcaaaggcugaucaguuucucguguagcucauuagcuccgagaaauugaucaccaacuucagcaauuauuggaaucuaauuauauuuaugaucugagccugggagcucucauaaauauaauaugaagagaacucguguagcucauuagcuccgaguuuucuucguuuagauuccaauuauaagccaaaauggauucaauugaucugagccugggagcucucgauugaauccgguuaauagcuucucguguagcucauuagcuccgagaaguuauugcauuuuggcuuauuauguuauauacuaaaugcaauagaucugagccugggagcucucuauugcauuuuggcuuugagcacucguguagcucauuagcuccgagugcuuaaaguaaaguauauaacauuauauuaucggccacgcucuagaugcgcucguguagcucauuagcuccgagcgcaucuauuagagagagaccgagggaguggugaggaugaggcgagucucucucuaaagcguggccgauuauuccaugauaucgugaacaugaucugagccugggagcucucauguucacgucauggaugcucguguagcucauuagcuccgagcauccaugauaucgugaaauuauaggggcuaucucuuccuuugaucugagccugggagcucucaaaggaagacacaaauaccucguguagcucauuagcuccgagguauuugugcuagcuccuauuauaccaugguaaauucuucuaugaucugagccugggagcucucauagaagaacaaagaaaacucguguagcucauuagcuccgaguuuucuuugcuaccaugguauuauuguaguugggucuuuuccaggaucugagccugggagcucuccuggaaaagacuaaaccaacucguguagcucauuagcuccgaguugguuuaguagcaacugcaauuaugaccggcguugccuuaggccgguaccccggagcgacggugaaggacggguccaguaguucgcuacuguugaguagagugugagcuccgucgcaccggaacaaggcacguaggagacggucggguccagauauucguaucugucgaguagagugugggcuccuacgugcuuauuggacaugcggagacggucggguccagauauucguaucugucgaguagagugugggcuccgcauguccuuauugggugcauggagacggucggguccagauauucguaucugucgaguagagugugggcuccaugcacccuuauugacgccaacugaaugaaauggugaaggacggguccagguguggcugcuucggcagugcagcuuguugaguagagugugagcuccguaacuagucgcgucuuauugguaaccuauuuggggaccucacuuuuaguugcuagcuaacaaugggggccuuaaaauuaucuuauuaccuuaauugguuguaagugcugaauuuuaagggggccgcgcauauugcguaguaagaccugggccuugcauguagcauuauaagucccacucguuauagcuuaaaucguaccuuagcccguacgagacaagaagugaauuauauuuaaccaccauauauuaaucauuuaguauauaagguagauauuagaaauuaaauaauauagaaaugaugaauaaccuaucagcuugguaauugccuaaaugcgucuguuuucugugucaauuuuagcguaagcuggccuugacacuuuuaaccggugcaugaauagauuauuuuuaaaauuuguauuuuuacguaaguuugccuacaaaauuuuacauuucgaaacaaucaugaauagaauaacaguaaaagugcguuagucacguggcgcaccuauauuaacuuuuuuaacguuuauuucauuaguauagguuauucauuauaauauauuguacauauacuauaauucccaaauuuauguuuguuauuguacacccccgauuagucgguuuauauuuauauauuuggcucugcgaaugcaguuaaacgcuaccacuuuucggggacuaucuaaucuuuauauaaaauuaguuagucggauuuuagggccuucagcccucuaaucuuuuuauugaaaaaaaaaaaaaaaaaaaa

SEQ ID NO: 101 - protein, 2838 aa, synthetic construct; identical to SEQ ID NO: 95

SEQ ID NO: 102 - skipped

SEQ ID NO: 103 - RNA, 663 nt, Western Corn Rootworm
augagcuuuuuuggaaaauuguucggggggaaaaaggaagagauagccccuaguccuggggaggcuauucaaaaacucagagagacugaagaaauguuaauaaaaaaacaggauuuuuuagaaaagaagauagaagaauuuaccaugguagcaaagaaaaaugcgucgaaaaauaaaagaguugcacuccaagcccucaaaaagaagaaacgauuggaaaagacccaacuacaaauagauggaacccuuacaacuauugaaaugcagagggaagcccucgaaggagcuagcacaaauacugcuguauuagauucuaugaaaaaugcugcagaugcccuuaagaaagcucauaagaauuugaauguagaugauguucacgauaucauggaugacauagccgaacaacacgacauagccaacgaaaucacaaacgcuauuagcaauccugucggauucaccgacgaucuggaugacgaugaauuagaaaaagaauuagaagagcucgaacaagaaggauuggaagaagaccugcuccaagugccagguccaacucaacugccggcugugccugcugaugcaguugcuacuaaaccaaucaaaccagcagcuaaaaaaguugaagaugaugacgauaugaaagaauuggaagccugggccucguaa

SEQ ID NO: 104 - RNA, 2258 nt, Western Corn Rootworm
ggccuucaagcagaauugaagcuacccgucucguaguacccuuagcuugucuguaccagccuauaaaggaacgucuggaucuuccaccaauacaauaugacccuguuuuauguacuagaaauacuuguagagcaauauuaaacccacugugucagguagauuaucgagcaaaacucuggguaugcaacuuuuguuuccagagaaauccauuuccaccucaauaugcugcuauuucagaacaacaucaaccagcggaauugaugccuauguuuuccaccauugaauacacaauaacuagagcucaauguuuaccaccaauauuuuuguauguuguugacaccugcauggaugaugaagaacuggguucccugaaagacucauugcaaaugucccuuaguuuguugccaccuaaugcguuaauaggacuaauaacauuugggaaaaugguucaaguucaugaacuuggcacugaagguuguaguaagucauauguguucagagguacaaaagaucuuagugcuaaacagguucaagaaaugcugggaauaggcaaaguggcuuuaggucagcaagccccucaacagccagggcagccucuaagaccugggcaaaugcaaccuacuguuguugcaccaggaagcagguuucuacaaccuguauccaaaugcgauaugaaucuaacagaccuaauaggagaacaacagaaagauccuuggccuguucaucaggguaaaagguauuuaagaucuacagguguagcuuuaucgauugccauugguuuguuagaauguacauauuccaauacuggcgcccgaguuaugcuauuuguuggaggaccuugcucacaaggaccuggucagguaguuaaugaugauuuaaaacagccuauuagaucacaucaugauauucagaaagauaaugcaaaauauaugaagaaagguauuaaacauuaugaugcguuagcaaugagagccgcaacuaauggucacucuguugauauuuauucuugugcuuuggaucagacaggucugauggaaaugaagcaaugcuguaauucuacugggggacacaugguaaugggggauucauuuaauucuuccuuguuuaagcaaacuuuccaacguguguuuaccagagaucaaaaaagugaucugaaaauggcauuuaacgguacuuuggaagugaaguguucccgagaauuaaaaguucaaggagguaucgguucguguguaucacuuaacgugaagagccccuugguuuccgacacagaaauaggaauggguaauacugugcaauggaaaauguguacuuuaacgccaaguacuaccaugucuuuauucuuugaggucguaaaucaacauucugcucccauaccucaaggugguagagguuguauacaauuuauuacgcaguaccagcauucaaguggucaaagaaaaaucagaguaacaacaguggcucgaaauugggcugacgcaacugcuaauauacaccauaucagugccggauucgaucaagaagcugcugcuguaauaauggcuaggauggccguuuauagggcagaaucugaugauaguccagauguucuuagauggguugacagaaugcugauuagauugugucaaaaauucggagaauacaauaaggacgaccccaauucauucagacuuggucaaaacuucagucuuuacccacaguucauguaucacuuaagaagaucucaauuucuucaaguauucaauaauucuccggacgagacuucauucuacagacacauguugaugagggaagaucuuacucaaucuuugauaaugauucaaccuauuuuguauaguuauaguuucaaugguccaccagagccuguauuacuagauacuagcuccauucaaccugacagaauauuacuuauggauacuuucuuccaaauauuaauuuuccauggagagacuaucgcccaauggcguaguuuaaaauaucaagacaugccagaauaugaaaacuuuagacagcuacuacaggcuccaguagaugaugcacaagaaauuuugcaaacuagguucccaaugccgagauauauugauaccgaacaaggcggaucccaagccagauuuuuguugucgaaaguaaauccaagucaaacucauaacaacauguauuccuacggaggugauucuggagcuccaguuuugacagaugauguaucccuucaaguauucauggaccaucuaaagaaauuggcaguuucguccacagcauaa

SEQ ID NO: 105 - RNA, 1323 nt, Western Corn Rootworm
auaauaaguucgauuuuuuacgaaaaugacaaguaucgagacuguggggaccauuguccugaaauugcugaaguuggugaucaauuugauaugucucaucuuguaccgaaccggauaucaaggcuacuucuugggaguaggaggaaccuggaaucuaaacgaagaaaaaaaucccgaugcagaaauuguggcuuccggcguauucguaggauuuaugauuuacacauucgucucgcugaucagccuuugcuucgcuaguggagaucacaaaacgacauucacugauauucugaugaauauaguagggauuuuuauguggauagcugcuggagcuacagcucuucauuauuggcuuggguacuuguccgaauacaaauacacgacaauagauucugaacgacaaguugguuuggcguuaggagcgauguguauaauaaauggagcggucuaucuuguagacggaguacuuuccgcaaucuuuauccucaaagccaaaaugcaauaacuuucaucguaauauaaauauauuuauuuagguuauauacuuuacuuuaagcagcucaaguauaccgugacaucccacucauacaucaaugucuauaauuguuucaugacaaaucauuuaauaguauuuuaaagcauucauucguucaacaccuuuuuuacauuuuucaaguauuauaucaaucuuuuuaaagaauuguuuggcuauuaaccggauucaauccauauaguuccguaaaauaauuaugcguuuuaauaauuaauguaauuaaaaauaguuaguauauuuuugaaauaaaacaacugccgcaacaaauuagggagaauacuuuugucuccuguaguuuuaucuguuauccaaaguucgauuaaauuguuucaauuuaauguauuuuuccauauaacgcaccaaauagucuuaaaaaaauagugccuguuacuaccuaauacuuucuacguaugccuuaugguuaguaguggcuccaauaggugcauuuuacauuuuucaugaaaauucaauucacauuauuuuccucccguucucuucauauuauauuuauuuauucuuuaauauaguuuugggcgugaagcccaguaaauaccuauaugaaguauauugauggaacugccaugcuugauggguuuugucugcaauuaaaauaaaaccaaaacaaacauuuguacaguauuguaaaauucacaauauuuagguagauguuuaaaauuuaauauuuuuuaagaguuugcauuuauguuaaucguaguuaaguuuuuauaaaaauuuaguaguugugagaaauaaaauaaguaaaagagaaa

SEQ ID NO: 106 - RNA, 34 nt, Human
gggaucauguggguccuauuacauuuuaaaccca

SEQ ID NO: 107 - RNA, 5063 nt, Aedes aegypti
ggccauuacggccggggaauuuguugcgagcacuuguccgaacgccaaguguaacaucgguuauugaaaggaagugaaaauauuugcauuuuauuuucguugugauaggggguuuaucggagaaggauuguuccuuucgagcuuggaguggaaaugaugcaagaaaauaguuucgauuagaagagagcuguuuaacaugugcuuuaguguucuuaagauagaagugauauauugcuagaaaaaaaaucccgaagucagauuccuacaaaaaaaaugaagaacaacgguaacaacuugcaauacuuuacggaaucgucugacgacgaugacgaagaaacgcugaugaucaaaaaaguucagcaggacaccaaacugugggacucguuccgagauccgccggugcagcagacguccgguucggcagccagccaggauuaucugagcguguucaucaaaugucucaaaguguuuacguacauuuucguguuccugguaacgcuggccgccgcgugccucuccaagauguccuuccugcugaugacgucgaacgugaaggagggagucaaaaauagauacugcgaugugcgacaaccggacaaacaguucgaggccaucauuccgacagagcaacgaaucggguggaugugggccaucauuaucucguucaccauuccggagguuggaacacugauucgagcagcucgcauuugcuucuucaagaacauuccacguccaacguucggccaguuggcggucguugcccucauggagggcuuccacgugacggguuuggcuauguuggcguuccuaaucuuucccaauauggacgcuguuaaggcgguaauguugaccaacugugucuguuuggugccggcuauucucgggcugcuuucaagaucaucagcggagucgaagcugccuuucaaguacaccguggauguguuggccaucagugcccaggugacaggauucguuguuuggccuuugcugagcaauucguuugaacugugguuuauuccggccgcuguuuuccugguaucaugccacuggugggaaaacuaccucucacuuaaaagcuauuuucgaccauuugccacgcuggccaccauccgggagaagcugacggaaacucguuacaguaccuaucuguucguaucaccguucaaaguguugcucuucauggcagccggaguguaucuaucgggccaagcgguuccggauuuuuucgccauguucagcaacggcuggggaaaccacacaaucgugguccgugagauggaggccauccucaaugagaaguuucccgauuugagcucgauaacauccgaccuggaaguguuggaaauauucccggucaagaaugcuguucuuuggauuacggugacacacguucugugcucguacguuuguuacauuuucaguaaauuuucgugcaaaauccacauccaaagcuuuucgauggcguuuccaaucaaucugaccguuccggugacgguuacucuccuguugguguuuugcggucuucgagaagcggacguuugcguuuucaacggaauucuaccggauuaucuauucuuccggaugccuccaauauacuaucuguucgauuaugucgucaacgaauuuuccugguugugguugcuguggcugcugucgcagaccuggauaacuaggcaucuuuggaugccgaaaagcgaucgcaaugcgucaacggagaagcucuucguuacuccaauguacaauagcuugcugguggaucagagcgucgcgaugaaccguagacgagaagaucaagaggauuucgugaaaaagauagacauggucaaagugaaggauacggagaaagcgaacgaaaucgacgccaaggcgaacgagcgaaagaacgacacgaucaagccguacgacaggauuccgcagaucuucaucugcgccaccauguggcacgaaaccaaggaagaguugauggaauucuuaaaaucgauccugcgguuggacgaggaccaaugcgcucgccggauggccaugaagcacauucaggcgaccaaggaggacaucgauccggauuauuacgaucuugagacucacaucuucuucgacgaugccuucgucaacgacaaauccaagugugaaagcgcuaacgauucuccacucaacuccuauguaaagacgcuuaucaacaaccuggaagaagcggccuuggaaguuuacaaaacuaaaaugcguaucuaucccccaaccaagaucguaacgccauacgguggccggcucauauggacccuucccggacgaacgaaaaugauagcccaucucaaggauaagaacaaaauucgucauaagaaacguuggucccagguaauguacauguacuaccucuugggcuacaggaucaugcagcugaacaccacucccgagcgcaaaauggugaucgcccagaacaccuaucuucuggcccuggauggcgacaucgauuuucaaccaaaagccgucucccuucuuguggaucguaugaaaguugauccggauuugggugcugccuguggucguauucauccgguuggaaccgguccuaugguuugguaccagaucuucgaguaugcaaucggucauuggcuucagaaagcuacggagcacgugaucggaugcguacuuuguuccccaggaugcuucucgcuguuccgaggacgagcucucauggagaacuccgucaugaagaaguacaccaccaagucugaccaggcccgccacuacguccaguacgaucagggugaagaccgauggcucuguacguugcuucucaagcagaaauuccgaguagaauacucggcugccucggacgcguauacccaugcuccggagggguucaacgaguucuacaaccaacgucgacguuggguuccaucuaccauugccaacaucuucgaccuucuuggagaugccaagcgaguugucaaaaccaacaauaguauuucgaugcccuauaucgucuaccagagcauguugauguucgguacgauccucggcccuggaacgaucuuccucaugaugguuggugcuuuaguugcuguguuucguaucgauaucuggacaucguuccuuuggaacggagugccccucgcgguauucauggccauuugcuucuggaugaagcagaaguaccaacucauggcggccuuuuucaucucggcuagcuauucgcuggugaugauggcugugcugguugguauuguuauccaggucauggaagacggcauccuugcuccaucaucgguguucuucauggcuguagcguugcagauuguugucacaggucugcuucauccucaggaaauggaagcccuuccggcggggcuagucuacuacaucaccaucccauccauguacaugauguuggugaucuacucgguguucaacaugaacgacguaucguggggaacccgugagaacccuacggaagcuccgaaggcugccccagugcaacagguaccgcagggcaagaugcacaaacugcugggcuauuugcgaucuggggaccaagaggacgaaggaucgauuaacuuuucugugagugggcuguucagcugucuguucuguacucauccgaaguccagcgcugagaaggagcagauggugcaggucgcauccucgcuggccgagaucaacuccaaaaugaaggaucuggaacugaaauuaaccggcaccgucaccguaaugagauccuccgacgacgacgaugacguggaugagaugagcugucucgacaugcaaggcggaucuccgaacggaguguccuccccgauucagaccgugaagaacuccgaacuggaagagccggaaaagcaaaucaacuaucuuccggauuggcuuuacgacguagaucugcgaaauggcgacaccgaaaccaucuccgcuucggaagaacaguucuggaucgaguugaucgaaaaguccucaaacccuggaucugacagagaagcagaaagugagaugcaaucccagcugaaaaaccuucgugaucuggccguauuugcuuucgugauggcgaacgcccuauucgugcuggucaucuuccugcugcaguugaacaaaaaggaccugcacauccaguggugguucaacgcgaagaacaccaucagcuucgacgagagcaccguggagauuaucguucgccgggaguucuuaaagaucgaaccuaucggauugguguucguguucuucuucggguugauccucgucauccaguucguugcgaugcugaugcaccgcuucgggacgaucucgcagaucuuggcguccacgcagcugaauugguacuguucgaagaaggcgaaggacaugucccuggaugcggaguugagggagaaugccguggaaaucgcaaggagauugcagcgaccgaagccacagugggaugaagaagaucuggaagacgaucagaaagcgauugggaggagagacacgauccauagaauccuauaccaacacaagaacaaacaggauuggagcaaucuggaggcgaacuucaagcgcaacuacuacaaggacggcgaucugaacuugggaggaagauuaacgcucagucgaaagacacugaauguccuggacacgagaagaaagucgauggccgaacaaaggaaaauucggaagucgauucuucgaggacagaaccccuacgacacggcuggagauuugugguaccaggguagcccagacaguggacucaaucaacggaaaacuucuucuccuccagguuacaacggagcggcaagggguccgccuuccaaucaaucgccaaacuucgaaggaaacaaucgcgguagggugaacuacgcuuaucaaaucgacgaagaacuagaggacuacucggaggaagaagacacgauggaguucgaacguccgacgcugcaaauggagaugaccgaacgaccgacgcccaaauccaaaaggaagagucgaguugcuuuugccuaaacugaaacauaucuuguaguuaaagauucauuauucccccaacacaauaaaaaguaacucaacgaaaaaaaaaaaaaaaaaaaaaaaaaaaaaaa